SEQUENCE LISTING (25 sequences)

SEQ ID NO: 1 - DNA, 606 bp, Homo sapiens
atgagccggagggagggaagtctggaagacccccagactgattcctcagtctcacttcttccccacttggaggccaagatccgtcagacacacagccttgcgcacctcctcaccaaatacgctgagcagctgctccaggaatatgtgcagctccagggagaccccttcgggctgcccagcttctcgccgccgcggctgccggtggccggcctgagcgccccggctccgagccacgcggggctgccagtgcacgagcggctgcggctggacgcggcggcgctggccgcgctgcccccgctgctggacgcagtgtgtcgccgccaggccgagctgaacccgcgcgcgccgcgcctgctgcgccgcctggaggacgcggcgcgccaggcccgggccctgggcgccgccgtggaggccttgctggccgcgctgggcgccgccaaccgcgggccccgggccgagccccccgccgccaccgcctcagccgcctccgccaccggggtcttccccgccaaggtgctggggctccgcgtttgcggcctctaccgcgagtggctgagccgcaccgagggcgacctgggccagctgctgcccgggggctcggcctga

SEQ ID NO: 2 - protein, 201 aa, Homo sapiens
MSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 3 - protein, 201 aa, Artificial Sequence
  Recombinant protein - based on SEQ ID NO2
MSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 4 - protein, 201 aa, Artificial Sequence
  Recombinant protein - based on SEQ ID NO2
MSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVSGLYREWLSRTEGDLGQLLPGGSA

SEQ ID NO: 5 - protein, 201 aa, Artificial Sequence
  Recombinant protein - based on SEQ ID NO2
MSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVSGLYREWLSRTEGDLGQLLPGGSA

SEQ ID NO: 6 - protein, 202 aa, Artificial Sequence
  Recombinant protein - based on SEQ ID NO2
MCSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVSGLYREWLSRTEGDLGQLLPGGSA

SEQ ID NO: 7 - protein, 202 aa, Artificial Sequence
  Recombinant protein - based on SEQ ID NO2
MSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVSGLYREWLSRTEGDLGQLLPGGSAC

SEQ ID NO: 8 - protein, 203 aa, Artificial Sequence
  Recombinant protein - based on SEQ ID NO2
MCSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVSGLYREWLSRTEGDLGQLLPGGSAC

SEQ ID NO: 9 - protein, 200 aa, Artificial Sequence
  Recombinant protein - based on SEQ ID NO2
SRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 10 - protein, 200 aa, Artificial Sequence
  Recombinant protein - based on SEQ ID NO2
SRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVSGLYREWLSRTEGDLGQLLPGGSA

SEQ ID NO: 11 - protein, 200 aa, Artificial Sequence
  Recombinant protein - based on SEQ ID NO2
SRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVSGLYREWLSRTEGDLGQLLPGGSA

SEQ ID NO: 12 - protein, 201 aa, Artificial Sequence
  Recombinant protein - based on SEQ ID NO2
SRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSAC

SEQ ID NO: 13 - protein, 201 aa, Artificial Sequence
  Recombinant protein - based on SEQ ID NO2
SRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVCGLYREWLSRTEGDLGQLLPGGSAC

SEQ ID NO: 14 - protein, 201 aa, Artificial Sequence
  Recombinant protein - based on SEQ ID NO2
SRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVSGLYREWLSRTEGDLGQLLPGGSAC

SEQ ID NO: 15 - protein, 201 aa, Artificial Sequence
  Recombinant protein - based on SEQ ID NO2
SRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVSGLYREWLSRTEGDLGQLLPGGSAC

SEQ ID NO: 16 - protein, 201 aa, Artificial Sequence
  Recombinant protein - based on SEQ ID NO2
CSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 17 - protein, 201 aa, Artificial Sequence
  Recombinant protein - based on SEQ ID NO2
CSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVSGLYREWLSRTEGDLGQLLPGGSA

SEQ ID NO: 18 - protein, 201 aa, Artificial Sequence
  Recombinant protein - based on SEQ ID NO2
CSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVSGLYREWLSRTEGDLGQLLPGGSA

SEQ ID NO: 19 - protein, 202 aa, Artificial Sequence
  Recombinant protein - based on SEQ ID NO2
CSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSAC

SEQ ID NO: 20 - protein, 202 aa, Artificial Sequence
  Recombinant protein - based on SEQ ID NO2
CSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVCGLYREWLSRTEGDLGQLLPGGSAC

SEQ ID NO: 21 - protein, 202 aa, Artificial Sequence
  Recombinant protein - based on SEQ ID NO2
CSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVSGLYREWLSRTEGDLGQLLPGGSAC

SEQ ID NO: 22 - protein, 202 aa, Artificial Sequence
  Recombinant protein - based on SEQ ID NO2
CSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVSRRQAELNPRAPRLLRRLEDAARQARALGAAVEALLAALGAANRGPRAEPPAATASAASATGVFPAKVLGLRVSGLYREWLSRTEGDLGQLLPGGSAC

SEQ ID NO: 23 - protein, 203 aa, Artificial Sequence
  Recombinant protein - based on SEQ ID NO2
XXSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSAX

SEQ ID NO: 24 - protein, 200 aa, Artificial Sequence
  Recombinant protein
MSRREGSLDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 25 - protein, 199 aa, Artificial Sequence
  Recombinant protein
SRREGSLDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSA

(19) United States
(12) Patent Application Publication
(10) Pub. No.: US 2014/0171625 A1
(43) Pub. Date: Jun. 19, 2014

(54) CARDIOTROPHIN RELATED MOLECULES FOR ENHANCED THERAPEUTICS

(71) Applicants: Tom Tong Lee, San Diego, CA (US); Kevin Lai, San Diego, CA (US); John Mendlein, Encinitas, CA (US); Peter Flynn, La Jolla, CA (US)

(72) Inventors: Tom Tong Lee, San Diego, CA (US); Kevin Lai, San Diego, CA (US); John Mendlein, Encinitas, CA (US); Peter Flynn, La Jolla, CA (US)

(73) Assignee: Fate Therapeutics, Inc., San Diego, CA (US)

(21) Appl. No.: 13/983,282

(22) PCT Filed: Feb. 1, 2012
(86) PCT No.: PCT/US12/23534

§ 371 Date: Feb. 7, 2014

Related U.S. Application Data
(60) Provisional application No. 61/438,581, filed on Feb. 1, 2011.
(60) Provisional application No. 61/438,607, filed on Feb. 1, 2011.
(60) Provisional application No. 61/439,803, filed on Feb. 4, 2011.
(60) Provisional application No. 61/439,801, filed on Feb. 4, 2011.

(51) Int. Cl. C07K 14/52

(52) U.S. Cl. CPC C07K 14/52
USPC 530/399; 536/23.5; 435/320.1; 435/254.21; 435/348; 435/419; 435/325; 435/365.1; 435/369; 435/352; 435/364; 435/367; 435/254.23; 435/360; 435/252.33

(57) ABSTRACT

The invention provides novel polypeptides having at least one biological activity of cardiotrophin and improved biologic drug-like properties, and polynucleotides encoding the polypeptides of the invention. The polypeptides of the invention can be used therapeutically, such as, for example, in methods of tissue regeneration.

### CROSS REFERENCE TO RELATED APPLICATIONS

This application claims the benefit under 35 U.S.C. §119(e) of U.S. Provisional Application No. 61/438,581, filed Feb. 1, 2011, Provisional Application No. 61/438,607, filed Feb. 1, 2011, Provisional Application No. 61/439,801, filed Feb. 4, 2011, and Provisional Application No. 61/439,803, filed Feb. 4, 2011, each of which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is FATE_{—}096_{—}02WO_ST25.txt. The text file is 46 KB, was created on Feb. 1, 2012, and is being submitted electronically via EFS-Web.

### BACKGROUND

1. Technical Field

The invention relates generally to novel polypeptides having at least one biological activity of cardiotrophin and having desirable pharmacological properties. The polypeptides of the invention and compositions thereof may be used therapeutically, for example for promoting tissue protection and/or regeneration.

2. Description of the Related Art

Cardiotrophin-1 (CT-1), also called cardiac hypertrophy factor, has yet to find therapeutic application. CT-1 polypeptide is a small secreted protein of 21.2 KDa originally identified as an activator of cardiomyocyte hypertrophy in vitro. The genes encoding both human and mouse CT-1 have been cloned (Pennica, D., et al., (1996) Cytokine, 8:183-189; Pennica, D., et al., (1995) Proc. Natl. Acad. Sci. USA, 92:1142-1146).

CT-1 is a member of a family of cytokines that includes interleukin-6 (IL6), leukemia inhibitory factor (LIF), ciliary neurotrophic factor (CNTF), oncostatin-M (OSM), cardiotrophin-like cytokine (CLC), neuropoietin (NP) and interleukin-11 (IL11). The members of this cytokine family signal via a common gp130 receptor either as gp130 homodimers or via more cytokine specific receptor heterodimer complexes such as LIF receptor/gp130. The receptors of the IL-6 family of cytokines are composed of distinct subunits, but all receptors in the class share the gp130 subunit (Kishimoto, T., et al., Cell 1994 Jan. 28; 76(2):253-262). Some members of the family (IL-6 and IL-11) induce homodimerization of gp130 (Murakami, M., et al., Science. 1993 Jun. 18; 260(5115):1808-1810), whereas others such as leukemia inhibitory factor (LIF), oncostatin and ciliary neurotrophic factor (CNTF) induce heterodimerization of the gp130 subunit with the 190 kDa LIF receptor (Murakami 1993). The CT-1 receptor contains the gp130 chain, the β subunit of the LIF receptor (LIFRβ) and possibly a third component known as the α subunit of the CT-1 receptor (Pennica, D, et al., Proc Natl Acad Sci USA. 1995 Feb. 14; 92(4):1142-1146; Robledo, 0., et al., J. Biol. Chem. 1997, 272(8): 4855-4863). The α subunit may participate in the formation of a three-part complex that confers high sensitivity and specificity to CT-1.

CT-1 signaling results in the activation of key intracellular molecules and pathways such as JAK/Stat3, PI3-Kinase/AKT and ERK. CT-1 signaling mechanisms and ischemic protection have been described in cardiac, hepatic and neural tissues (Wollert et al. 1996; Sheng et al. 1997). U.S. Pat. Nos. 5,534,615; 5,571,675; 5,571,893; 5,624,806 and 5,679,545 describe the use of CT-1 and its antagonists in heart failure, arrhythmic or inotropic disorders, or peripheral neuropathies. The use of CT-1 in the diagnosis and treatment of cancer has also been described.

CT-1 has been described as a potent anti-apoptotic cytokine, protecting cells in vitro and tissues in vivo from ischemic damage (see, e.g., Liao, Z., et al., (2002) Cardiovasc. Res., 53:902-910; Jin, H., et al., Cytokine 1998; 10: 19-25; Latchman, D S., et al., Int. J. Exp. Pathol. 1999; 80: 189-196; Jougsaki M, et al., Circulation 2000; 101: 14-17; Hishinuma S, et al., Biochem. Biophysic. Res. Commun 1999; 264: 436-440; Stephanou, A., et al., J. Mol. Cell. Cardiol. 1998; 30: 849-855). It has also been reported that CT-1 provides protection of hepatocytes against a variety of hepatotoxic agents, reducing hepatocellular apoptosis/necrosis resulting from agents that cause cell death, and that CT-1 may stimulate hepatic regeneration (U.S. Pat. No. 7,732,397). CT-1 therefore represents a protein of interest for therapeutic development.

Although CT-1 was discovered in the mid-nineties, therapeutic forms of this molecule are lacking in the literature and field. Advancement of CT-1 as a potential therapeutic is hampered by lack of a reliable isolated wild type protein for in vivo and cell based work, as well human therapeutic assessment. Advancement of CT-1 has also been hampered by the potential for multiple in vivo activities and potential harmful effects, such as inflammatory responses. Assessment of harmful versus beneficial effects of CT-1 related proteins are potentially challenged by:

1) aggregation levels of the isolated protein that can lead to immune system related responses;

2) endotoxin levels that may induce production of inflammatory or pro-inflammatory cytokines, such as IL-6, TNF and LIF;

3) instability of CT-1 protein that is improperly folded or not produced at 37° C. at physiologically relevant pH; and

4) formulation with carrier proteins which may induce production of inflammatory or pro-inflammatory cytokines.

The art has yet to provide enhanced CT-1 related molecules to permit development of CT-1 as a therapeutic. Improved CT-1 related compositions may provide, for example:

1) different half lives in plasma to achieve different exposure profiles over time to provide different therapeutic benefits;

2) the ability to avoid potential deleterious effects reported with high levels of CT-1 wild type administered in vivo, such as cardiac hypertrophy;

3) the ability to modulate undesired cytokine levels that may be associated with CT-1; and

4) the ability to controllably conjugate CT-1 related molecules, such as by site specific pegylation, while retaining desired activities.

Further, formulations and compositions of CT-1 polypeptides that decrease clearance and/or absorption rates and therefore increase systemic half-life and administration interval would be advantageous because CT-1 has a low molecular weight (21.2 KDa). The low molecule weight of CT-1 may result in rapid clearance from the body leading to a short systemic half-life and bioavailability on in vivo delivery.

Several methods may prolong the plasma half-life of certain intravenously administered polypeptides, although sometimes at the expense of desired properties. These include modification of the polypeptide with polyethylene glycol (PEG), dextran, poly[Glu-Lys], pullulan, modified polyaspartate or fatty acids, as well as coupling with gamma-globulin. Modification of polypeptides, however, can result in a significant reduction in their bioactivity, distribution, and/or stability. This is especially the case for small polypeptides where the addition of a large modification, such as a PEG of size relevant to alter pharmacokinetic properties, could cause steric hindrance, blocking or reducing a bioactive polypeptides enzymatic or signaling function.

For example, a number of problems have been observed with PEGylation. Acylation of tyrosine residues on the protein can result in a lowering of the biological activity of the protein; certain PEG-protein conjugates are insufficiently stable and therefore find no pharmacological use; certain reagents used for PEGylation are insufficiently reactive and therefore require long reaction times during which protein denaturation and/or inactivation can occur. Also, the PEGylating agent may be insufficiently selective. Difficulties can also arise as a result of the hydrophobicity of the protein to be PEGylated; in an aqueous medium hydrophobic proteins resist PEGylation at physiological pH. For example, WO87/00056 describes an adverse effect of PEGylation on the in vitro activity of a PEGylated protein, demonstrating that PEGylation decreased the activity of the protein by nearly 10-fold. Additionally, the covalent modification of lysine residues is known to be associated with a reduction in bioactivity of certain proteins. Lysine modification with activated PEG-esters is random, difficult to control, and often results in reduced bioactivity of the modified protein. Goodson, R., et al., Bio/Technology, 8:343 (April 1990). No evidence is presented in the publication that PEGylated derivatives were actually obtained; nor is there any evidence in the publication that these proposed modified polypeptides retained any biological activity.

Given the broad range of differences in the physical characteristics and pharmacokinetics among proteins, it is impossible to predict in advance whether a protein, particularly a protein having low molecule weight such as CT-1, can be successfully modified, such as by PEGylation, and/or whether the modified protein will still retain its biological activity.

CT-1 thus has use as a potential therapeutic in a variety of disorders, and novel CT-1 variants with improved pharmacokinetic properties, such as increased plasma half-life, and/or enhanced activity are highly desirable.

### BRIEF SUMMARY

The present invention generally relates to improved polypeptides and proteins having at least one biological activity of CT-1, and compositions comprising the same. In various embodiments, the present invention contemplates, in part, a composition, comprising an isolated protein of at least 160 identical amino acids of the amino acid sequence set forth in SEQ ID NO: 2 and a protein modification of SEQ ID NO: 2 that includes one or more of the following: a Cys or a Lys at a non-naturally occurring amino acid position in SEQ ID NO: 2; at least one deletion of Cys 105 or Cys 178; at least one substitution of Cys 105 or Cys 178 with another amino acid; and addition of a pharmokinetic (PK) modulator.

In a particular embodiment, a composition comprises an isolated protein at least 160 contiguous amino acids identical to the amino acid sequence set forth in SEQ ID NO: 2.

In a particular embodiment, a composition comprises an isolated protein comprising 161, 162, 163, 164, 165, 166, 167, 168, 169, 170, 171, 172, 173, 174, 175, 176, 177, 178, 179, 180, 181, 182, 183, 184, 185, 286, 187, 188, 189, 190, 191, 192, 193, 194, 195, 196, 197, 198, 199, 200, or 201 contiguous amino acids identical to the amino acid sequence set forth in SEQ ID NO: 2.

In another particular embodiment, a composition comprises an isolated protein comprising 161, 162, 163, 164, 165, 166, 167, 168, 169, 170, 171, 172, 173, 174, 175, 176, 177, 178, 179, 180, 181, 182, 183, 184, 185, 286, 187, 188, 189, 190, 191, 192, 193, 194, 195, 196, 197, 198, 199, 200, or 201 amino acids identical to the amino acid sequence set forth in SEQ ID NO: 2 and further comprises at least one disruption of 1 to 7 amino acids in the contiguous amino acid sequence set forth in SEQ ID NO: 2.

In an addition embodiment, a composition comprises an isolated protein comprising at least 160 contiguous amino acids identical to the amino acid sequence set forth in SEQ ID NO: 2; and at least one disruption of 1 to 7 amino acids in the contiguous amino acid sequence set forth in SEQ ID NO: 2.

In a certain embodiment, a Cys residue at a non-naturally occurring amino acid position in SEQ ID NO: 2 is within about 10 amino acids of the N- or C-terminus of the isolated protein.

In an additional embodiment, the isolated protein comprises a PK modulator that is a polyethylene glycol (PEG) and an isolated protein that comprises Cys 105 and Cys 178 substituted with glycine (G), serine (S), alanine (A), threonine (T), leucine (L), isoleucine (I), or arginine (R).

In a further embodiment, the isolated protein comprises a PK modulator that is a PEG and an isolated protein that comprises either Cys 105 and Cys 178 substituted with G, S, A, T, L, I, or R.

In a certain embodiment, the isolated protein comprises a PK modulator comprising a PEG of at least 10 kD, about 1 PEG moiety per about 1 molecule of the isolated protein, and the isolated protein is at least 95% pure by weight.

In a particular embodiment, the isolated protein comprises a PK modulator comprising a PEG of at least 10 kD, about 2 PEG moieties per about 1 molecule of the isolated protein, and the isolated protein is at least 95% pure by weight.

In another particular embodiment, the isolated protein comprises a PK modulator comprising a PEG of at least 10 kD, about 0.5 PEG moieties per about 1 molecule of the isolated protein, and the isolated protein is at least 95% pure by weight.

In one particular embodiment, the isolated protein comprises a PK modulator that is a PEG and either Cys 105 and Cys 178 are substituted with G, S, A, T, L, I, or R and the isolated protein has activity in a cell-based or biochemical assay.

In various embodiments, the present invention contemplates, in part, a composition, comprising an isolated protein that includes 80% identity to the amino acid sequence set forth in SEQ ID NO: 2, is at least about 95% pure by weight and comprises a protein modification of SEQ ID NO: 2 that includes one or more of the following a Cys or a Lys at a non-naturally occurring amino acid position in SEQ ID NO: 2; at least one deletion of Cys 105 or Cys 178; at least one substitution of Cys 105 or Cys 178 with another amino acid; and addition of a pharmokinetic (PK) modulator.

In a particular embodiment, a composition comprises an isolated protein comprises between 170-201 amino acids identical to the amino acid sequence set forth in SEQ ID NO: 2; at least one deletion of an amino acid; and at least one addition of an amino acid.

In a particular embodiment, a composition comprises an isolated protein comprises between 170-199 amino acids identical to the amino acid sequence set forth in SEQ ID NO: 2; at least one deletion of an amino acid; and at least one addition of an amino acid.

In another particular embodiment, a composition comprises an isolated protein comprises between 170-201 amino acids identical to the amino acid sequence set forth in SEQ ID NO: 2; only one Cys residue; and is substantially endotoxin free.

In another particular embodiment, a composition comprises an isolated protein comprises between 170-200 amino acids identical to the amino acid sequence set forth in SEQ ID NO: 2; only one Cys residue; and is substantially endotoxin free.

In one embodiment, an isolated protein comprises a Cys at a non-naturally occurring amino acid position in SEQ ID NO: 2 and is within about 10 amino acids of the N- or C-terminus of the isolated protein, wherein the Cys is at its naturally occurring position or within 5 amino acid positions of its naturally occurring position.

In one embodiment, an isolated protein comprises a Cys at a non-naturally occurring amino acid position in SEQ ID NO: 2 and is within about 10 amino acids of the N- or C-terminus of the isolated protein or within 5 amino acid positions of its naturally occurring position.

In one embodiment, an isolated protein comprises a Cys at its naturally occurring position or within 5 amino acid positions of its naturally occurring position.

In a certain embodiment, the isolated protein comprises a PK modulator that is a PEG and Cys 105 and Cys 178 are substituted with G, S, A, T, L, I, or R.

In an additional particular embodiment, the isolated protein comprises a PK modulator comprising a PEG of at least 10 kD, about 1 PEG moiety per about 1 molecule of the isolated protein, and the isolated protein is at least 95% pure by weight.

In one particular embodiment, a composition comprises an isolated protein that comprises about 5% or less protein aggregates.

In a further embodiment, the isolated protein comprises a PK modulator that extends the half life of the protein in a rat by 3 times compared to the half life of the protein without the PK modulator.

In another embodiment, the isolated protein concentration is at least 1 mg/ml, comprises about 5% or less protein aggregates, is stable for at least 12 hours at 37° C. and is formulated at a pH between about 6.5 and about 8.

In another embodiment, the isolated protein concentration is at least 1 mg/ml, comprises about 5% or less protein aggregates, is stable for at least 10 hours at 37° C. and is formulated at a pH between about 6.5 and about 8.

In another embodiment, the isolated protein concentration is at least 1 mg/ml, comprises about 5% or less protein aggregates, is stable for at least 8 hours at 37° C. and is formulated at a pH between about 6.5 and about 8.

In another embodiment, the isolated protein concentration is at least 1 mg/ml, comprises about 5% or less protein aggregates, is stable for at least 6 hours at 37° C. and is formulated at a pH between about 6.5 and about 8.

In a particular embodiment, the isolated protein comprises a PK modulator that is a PEG of about 40 kD or greater and either Cys 105 and Cys 178 are substituted for G, S, A, T, L, I, or R and said isolated protein has activity in a cell based or biochemical assay.

In various other embodiments, the present invention provides, in part, an isolated biologically active polypeptide comprising an amino acid sequence as set forth in SEQ ID NO: 23, wherein Xaa at position 1 is absent or methionine; Xaa at position 2 is absent or any amino acid; Xaa at position 203 is absent or any amino acid; and wherein the polypeptide comprises one or more amino acid substitutions, deletions, or modifications at amino acid positions 106 or 179.

In one embodiment, Xaa at position 2 is selected from the group consisting of: cysteine, lysine, histidine, asparagine, aspartate, glutamate, alanine, glycine, threonine, serine, and valine.

In another embodiment, Xaa at position 203 is cysteine.

In a further embodiment, the polypeptide comprises a chemical modification.

In a certain embodiment, the chemical modification comprises pegylation.

In a certain particular embodiment, the pegylation is site-specific.

In one embodiment, the polypeptide is pegylated at one or more cysteine residues.

In a particular embodiment, the polypeptide comprises a pegylated cysteine at amino acid position 106.

In an additional embodiment, the polypeptide comprises an amino acid sequence as set forth in any one of SEQ ID NOs: 4, 10, 12, 14, 17, 19, and 21.

In a further embodiment, the polypeptide comprises a pegylated cysteine at amino acid position 179.

In another particular embodiment, the polypeptide comprises an amino acid sequence as set forth in any one of SEQ ID NOs: 3, 9, 12, 13, 16, 19, and 20.

In a certain particular embodiment, the polypeptide comprises a pegylated cysteine at amino acid position 203.

In another certain particular embodiment, the polypeptide comprises an amino acid sequence as set forth in any one of SEQ ID NOs: 7-8, 12-15, and 19-22.

In one embodiment, the polypeptide comprises pegylated cysteines at amino acid positions 106, 179, and 203.

In a further embodiment, the polypeptide comprises an amino acid sequence as set forth in any one of SEQ ID NOs: 3-22.

In a particular embodiment, the polypeptide comprises a chemical modification.

In another particular embodiment, the polypeptide comprises an amino acid sequence as set forth in any one of SEQ ID NOs: 3-4, 6-10, or 12-22, and wherein the polypeptide is pegylated at one or more cysteine residues.

In one embodiment, the invention provides a polypeptide comprising the amino acid sequence of SEQ ID NO: 3 wherein the polypeptide comprises a pegylated cysteine at amino acid position 178.

In one embodiment, the invention provides a polypeptide comprising the amino acid sequence of SEQ ID NO: 4 wherein the polypeptide comprises a pegylated cysteine at amino acid position 105.

In one embodiment, the invention provides a polypeptide comprising the amino acid sequence of SEQ ID NO: 6 wherein the polypeptide comprises a pegylated cysteine at amino acid position 2.

In one embodiment, the invention provides a polypeptide comprising the amino acid sequence of SEQ ID NO: 7 wherein the polypeptide comprises a pegylated cysteine at amino acid position 202.

In one embodiment, the invention provides a polypeptide comprising the amino acid sequence of SEQ ID NO: 8 wherein the polypeptide comprises a pegylated cysteine at one or more of amino acid positions 2 and 203.

In one embodiment, the invention provides a polypeptide comprising the amino acid sequence of SEQ ID NO: 9 wherein the polypeptide comprises a pegylated cysteine at amino acid position 177.

In one embodiment, the invention provides a polypeptide comprising the amino acid sequence of SEQ ID NO: 10 wherein the polypeptide comprises a pegylated cysteine at amino acid position 104.

In one embodiment, the invention provides a polypeptide comprising the amino acid sequence of SEQ ID NO: 12 wherein the polypeptide comprises a pegylated cysteine at one or more of amino acid positions of 104, 177, and 201.

In one embodiment, the invention provides a polypeptide comprising the amino acid sequence of SEQ ID NO: 13 wherein the polypeptide comprises a pegylated cysteine at one or more of amino acid positions 177 and 201.

In one embodiment, the invention provides a polypeptide comprising the amino acid sequence of SEQ ID NO: 14 wherein the polypeptide comprises a pegylated cysteine at one or more of amino acid positions 104 and 201.

In one embodiment, the invention provides a polypeptide comprising the amino acid sequence of SEQ ID NO: 15 wherein the polypeptide comprises a pegylated cysteine at amino acid position 201.

In one embodiment, the invention provides a polypeptide comprising the amino acid sequence of SEQ ID NO: 16 wherein the polypeptide comprises a pegylated cysteine at one or more of amino acid positions 1 and 178.

In one embodiment, the invention provides a polypeptide comprising the amino acid sequence of SEQ ID NO: 17 wherein the polypeptide comprises a pegylated cysteine at one or more of amino acid positions 1 and 105.

In one embodiment, the invention provides a polypeptide comprising the amino acid sequence of SEQ ID NO: 18 wherein the polypeptide comprises a pegylated cysteine at amino acid position 1.

In one embodiment, the invention provides a polypeptide comprising the amino acid sequence of SEQ ID NO: 19 wherein the polypeptide comprises a pegylated cysteine at one or more of amino acid positions 1, 105, 178, and 202.

In one embodiment, the invention provides a polypeptide comprising the amino acid sequence of SEQ ID NO: 20 wherein the polypeptide comprises a pegylated cysteine at one or more of amino acid positions 1, 178, and 202.

In one embodiment, the invention provides a polypeptide comprising the amino acid sequence of SEQ ID NO: 21 wherein the polypeptide comprises a pegylated cysteine at one or more of amino acid positions 1, 105, and 202.

In one embodiment, the invention provides a polypeptide comprising the amino acid sequence of SEQ ID NO: 22 wherein the polypeptide comprises a pegylated cysteine at one or more of amino acid positions 1 and 202. In yet another particular embodiment, the polypeptide has increased biological activity compared to the polypeptide of SEQ ID NO: 2.

In a certain embodiment, the polypeptide has improved stability, solubility, or pharmacokinetic properties compared to the polypeptide of SEQ ID NO: 2. In particular embodiments, the present invention contemplates polynucleotides encoding any of the foregoing polypeptides.

In other particular embodiments, the present invention contemplates vectors comprising the polynucleotides of the invention.

In certain embodiments, the present invention contemplates host cells comprising the vectors of the invention.

In other certain embodiments, the present invention contemplates polypeptides produced by the host cells of the invention.

In one embodiment, the present invention provides an expression system for expressing a polynucleotide encoding a polypeptide of the invention comprising a vector comprising a polynucleotide of the invention.

In a particular embodiment, the expression system comprises a mammalian cell, a bacterial cell, a yeast cell, a plant cell, or an insect cell.

In a certain embodiment, the host cell comprises a vector comprising a polynucleotide of the invention.

In an additional embodiment, the host cell is a mammalian cell or a bacterial cell.

In one embodiment, the present invention provides a polypeptide produced by an expression system or a host cell of the invention.

In various other embodiments, the present invention contemplates, in part, compositions of the invention, compositions comprising the polypeptides of the invention; compositions comprising polynucleotides encoding the polypeptides the invention; compositions comprising a vector comprising a polynucleotide encoding a polypeptide of the invention.

In one embodiment, the composition comprises a pharmaceutically-acceptable salt, carrier, or excipient.

In a particular embodiment, the composition is soluble in an aqueous solution.

In another particular embodiment, the composition is formulated for injection.

In a certain embodiment, the composition is formulated for one or more of intravenous injection, intracardiac injection, subcutaneous injection, intraperitoneal injection, or direct injection into a tissue.

In a certain particular embodiment, the composition promotes tissue growth, regeneration, maintenance or repair.

In various other embodiments, the present invention contemplates, in part, a method of treating, preventing, or alleviating a medical condition comprising administering a composition of the invention

In one embodiment, the medical condition is selected from the group consisting of ischemic cardiac disease, myocardial infarction, artherosclerosis, hypertension, restenosis, angina pectoris, rheumatic heart disease, congenital cardiovascular defects, arterial inflammation, disease of the arteries, arterioles and capillaries, degenerative liver disease, cirrhosis, hepatitis, and diabetes.

In a particular embodiment, the composition is administered to a patient within one week of the occurrence of a medical procedure, occurrence of injury or onset of disease.

In a certain embodiment, the composition is administered to a patient within one week of a myocardial infarction.

In various embodiments, the present invention provides a method of producing a polypeptide comprising culturing a host cell of the invention under conditions suitable for expression of the polypeptide.

In one embodiment, the process for producing a polypeptide comprises one or more of the following steps: i) expression of a polynucleotide encoding a polypeptide according to SEQ ID NO: 2-22, and 24-25 in a host cell; ii) culturing the host cell to express the polypeptide as inclusion bodies; iii) washing the inclusion bodies; iv) solubilizing the polypeptide and v) refolding the polypeptide.

In various other embodiments, the present invention provides a polypeptide produced by the process comprising two or more of the following steps: i) expression of a polynucleotide encoding a polypeptide according to SEQ ID NO: 2-22, and 24-25 in a host cell; ii) culturing the host cell to express the polypeptide as inclusion bodies; iii) washing the inclusion bodies; iv) solubilizing the polypeptide and v) refolding the polypeptide.

In one embodiment, the invention provides a composition comprising an isolated polypeptide comprising the amino acid sequence set forth in any one of SEQ ID NOs: 2-22, and 24-25; wherein the isolated polypeptide further comprises:

a) an endotoxin profile of less than about 5 EU per mg;

b) an aggregation profile of less than about 10% at 22° C.; and

wherein the polypeptide is soluble in a therapeutically acceptable formulation.

In particular embodiments, the endotoxin profile is measured by Limulus Amebocyte Lysate assay (LAL). In some embodiments, the aggregation profile is measured by SEC-HPLC.

In some embodiments of the invention, the isolated polypeptide does not have an N-terminal methionine. In yet other embodiments, the therapeutically acceptable formulation does not comprise a carrier protein.

In some embodiments, the isolated polypeptide is produced in bacteria. In further embodiments, the isolated polypeptide is not glycosylated and is stable at 37° C. for at least 12 hours in the absence of a carrier protein.

In other embodiments, the isolated polypeptide comprises an aggregation profile of less than about 5%. In further embodiments of the invention, the isolated polypeptide is formulated with a pH of between about 6 and 8 and a polypeptide concentration of at least about 1 mg/ml. In other embodiments, the polypeptide concentration is at least about 4 mg/ml.

In yet other embodiments, the isolated polypeptide when injected into a rat induces less secretion of at least one pro-inflammatory cytokine within 2 hours than a comparable polypeptide that has an endotoxin profile of about 10 EU per mg. In some embodiments of the invention, the isolated polypeptide when injected into a rat induces less secretion of at least one pro-inflammatory cytokine within 2 hours than a comparable polypeptide that has an endotoxin profile of about 5 EU per mg. Another embodiment of the invention provides a composition comprising an isolated polypeptide comprising at least 150 contiguous amino acids of the amino acid sequence set forth in SEQ ID NOs: 2-22, and 24-25; wherein the isolated polypeptide further comprises:

a) an endotoxin profile of less than about 5 EU per mg;

b) an aggregation profile of less than about 10% at 22° C.; and

wherein the polypeptide is soluble in a therapeutically acceptable formulation; and

wherein the composition is substantially free of mammalian pro-inflammatory agents.

In one embodiment, the therapeutically acceptable formulation does not comprise a carrier protein. In some embodiments, the isolated polypeptide is produced in bacteria.

In some embodiments, the isolated polypeptide is not glycosylated, is made in inclusion bodies, and is stable at 37° C. for at least 12 hours in the absence of a carrier protein. In other embodiments, the isolated polypeptide comprises an aggregation profile of less than about 5% at 37° C. In yet other embodiments, the isolated polypeptide is formulated with a pH of between about 6 and 8, an ionic strength of at least about 50 mM and a polypeptide concentration of at least about 1 mg/ml.

In further embodiments of the invention, the isolated polypeptide when injected into a rat induces less secretion of at least one pro-inflammatory cytokine within 2 hours than a comparable polypeptide with an endotoxin profile of about 10 EU per mg. In other embodiments, the isolated polypeptide when injected into a rat induces less secretion of at least one pro-inflammatory cytokine within 2 hours than a comparable polypeptide with an endotoxin profile of about 5 EU per mg.

In some embodiments, the isolated protein has an aggregation profile of less than about 4% at 37° C. In yet other embodiments, the proinflammatory agent is a mammalian cytokine.

In one embodiment, the present invention provides a process for producing the compositions of the invention comprising one or more of the following steps: i) expression of a polynucleotide encoding a polypeptide according to SEQ ID NOs: 2-22, and 24-25 in a host cell; ii) culturing the host cell to express the polypeptide as inclusion bodies; iii) washing the inclusion bodies; iv) solubilizing the polypeptide; v) refolding the polypeptide; and dialyzing the polypeptide into a therapeutically acceptable buffer.

### BRIEF DESCRIPTION OF THE SEVERAL VIEWS OF THE DRAWINGS

FIG. 1: Alignment of CT-1 protein sequences from various species. Amino acid sequences of cardiotrophin 1 from human mouse and rat. Identical amino acids are shaded. Cysteine residues in the human sequence and relative residues in the rodent sequences are in bold and underlined.

FIG. 2: CT-1 variants summary. Schematic depicting the relative amino acid sequence changes between wild type human CT-1 and the novel compositions constructed, expressed, purified and tested. Construct numbers and symbols used throughout this application to describe the constructs are listed. Wild type human CT-1 (FTV370) contains two cysteine residues, one at position 105 and one at position 178. Novel variants FTV380, FTV381 and FTV382 have either one or both cysteine residues mutated. Novel variants FTV383, FTV384 and FTV385 have both cysteines (relative to positions 105 and 178 in wild type CT-1) mutated and have additional cysteine residues either at the amino or carboxyl terminus or at both termini.

FIG. 3: Purification of wild-type and variants of human CT-1. Strategy used to purify the various CT-1 variant proteins: A.) flow diagram highlighting the steps of inclusion body refolding and final purification of CT-1. Step-yields for each stage of the process are listed in mg protein. B.) Ion exchange chromatography stage of the purification showing the separation of CT-1 from contaminating host cell proteins. UV (A280) chromatography trace is shown aligned with a coomassie stained SDS-PAGE depicting protein in each fraction collected. C.) Example coomassie-stained SDS-PAGE gel showing purity of final proteins. D.) Example HPLC SEC chromatography of the purified CT-1 proteins, depicting purity and homogeneity of the preparations. E.) HPLC-SEC with Area Under Curve analysis (AUC) showing purity and homogeneity of monomeric CT-1 polypeptide.

FIG. 4: Examples of cysteine modifications strategies. CT-1 protein and variants can be modified in a site specific manner by utilizing maleimide chemistry conjugation to the thiol groups of cysteine residues. B.) Examples of PEG modifications: single chain or branched chain PEG molecules can be used to modify the CT-1 protein and variants via maleimide-thiol conjugation. Small single chain PEG molecules of 12 and 24 PEG units or branched-chain PEG molecules of 3×12 PEG units were initially used. C.) Further examples of PEG modifications: large molecular weight single-chain or branched-chain PEG molecules such as the 30 KDa and 40 KDa PEGs depicted were also used to modify CT-1 variants with single cysteine/thiol groups. D.) CT-1 protein and variants can be dimerized or multimerized using flexible PEG linkers with two maleimide groups. The length of the PEG linker may vary depending on steric hindrance.

FIG. 5: Site specific PEGylation of novel CT-1 variants. Coomassie-stained SDS-PAGE depicting the molecular weight changes associated with site specific modification (PEGylation) of the CT-1 novel variant proteins. MM(PEG)₁₂ denotes a linear chain PEG with 12 units, MM(PEG)₂₄ denotes a linear chain PEG with 24 units and TMM(PEG)₁₂ denotes a branched chain PEG with 12 units/chain. A.) Protein FTV383, which has both naturally occurring cysteine residues replaced with serines and an additional cysteine residue at its amino terminus, can be PEGylated with single chain or branched chain molecules via thiol-maleimide conjugation albeit with low efficiency. In contrast FTV384, which has both naturally occurring cysteine residues replaced with serines and an additional cysteine residue at its carboxyl terminus, can be PEGylated with single chain or branched chain molecules with high efficiency. B). FTV384 is PEGylated extremely efficiently with almost no residual unmodified protein seen after a 30 minute reaction. C.) FTV380, which has the naturally occurring cysteine at position 105 replaced with serine, can be PEGylated at the remaining cysteine (178) albeit with low efficiency. In contrast FTV381, which retains the cysteine at position 105 and has cysteine 178 replaced, can be PEGylated to high efficiency. D.) Coomassie-stained SDS-PAGE displaying the site specific PEGylation of CT-1 variants FTV381 and FTV384 using large molecular weight PEG molecules as indicated. E.) Western bolt analysis using anti-CT-1 antibody of the site specific PEGylation of CT-1 variants FTV381 and FTV384 using large molecular weight PEG molecules as indicated.

FIG. 6: Site specific dimerization of the novel CT-1 variants. Coomassie-stained SDS-PAGE showing the dimerization of novel CT-1 variants. Construct numbers, PEG linkers and times of reaction are listed. Short, flexible linker PEG molecules with two maleimide active groups were used to dimerize the CT-1 variants (BM(PEG)₂ denotes a 2 PEG unit linker and BM(PEG)₃ denotes a 3 PEG unit linker). FTV383, which has an amino terminal cysteine residue, can be dimerized to a degree but the process is somewhat inefficient. In contrast FTV384, which has a carboxy terminal cysteine, can be efficiently dimerized via its cysteine using the PEG linker to form a protein of 44 KDa. B.) FPLC SEC chromatogram and SDS-PAGE showing purification of dimerized FTV384 protein.

FIG. 7: Activity Assessments of novel CT-1 variants by Stat3 phosphorylation assay. A titration of novel CT-1 variants or wild type protein concentration was used to treat C2C12 cells for 20 minutes after which cells were harvested and lysed. Lysates were western blotted for total and phosphorylated Stat3 and actin as a loading control. A.) Example western blots showing constant blot intensity for total Stat3 and actin and a titration of phosphorylated Stat3 when cells are treated with the concentrations of CT-1 or variants as indicated. B.) Densitometry was performed on the western blots and values for phosphorylated Stat3 were normalized to total Stat3 to produce dose response curves. EC50 values were generated from the curves as indicated.

FIG. 8: Novel CT-1 Variants with improved activity. Stat3 phosphorylation activity assays were performed in triplicate, comparing wild-type CT-1 and FTV380, a novel CT-1 variant with cysteine 105 mutated to serine. EC50 values are indicated.

FIG. 9: In vitro activity assessment and comparison of novel CT-1 variants. Stat3 phosphorylation activity assays were completed as previously described and mean EC50 values from multiple experiments plotted with standard deviation. CT-1 variants and PEG or dimer modifications are indicated as is mean EC50 A.) CT-1 variants prior to modification (PEGylation or dimerization) were compared for specific activity. B.) Mean specific activities displayed as EC50 comparing wild-type CT-1 protein with variant FTV380 pre and post modification of this variant by PEGylation. C.) Mean specific activities displayed as EC50 comparing wild-type CT-1 protein with variant FTV381 pre and post modification of this variant by PEGylation. D.) Mean specific activities displayed as EC50 comparing wild-type CT-1 protein with variant FTV383 pre and post modification of this variant by PEGylation. E.) Mean specific activities displayed as EC50 comparing wild-type CT-1 protein with variant FTV384 pre and post modification of this variant by PEGylation and dimerization.

FIG. 10: Endotoxin assessments of therapeutic CT-1 polypeptide. Table describing the results of kinetic LAL assays to measure the endotoxin content of two independent batches of therapeutic CT-1 polypeptide. Endotoxin units (EU) are expressed as volume and mg therapeutic protein.

FIG. 11: N-terminal sequencing of therapeutic CT-1 polypeptide. Table showing results of N-terminal sequencing by Edman degradation and mass-spectrometry. Amino acid sequence indicates the effective removal of the N-terminal methionine residue to result in a homogeneous protein product.

FIG. 12: Total mass and enzymatic digest peptide analysis of therapeutic CT-1 polypeptide. Mass spectrometry analysis of whole protein and trypsin digest peptides in order to analyse post-translational integrity of the therapeutic CT-1 polypeptide. A.) Whole protein Mass spectrometry analysis shows the CT-1 polypeptide to be 21.094 KDa in mass. B.) LC-MS trace of CT-1 trypsin digest showing retention of peptides. C.) Predicted peptide sequence resulted in 96.5% coverage of the protein indicating no post-translational modifications.

FIG. 13: Stability assessments of commercially available forms of CT-1. Stability assessment of various forms and formulations of commercially available CT-1 polypeptide. Protein forms, formulations, temperatures and timepoints of stability assessments as indicated. SDS-PAGE western blot assessment of residual protein in solution at times indicated.

FIG. 14: Stability assessments of therapeutic CT-1 polypeptide. Stability assessment of therapeutic CT-1 polypeptide. Protein forms, formulations, temperatures and timepoints of stability assessments as indicated. A.) Coomassie-stained SDS-PAGE western blot assessment of residual protein in solution at times indicated. B.) Densitometry analysis of coomassie signal normalized to starting value. Proportion of retained protein as indicated.

FIG. 15: In vitro activity assessment of therapeutic CT-1 polypeptide. In vitro assessment of therapeutic CT-1 polypeptide activity using Stat3 phosphorylation assay. A.) Example data showing triplicate Western-blot analysis of phosphorylated Stat3 resulting from CT-1 treatment titration. Total Stat3 and actin loading controls as indicated. B.) Western-blot densitometry quantification and curve plotting as dose response results in EC50 values for two batched of therapeutic CT-1 polypeptide (FTV370) and a less active mutant of CT-1 (FTV382). Mean EC50 values for 8 independent titration studies for therapeutic CT-1 polypeptide and standard deviations are tabulated.

### BRIEF DESCRIPTION OF THE SEQUENCE IDENTIFIERS

SEQ ID NO: 1 sets forth a cDNA sequence encoding human CT-1.

SEQ ID NO: 2 sets forth the amino acid sequence of the human CT-1 polypeptide encoded by SEQ ID NO: 1.

SEQ ID NO: 3 sets forth the amino acid sequence of a modified human CT-1 polypeptide having a serine at amino acid position 105.

SEQ ID NO: 4 sets forth the amino acid sequence of a modified human CT-1 polypeptide having a serine at amino acid position 178.

SEQ ID NO: 5 sets forth the amino acid sequence of a modified human CT-1 polypeptide having a serine at amino acid positions 105 and 178.

SEQ ID NO: 6 sets forth the amino acid sequence of a modified human CT-1 polypeptide having a serine at amino acid positions 106 and 179 and a cysteine at amino acid position 2.

SEQ ID NO: 7 sets forth the amino acid sequence of a modified human CT-1 polypeptide having a serine at amino acid positions 105 and 178 and a cysteine at amino acid position 202.

SEQ ID NO: 8 sets forth the amino acid sequence of a modified human CT-1 polypeptide having a serine at amino acid positions 106 and 179 and a cysteine at amino acid positions 2 and 203.

SEQ ID NO: 9 sets forth the amino acid sequence of a modified human CT-1 polypeptide, lacking an N-terminal methionine and having serine at the amino acid position corresponding to position 105 of SEQ ID NO: 2.

SEQ ID NO: 10 sets forth the amino acid sequence of a modified human CT-1 polypeptide, lacking an N-terminal methionine and having a serine at the amino acid position corresponding to position 178 of SEQ ID NO: 2.

SEQ ID NO: 11 sets forth the amino acid sequence of a modified human CT-1 polypeptide, lacking an N-terminal methionine and having serine at the amino acid positions corresponding to position 105 and 178 of SEQ ID NO: 2.

SEQ ID NO: 12 sets forth the amino acid sequence of a modified human CT-1 polypeptide, lacking an N-terminal methionine and having a cysteine at the amino acid position corresponding to position 202 of SEQ ID NO: 2.

SEQ ID NO: 13 sets forth the amino acid sequence of a modified human CT-1 polypeptide, lacking an N-terminal methionine, having a serine at amino acid position 105 and having a cysteine at the amino acid position corresponding to position 202, of SEQ ID NO: 2.

SEQ ID NO: 14 sets forth the amino acid sequence of a modified human CT-1 polypeptide, lacking an N-terminal methionine, having a serine at amino acid position 178 and having a cysteine at the amino acid position corresponding to position 202, of SEQ ID NO: 2.

SEQ ID NO: 15 sets forth the amino acid sequence of a modified human CT-1 polypeptide, lacking an N-terminal methionine, having a serine at amino acid positions 105 and 178 and having a cysteine at the amino acid position corresponding to position 202, of SEQ ID NO: 2.

SEQ ID NO: 16 sets forth the amino acid sequence of a modified human CT-1 polypeptide, having an N-terminal cysteine and having a serine at amino acid position 105.

SEQ ID NO: 17 sets forth the amino acid sequence of a modified human CT-1 polypeptide, having an N-terminal cysteine and having a serine at amino acid position 178.

SEQ ID NO: 18 sets forth the amino acid sequence of a modified human CT-1 polypeptide, having an N-terminal cysteine and having a serine at amino acid positions 105 and 178.

SEQ ID NO: 19 sets forth the amino acid sequence of a modified human CT-1 polypeptide, having an N-terminal cysteine and having a cysteine at the amino acid position 202.

SEQ ID NO: 20 sets forth the amino acid sequence of a modified human CT-1 polypeptide, having an N-terminal cysteine, having a serine at amino acid positions 105, and having a cysteine at the amino acid position 202.

SEQ ID NO: 21 sets forth the amino acid sequence of a modified human CT-1 polypeptide, having an N-terminal cysteine, having a serine at amino acid position 178, and having a cysteine at the amino acid position 202.

SEQ ID NO: 22 sets forth the amino acid sequence of a modified human CT-1 polypeptide, having an N-terminal cysteine, having a serine at amino acid positions 105 and 178, and having a cysteine at the amino acid position 202.

SEQ ID NO: 23 sets forth the amino acid sequence of a modified human CT-1 polypeptide.

SEQ ID NO: 24 sets forth the amino acid sequence of a human CT-1 polypeptide.

SEQ ID NO: 25 sets forth the amino acid sequence of a human CT-1 polypeptide of SEQ ID NO: 24, lacking an N-terminal methionine.

### DETAILED DESCRIPTION

### A. Overview

The invention provides polypeptides having CT-1 biological activity that are suitable for therapeutic use. In some aspects, the polypeptides of the invention include novel CT-1 polypeptides, variants, oligomers, and chemically modified derivatives thereof possessing CT-1 biological activity. The invention also provides polypeptides having the amino acid sequence of wild type CT-1, wherein the polypeptides are produced by the methods of the invention and possess improved stability, purity, or endotoxin or aggregation profiles. In addition to methods for producing wild-type and variant CT-1 polypeptides that are suitable for therapeutic use, the invention also provides compositions of wild-type or variant CT-1 polypeptides, wherein the compositions have improved biologic drug-like properties such as a prolonged plasma half-life in mammals, and especially in humans, enhanced stability, solubility, production, and formulation, and therapeutic uses for such CT-1 compositions. Therapeutic uses for the CT-1 compositions of the invention include, for example, promoting tissue growth, regeneration, repair, protection or maintenance.

In particular aspects of the invention, the novel variants of the invention include modifications that, in part, enhance the pharmacokinetic properties of the polypeptide, such as plasma-half life, without causing deleterious effects on the desirable properties of the polypeptide, including the polypeptide's biological activity, stability, purity, and solubility. For example and without limitation, in some embodiments of the invention the novel variants include one or more amino acid additions, deletions, or substitutions to facilitate site-specific modifications of the polypeptide. In some embodiments, the novel variants are modified by site-specific modification to increase the pharmacokinetic and drug-like properties of the polypeptides. In one embodiment, the novel variants are PEGylated by site specific modification to increase the molecular weight and plasma half-life of the protein for therapeutic use.

The invention also provides methods of producing polypeptides possessing CT-1 biological activity, including polypeptides having the amino acid sequence of wild-type CT-1 polypeptide, as well as the novel variants described herein, wherein the resulting polypeptides have enhanced therapeutic properties (i.e., as compared to CT-1 polypeptides not produced by the methods of the invention). In some embodiments, the polypeptides produced by the methods of the invention exhibit improved solubility, stability (such as increased stability against degradation and aggregation), and purity over CT-1 polypeptides not prepared by the methods of the invention.

Each and every one of the CT-1 polypeptides disclosed herein represents an aspect of the invention, as do nucleic acids encoding CT-1 polypeptides, a vector comprising such nucleic acids, an expression system such as a host cell comprising such a vector, a composition comprising a CT-1 polypeptide, a CT-1 polypeptide of the invention for use in a method of treatment of the human or animal body, use of a CT-1 polypeptide in the manufacture of a medicament, such as a medicament for promoting tissue growth and/or regeneration, a method of making the CT-1 polypeptide and other compositions, methods and uses as disclosed herein.

The practice of the invention will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); and Harlow and Lane, Antibodies, (Cold Spring Harbor Laboratory Press, Cold Spring Harbor, N.Y., 1998).

“Commercially available chemicals” may be obtained from standard commercial sources including, but not limited to Quanta Biodesign (Powell, Ohio), Iris Biotech (GmbH), Nanocs, Inc. (Ney York, N.Y.), Nektar (San Carlos, Calif.), Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hanover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

Methods known to one of ordinary skill in the art may be identified through various reference books and databases. Suitable reference books and treatises that detail the synthesis of reactants useful in the preparation of compounds and compound conjugates described herein, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions,” 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of conjugate compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R.V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

All publications, patents and patent applications cited herein are hereby incorporated by reference in their entirety.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of methods and materials are described herein. For the purposes of the present invention, the following terms are defined below.

The articles “a,” “an,” and “the” are used herein to refer to one or to more than one (i.e., to at least one) of the grammatical object of the article. By way of example, “an element” means one element or more than one element.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the terms “polypeptide,” “peptide,” and “protein” are used interchangeably, unless specified to the contrary, and according to conventional meaning, i.e., as a sequence of amino acids linked by peptide bonds or modified peptide bonds. Polypeptides are not limited to a specific length, e.g., they may comprise a full length protein sequence or a fragment of a full length protein, addition, deletion, modification, or substitution of one or more amino acids at the N-terminus, internal portion, and/or C-terminus of the protein, and may include post-translational modifications of the polypeptide, for example, pegylations, glycosylations, acetylations, phosphorylations and the like, as well as other modifications known in the art, both naturally occurring and non-naturally occurring. Polypeptides of the invention may be prepared using any of a variety of well known recombinant and/or synthetic techniques, illustrative examples of which are further discussed below.

As used herein, the terms “wild type CT-1” and “wild type CT-1 polypeptide” refer to a polypeptide having a polypeptide sequence as set forth in SEQ ID No: 2. As used herein, the term “CT-1 polypeptide,” refers to a CT-1 protein having a polypeptide sequence as set forth in SEQ ID No: 2, and variants, analogs, derivatives, and modifications thereof, including, without limitation, any number of amino acid additions, deletions, modifications, or substitutions at the N-terminus, internal portion, and/or C-terminus of the protein, see, e.g., SEQ ID NOs: 3-25.

The terms “CT-1 variant” or “modified CT-1 polypeptide” refers to a modified or engineered CT-1 polypeptide that is distinguished from wild type CT-1 polypeptide by the modification, addition, deletion, or substitution of at least one amino acid residue, as discussed elsewhere herein and as understood in the art. In certain embodiments, a CT-1 variant is distinguished from a reference polypeptide by one or more amino acid substitutions (e.g., 1, 2, 3, 4, 5 or more substitutions), which may be conservative or non-conservative. In particular embodiments, a CT-1 variant has a sequence that is at least about 70%, more preferably about 80%, 85%, 90%, 91%, 92%, 93%, 94%, 95%, 96%, 97%, 98%, or 99%, identical to a wild type CT-1 sequence. Identity may be assessed over at least about 5, 10, 25, 50, 100, 150, or 175, or more contiguous amino acids, or may be assessed over the full length of the sequence. Methods for determining % identity or % homology are known in the art and any suitable method may be employed for this purpose. Variants of the polypeptides of the invention include glycosylated forms, aggregative conjugates with other molecules, and covalent conjugates with unrelated chemical moieties (e.g., PEGylated molecules). In particular embodiments variants also include allelic variants, species variants, and muteins. In certain embodiments, truncations or deletions of regions which do not affect functional activity of the proteins are also contemplated as variants. Illustrative examples of CT-1 polypeptides and CT-1 variants are set forth in SEQ ID NOs: 2-25.

As used herein, a polypeptide composition having “CT-1 activity,” “CT-1 biological activity”, “naturally occurring CT-1 activity,” or “unmodified CT-1 activity,” refers to a polypeptide having at least one biological activity of wild type CT-1 polypeptide, where such wild type CT-1 polypeptide is not produced, prepared, purified, or formulated according to the methods of the invention. A polypeptide has at least one biological activity of wild type CT-1 polypeptide if the polypeptide of interest carries out a similar function in the cell, e.g., a similar enzymatic reaction or a similar signaling mechanism, or generates a similar physiological response as wild type CT-1 not produced by the methods of the invention. For example, a polypeptide of the invention has CT-1 activity if both wild type CT-1 and the polypeptide provided by the invention are involved in similar activation of intracellular pathways, such as JAK/Stat3, PI3-kinase/AKT, and/or ERK signaling activation.

A polypeptide of the invention having CT-1 activity may generate a physiological response that is at least 100%, at least 90%, at least 80%, at least 70%, at least 60%, at least 50%, at least 40%, at least 30%, at least 20%, at least 10%, or at least 5% of the physiological response generated by the corresponding naturally occurring CT-1 polypeptide having the amino acid sequence set forth in SEQ ID NO: 2.

As used herein, the terms “promoting,” “enhancing,” “stimulating,” or “increasing” generally refer to the ability of a CT-1 composition of the invention to produce or cause a greater physiological response (i.e., measurable downstream effect), as compared to the response caused by either vehicle (or the absence of CT-1) or a control molecule/composition. One such measurable physiological response includes, without limitation, the ability to cause an increase in activation of the JAK/STAT, PI3-Kinase, and/or ERK pathways compared to level of activation of the JAK/STAT, PI3-Kinase, and/or ERK pathways in the absence of administration of the CT-1 composition or compared to the activation caused by vehicle or control composition. For example, the physiological response may be increased by at least 5%, 10%, 20%, 30%, 40%, 50%, 60%, 70%, 80%, 90%, 100%, 125%, 150% 175%, 200%, or greater. In another non-limiting example, activation of the JAK/STAT pathway in response to administration of a CT-1 composition of the invention may be increased by at least 5%, 10%, 20%, 30%, 40%, 50%, 60%, 70%, 80%, 90%, 100%, 125%, 150%, 175%, 200%, or greater, compared to the level of activation of the JAK/STAT pathway caused by either vehicle or a control molecule/composition. An “increased” or “enhanced” response is typically a “statistically significant” response, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response produced by vehicle or a control composition.

As used herein, the terms “retaining” or “maintaining,” generally refer to the ability of a CT-1 polypeptide of the invention to produce or cause a physiological response (i.e., measurable downstream effect) that is of a similar nature to the response caused by a CT-1 polypeptide of the naturally occurring CT-1 amino acid or nucleic acid sequence. For example, the CT-1 polypeptides of the invention exhibit CT-1 biological activity, and thus retain CT-1 activity. The compositions of the invention also produce a physiological response, such as activation of JAK/STAT, PI3-Kinase, and/or ERK pathways, that is of a similar nature to the response caused by a naturally occurring CT-1 polypeptide. A CT-1 composition of the invention that elicits a similar physiological response may elicit a physiological response that is at least 5%, at least 10%, at least 15%, at least 20%, at least 25%, at least 30%, at least 40%, at least 50%, at least 60%, at least 70%, at least 75%, at least 80%, at least 85%, at least 90%, at least 95% or about 100% of the level of physiological response elicited by a composition comprising a naturally occurring CT-1 amino acid or nucleic acid sequence.

As used herein, the terms “decrease” or “lower,” or “lessen,” or “reduce,” or “abate” refers generally to the ability of a CT-1 polypeptide of the invention to produce or cause a lesser physiological response (i.e., downstream effects), as compared to the response caused by either vehicle or a control molecule/composition, e.g., decreased apoptosis. In one embodiment, the decrease can be a decrease in activation of the JAK/STAT, PI3-Kinase, and/or ERK pathways. A “decrease” or “reduced” response is typically a “statistically significant” response, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response produced by vehicle (the absence of an agent) or a control composition.

As used herein, the term “naturally occurring”, refers to a polypeptide or polynucleotide sequence that can be found in nature. For example, a naturally occurring polypeptide or polynucleotide sequence would be one that is present in an organism, and can be isolated from the organism, and which has not been intentionally modified by man in the laboratory. The term “wild-type” is often used interchangeably with the term “naturally occurring.”

As used herein, the term “isolated” means material that is substantially or essentially free from components that normally accompany it in its native state. For example, an “isolated polynucleotide,” as used herein, refers to a polynucleotide that has been purified from the sequences which flank it in a naturally-occurring state, e.g., a DNA fragment that has been removed from the sequences that are normally adjacent to the fragment. Alternatively, an “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

As used herein, the term “obtained from” means that a sample such as, for example, a polynucleotide or polypeptide is isolated from, or derived from, a particular source, such as a recombinant host cell. In another embodiment, the term “obtained from” refers to a cell isolated from or derived from a source such as an in vivo tissue or organ.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other (see for example, Usman and McSwiggen, supra; Eckstein et al., International PCT Publication No. WO 92/07065; Usman et al., International PCT Publication No. WO 93/15187; Uhlman & Peyman, supra). There are several examples of modified nucleic acid bases known in the art as summarized by Limbach et al., (1994, Nucleic Acids Res. 22, 2183-2196).

As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

The terms “amino-terminal” (or “N-terminal”) and “carboxyl-terminal” (or “C-terminal”) are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “corresponding to”, when applied to positions of amino acid residues in sequences, means corresponding positions in a plurality of sequences when the sequences are optimally aligned or in a two way comparison, to the amino acid position of a reference sequence.

As used herein, the term “good manufacturing practice (GMP)” refers to the control and management of manufacturing, and quality control testing, of foods, pharmaceutical products, and medical devices. GMP does not necessarily rely on sampling, but instead relies on documentation of every aspect of the process, activities, and operations involved with drug and medical device manufacture. If the documentation showing how the product was made and tested (which enables traceability and, in the event of future problems, recall from the market) is not correct and in order, then the product does not meet the required specification and is considered contaminated (i.e., adulterated in the US). Additionally, GMP typically requires that all manufacturing and testing equipment has been qualified as suitable for use, and that all operational methodologies and procedures (e.g., manufacturing, cleaning, and analytical testing) utilized in the drug manufacturing process have been validated according to predetermined specifications to demonstrate that they can perform their purported function(s). In the US, the phrase “current good manufacturing practice” appears in 501(B) of the 1938 Food, Drug, and Cosmetic Act (21 U.S.C. §351).

### C. Polypeptides

The invention provides, in part, polypeptides comprising an amino acid sequence selected from the group consisting of SEQ ID NOs: 2-25, and analogs thereof, having at least one biological activity of CT-1. The invention also provides polypeptides, and compositions thereof, comprising an amino acid sequence set forth in SEQ ID NO: 2-25, wherein the polypeptide is produced by the methods of the invention to possess improved stability, purity, and/or endotoxin and aggregation profiles.

Our sequence analysis of CT-1 polypeptide suggests a lack of post-translational modifications such as glycosylation and lipidation, therefore production of the protein for therapeutic use could utilize either common mammalian or prokaryotic expression systems. The soluble nature of the protein produced by the methods of the present invention and its predicted isoelectric point of pl 9.1 are also advantageous to production and formulation required for therapeutic development. However, CT-1 polypeptide has a low molecular weight (21.2 KDa) which may result in rapid clearance from the body leading to a short systemic half-life and bioavailability on in vivo delivery. Thus, in other embodiments, the invention also provides compositions and formulations of CT-1 polypeptides and/or polynucleotides suitable for therapeutic use.

In one embodiment, the CT-1 polypeptides of the invention comprise an initial N-terminal methionine. In particular embodiments, the CT-1 polypeptides of the invention do not comprise an initial N-terminal methionine. In various embodiments, an amino acid is added between the N-terminal methionine encoded by the start codon and the adjacent amino acid residue in the CT-1 polypeptide, e.g., serine. The added amino acid can be any amino acid or a particular amino acid. In one embodiment, the amino acid added between the N-terminal methionine encoded by the start codon and the adjacent amino acid residue in the CT-1 polypeptide is selected from the group consisting of cysteine, lysine, histidine, asparagine, aspartate, glutamate, alanine, glycine, threonine, serine, and valine.

In other particular embodiments, the CT-1 polypeptides of the invention do not comprise an initial N-terminal methionine but contain one or amino acids added the N-terminus of the polypeptide. In various embodiments, the amino acid added to the N-terminus of the CT-1 polypeptide is selected from the group consisting of cysteine, lysine, histidine, asparagine, aspartate, glutamate, alanine, glycine, threonine, serine, and valine. It is contemplated that the added amino acid may be added 1, 2, 3, 4, 5, 6, 7, 8, 9, or 10 amino acids away from the N-terminus or to the N-terminal position (position 1).

In the polypeptides of the invention, one or more cysteine residues present in the naturally-occurring CT-1 polypeptide are substituted, modified, or deleted. In some embodiments, the cysteine residue at position 105 of any one of SEQ ID NOs: 2-5, 7, and 9-22 or at position 106 of any one of SEQ ID NOs: 6, 8, and 23 is replaced by serine. In other embodiments, the cysteine residue at position 178 of any one of SEQ ID NOs: 2-5, 7, and 9-22 or at position 179 of any one of SEQ ID NOs: 6, 8, and 23 is replaced by serine. Optionally, both cysteine residues, i.e., at position 105 and 178 of any one of SEQ ID NOs: 2-5, 7, and 9-22 or at position 106 and 179 of any one of SEQ ID NOs: 6, 8, and 23, are replaced with serine residues.

In particular embodiments, a CT-1 polypeptide corresponds to the amino acid set forth in SEQ ID NO: 23, wherein the amino acid (Xaa) at position 1 of SEQ ID NO: 23 is absent or methionine, wherein the amino acid at position 2 of SEQ ID NO: 23 is absent or any amino acid, and wherein the amino acid at position 203 of SEQ ID NO: 23 is absent or any amino acid. In related embodiments, the CT-1 polypeptides as set forth in SEQ ID NO: 23 comprise one or more substituted, modified, or deleted cysteine residues, e.g., at positions 106, 179, 203 of SEQ ID NO: 23.

In certain embodiments, a CT-1 polypeptide corresponds to the amino acid set forth in SEQ ID NO: 23, wherein the amino acid (Xaa) at position 1 of SEQ ID NO: 23 is absent or methionine, wherein the amino acid at position 2 of SEQ ID NO: 23 is absent or cysteine, lysine, histidine, asparagines, aspartate, glutamate, alanine, glycine, threonine, serine, and valine, and wherein the amino acid at position 203 of SEQ ID NO: 23 is absent or cysteine. In related embodiments, the CT-1 polypeptides as set forth in SEQ ID NO: 23 comprise one or more substituted, modified, or deleted cysteine residues, e.g., at positions 106, 179, 203 of SEQ ID NO: 23.

In certain particular embodiments, a CT-1 polypeptide corresponds to the amino acid set forth in SEQ ID NO: 23, wherein the amino acid (Xaa) at position 1 of SEQ ID NO: 23 is absent or methionine, wherein the amino acid at position 2 of SEQ ID NO: 23 is cysteine, alanine, glycine, serine, threonine, serine, or valine, cysteine, lysine, histidine, asparagine, aspartate, glutamate, alanine, glycine, threonine, serine, and valine, and wherein the amino acid at position 203 of SEQ ID NO: 23 is absent or cysteine. In related embodiments, the CT-1 polypeptides as set forth in SEQ ID NO: 23 comprise one or more substituted, modified, or deleted cysteine residues, e.g., at positions 106, 179, 203 of SEQ ID NO: 23.

In certain particular embodiments, a CT-1 polypeptide corresponds to the amino acid set forth in SEQ ID NO: 23, wherein the amino acid (Xaa) at position 1 of SEQ ID NO: 23 is absent, wherein the amino acid at position 2 of SEQ ID NO: 23 is cysteine and wherein the amino acid at position 203 of SEQ ID NO: 23 is absent or cysteine. In related embodiments, the CT-1 polypeptides as set forth in SEQ ID NO: 23 comprise one or more substituted, modified, or deleted cysteine residues, e.g., at positions 106, 179, 203 of SEQ ID NO: 23.

In more particular embodiments, one or more cysteines of any one of SEQ ID NOs: 3-25 may be substituted, modified, or deleted. In certain embodiments, one or more cysteines of any one of SEQ ID NOs: 3-25 are replaced with serine.

In other embodiments, the invention contemplates CT-1 polypeptides comprising addition, deletion, substitution, or modification of 1, 2, 3, 4, 5, 6, 7, 8, 9, 10 or more amino acids of a CT-1 polypeptide compared to the naturally occurring CT-1 polypeptide sequence. In one embodiment, CT-1 polypeptides comprise the addition of 1, 2, 3, 4, or 5 or more amino acids to the N-terminus compared to the naturally occurring CT-1 polypeptide sequence. In some embodiments, CT-1 polypeptides comprise the addition of 1, 2, 3, 4, or 5 or more amino acids to the N-terminus of a naturally occurring CT-1 polypeptide sequence that does not have an N-terminal methionine. In further embodiments, the present invention contemplates CT-1 polypeptides comprising the addition of 1, 2, 3, 4, 5, 6, 7, 8, 9, 10 or more amino acids to the C-terminus compared to the naturally occurring CT-1 polypeptide sequence. It would be appreciated by those having ordinary skill in the art that any number of or all of the amino acids added to the N-terminus can be modified, such as, for example, glycosylation or PEGylation.

The invention also provides novel CT-1 polypeptides and methods of producing the same, comprising an amino acid sequence selected from the group consisting of SEQ ID NOs: 3-25, and optionally comprising an initial N-terminal methionine and/or an additional amino acid, wherein the polypeptide is chemically modified.

In some embodiments, the CT-1 polypeptides of the invention are conjugated to a biocompatible polymer, and in particular embodiments are, PEGylated. The polypeptides of the invention may comprise about 5, about 4, about 3, about 2, about 1, or about 0.5 PEG moieties per one molecule of the polypeptide.

In certain embodiments, one or more cysteines of any one of SEQ ID NOs: 3-25 may be conjugated to a biocompatible polymer, e.g., PEG. The polypeptides of the invention retain CT-1 biological activity and in some embodiments, have increased CT-1 biological activity. Particular CT-1 polypeptides of the invention have enhanced pharmacokinetic properties, such as extended plasma half-life, making them well-suited for therapeutic uses, and may additionally exhibit improved solubility and/or stability over wild-type CT-1 protein. In particular embodiments, the inventive CT-1 polypeptides promote tissue growth, protection, and regeneration. The invention also provides polynucleotides encoding such novel CT-1 polypeptides, and compositions comprising novel CT-1 polypeptides and/or polynucleotides.

The enhanced pharmacokinetic properties of a wild-type CT-1 polypeptide, as well as the novel variants described herein are relative to CT-1 polypeptides not produced by the methods of the invention.

In one embodiment, the half-life of the inventive polypeptides are increased at least 1.2 fold, at least 1.5 fold, at least 2 fold, at least 2.5 fold, at least 3 fold, at least 3.5 fold, at least 4 fold, at least 4.5 fold, or at least 5 fold compared to a non-modified polypeptide (e.g., a naturally occurring polypeptide), and/or CT-1 polypeptides that are not produced according to the methods of the invention.

In particular embodiments, the inventive polypeptides are stable for at least 6 hours, at least 7 hours, at least 8 hours, at least 9 hours, at least 10 hours, at least 11 hours, at least 12 hours, at least 15 hours, at least 18 hours, at least 21 hours, at least 24 hours, at least 48 hours, or more at about 37° C. compared to a non-modified polypeptide (e.g., a naturally occurring polypeptide), and/or CT-1 polypeptides that are not produced accorinding to the methods of the invention.

As used herein, the terms “CT-1 variant”, “CT-1 variant polypeptide”, “modified CT-1 polypeptide,” “modified or engineered CT-1 polypeptide,” and “engineered CT-1 polypeptide,” are used interchangeably and refer to a CT-1 polypeptide, biologically active fragments or variants thereof, or homolog, paralog, or ortholog thereof that comprises one or more amino acid mutations, additions, deletions, or substitutions compared to wild type CT-1 protein. In particular embodiments of the invention, modified CT-1 polypeptides comprise one or more amino acid mutations, additions, deletions, and/or substitutions that also result in a CT-1 polypeptide that retains CT-1 biological activity. In particular embodiments, the CT-1 polypeptide variant is further modified by PEGylation, particularly site-specific PEGylation, and retains CT-1 activity. In some embodiments, CT-1 polypeptides are conjugated together, such as by PEG linkage or other biocompatible polymer, to form CT-1 oligomers, and thus the invention provides CT-1 multimers. Preferably, CT-1 polypeptides of the invention retain at least 100%, at least 90%, at least 80%, at least 70%, at least 60%, at least 50%, at least 40%, at least 30%, at least 20%, at least 10%, or at least 5% activity of the naturally occurring CT-1 activity. Illustrative examples of CT-1 polypeptide variants are set forth in SEQ ID NOs: 3-25.

In the context of the invention, a polypeptide, a biologically active fragment or variant thereof, or homolog, paralog, or ortholog thereof, is considered to have at least substantially the same activity as the wild-type protein when it exhibits about 10%, 20%, 30%, 40% or 50% of the activity of the wild-type protein, preferably at least 50%, at least 55%, at least 60%, at least 65%, at least 70%, at least 75%, or at least 80% of the activity of the wild type protein. In particular embodiments, the polypeptide, a biologically active fragment or variant thereof, or homolog, paralog, or ortholog thereof, exhibits at least 70%, at least 80%, at least 90%, at least 95% or about 100% of the activity of the wild-type protein. In certain embodiments, an activity greater than wild type activity may be achieved. Activity of a CT-1 polypeptide, a biologically active fragment or variant thereof, or homolog, paralog, or ortholog thereof, for example, can be determined by measuring its ability to mimic wild-type CT-1 biological activity by, for example, activating the JAK/STAT pathway, and comparing the ability to the activity of a wild type protein. Methods of measuring CT-1 biological activity are known in the art.

As used herein, the term “biologically active fragment,” as applied to fragments of a reference polynucleotide or polypeptide sequence, refers to a fragment of a modified CT-1 polypeptide that has at least about 5, 10, 15, 20, 25, 30, 40, 50, 55, 60, 65, 70, 75, 80, 85, 90, 95, 96, 97, 98, 99, 100, 110, 120, 150, 200, 300, 400, 500, 600, 700, 800, 900, or 1000% or more of the biological activity of a CT-1 reference sequence, such as its biological activity to promote cell growth, proliferation and protection. Some embodiments of the present invention contemplate, in part, biologically active fragments of a modified CT-1 polypeptide of at least about 20, 50, 100, 150, 175, or 199, contiguous amino acid residues in length or polynucleotide sequences encoding the same, including all integers in between, which comprise or encode a polypeptide having the biological activity of a reference CT-1 polypeptide, e.g., a naturally occurring CT-1 polypeptide.

In various embodiments, the invention contemplates CT-1 polypeptide fragments that retain biological activity. Exemplary fragments include, but are not limited to polypeptides comprising at least 160, 161, 162, 163, 164, 165, 166, 167, 168, 169, 170, 171, 172, 173, 174, 175, 176, 177, 178, 179, 180, 181, 182, 183, 184, 185, 286, 187, 188, 189, 190, 191, 192, 193, 194, 195, 196, 197, 198, 199, 200, or 201, contiguous amino acids identical to the amino acid sequence set forth in SEQ ID NO: 2. Polypeptide fragments may further comprise one or more of: a Cys and/or a Lys at a non-naturally occurring amino acid position in SEQ ID NO: 2; at least one deletion of a naturally occurring amino acid of SEQ ID NO: 2, e.g., Cys 105 or Cys 178; at least one substitution of a naturally occurring amino acid of SEQ ID NO: 2, e.g., Cys 105 or Cys 178 with another amino acid or at least one modification of a naturally occurring amino acid of SEQ ID NO: 2, e.g., Cys 105 or Cys 178. In certain embodiments, Cys 105 and Cys 178 are substituted with glycine (G), serine (S), alanine (A), threonine (T), leucine (L), isoleucine (I), or arginine (R).

In particular embodiments, the polypeptides may comprise at least 160, 161, 162, 163, 164, 165, 166, 167, 168, 169, 170, 171, 172, 173, 174, 175, 176, 177, 178, 179, 180, 181, 182, 183, 184, 185, 286, 187, 188, 189, 190, 191, 192, 193, 194, 195, 196, 197, 198, 199, 200, or 201 non-contiguous amino acids identical to the amino acid sequence set forth in SEQ ID NO: 2, wherein the non-contiguous number of amino acids is 1, 2, 3, 4, 5, 6, 7, 8, 9, or amino acids.

In certain embodiments, the Cys at the non-naturally occurring amino acid position in SEQ ID NO: 2 is within about 1, 2, 3, 4, 5, 6, 7, 8, 9, or amino acids of the N- or C-terminus of the polypeptide.

The invention further contemplates, in particular embodiments, that compositions of the novel polypeptides are at least 75%, at least 80%, at least 85%, at least 90%, at least 95%, at least 96%, at least 97%, at least 98%, at least 99%, or 100% percent pure by weight with respect to the polypeptide.

### Chemically Modified CT-1 Derivatives.

Because CT-1 has a low molecular weight (21.2 KDa) which may be associated with rapid clearance from the body, and corresponding short systemic half-life and bioavailability on in vivo delivery, formulations or compositions of CT-1 that decrease clearance and/or absorption rates are particularly desirable. The invention thus provides CT-1 variants that are chemically modified to provide advantages for therapeutic use.

Methods of extending the systemic half-life and bioavailability of therapeutics are known in the art and include the use of slow release formulations, protein fusions to increase molecular weight and/or cellular uptake and cycling of administered molecules. In the particular case of CT-1 therapeutic development, the addition of a large modification or the dimerization/multimerization of the protein may result in significantly reduced receptor binding and therefore signaling activity.

The invention provides site-specific modification of the CT-1 proteins of the invention to mitigate potential deleterious effects associated with modifications. Through site-specific modification, amino acid residues that do not form part of an active site or binding site of a protein of interest can be altered to provide a preferred site within the protein for chemical modification, such as PEGylation and/or multimerization. Site-specific modification requires identifying regions of a protein, or more specifically amino acid residues that can be utilized for protein modification without substantially affecting bioactivity. In some cases it is necessary to substitute, add, or remove an amino acid of the natural amino acid sequence of a polypeptide to incorporate residues into sites or regions of a protein to create a site for chemical modification. In other cases, the removal of specific amino acid residue chemistries from regions can be conducted to prevent modification at undesirable sites.

In one aspect, the invention provides an analysis of the CT-1 amino acid sequence and structure to identify regions and residues that may be used for site-specific modification of the CT-1 polypeptides described herein. Exemplary embodiments of the invention provide for the identification of novel variants of CT-1 in which specific amino acid residues have been changed to allow for site specific modification of the protein, such as conjugation of polymers at specific sites of the protein, while retaining biological activity of this signaling molecule. Further, particular embodiments of the invention describe the identification of CT-1 variants with novel amino acid compositions with increased biological activity in comparison to the wild type protein. In addition, methods for site-specific modification, including PEGylation and dimerization, are described and demonstrated where the biological activity of CT-1 is retained. The novel protein compositions having selective modification allow for improved therapeutic properties.

Generally, according to techniques known in the art and described herein, CT-1 polypeptides may be covalently attached or conjugated to a wide variety of biocompatible polymers. In one embodiment of the invention, the pharmacokinetic parameters, including half-life, of a CT-1 polypeptide of the invention are improved by conjugation of the polypeptide to biocompatible polymers, including for example, a biocompatible polymer, such as polyethylene glycol (PEG). Accordingly, in particular embodiments, biocompatible polymers such as PEG are pharmacokinetic modulators of polypeptides of the invention. In particular embodiments, a polymer is conjugated to a CT-1 polypeptide at a desired location by site-specific modification of the protein.

In some embodiments of the invention, a CT-1 polypeptide is conjugated to at least one other CT-1 polypeptide to form an oligomer, or is conjugated to an alternative polypeptide to obtain a fusion protein. In particular embodiments, two CT-1 polypeptides are conjugated to obtain a homodimer. Multimerization of the CT-1 polypeptides of the invention may be facilitated by methods known and used in the art, and in particular embodiments of the invention, oligomerization occurs using PEG linkage.

As used herein, the term “oligomer” refers to the various “mers” of CT-1 polypeptides, such as monomer, dimer, trimer, tetramer, pentamer and hexamer. In certain embodiments, the polypeptides of the invention are dimers. The dimeric peptides may comprise CT-1 polypeptide fragments, variants, and modified CT-1 polypeptides as described elsewhere herein. Molecules may also form an oligomer if they are covalently linked to each other. For example, two distinct proteins, chemically synthesized, in vitro translated or isolated or purified from a cell or a sample, may be chemically cross-linked together via a non-amide bond to form an oligomer. Thus, two molecules that exist as separate entities and that do not form oligomers through non-covalent interaction but are joined together via covalent bonds are also considered to be a multimer. Accordingly, an oligomer or a multimer of the invention (e.g., hetero- or homo-dimer, trimer, tetramer, pentamer, hexamers, etc.) may be formed through covalent bonding, non-covalent bonding or mixtures thereof.

Thus, multimers, particularly dimers, can be formed by joining two or more monomers through intermolecular disulfide bonds or by crosslinking agents such as, for example, functionalized biocompatible polymers, e.g., polyethylene glycol, other polyethers, EDTA and other linkers known to those skilled in the art. See, for example U.S. Patent Application Publication Number 2001/0008341, which is hereby incorporated by reference in its entirety. In various embodiments, the functionalized biocompatible polymers used to form dimers may comprise, for example, 1, 2, 3, 4, 5, 10, 15, 20, 25, 30, 35, 40, 45, or 50 PEG monomer units. In particular embodiments the number of monomeric units can be from 1 to 50, 2 to 30, 3 to 20, or 4 to 12.

In other embodiments, the number of PEG monomer units is such that molecular weight of the PEG polymer itself is less than 2 kDa, less than 1.5 kDa, less than 1 kDa, or less than 500 Da. In more particular embodiments, the molecular weight of the PEG polymer is about 2 kDa, about 1.8 kDa, about 1.8 kDa, about 1.5 kDa, about 1.2 kDa, about 1.0 kDa, about 0.8 kDa, about 0.7 kDa, about 0.6 kDa, about 0.5 kDa, or about 0.2 kDa.

In certain embodiments, the individual CT-1 polypeptide monomers can be separated from the PEG chain by spacer groups. For example, spacer groups having between 0 and 10 carbons such as between 1 and 10 carbons, between 1 and 6 carbons or between 1 and 4 carbons, and optionally containing one or more amide linkages, ether linkages, ester linkages, and the like. Spacers groups between the individual CT-1 polypeptide monomers and the PEG chain can be the same or different, and can be straight-chained, branched or cyclic (for example, aliphatic or aromatic cyclic structures), and can be unsubstituted or substituted. Functional groups that can be substituents on a spacer include carbonyl groups, hydroxyl groups, halogen (F, Cl, Br and I) atoms, alkoxy groups (such as methoxy and ethoxy), nitro groups, and sulfato groups.

In additional embodiments, amino acid sequences that confer multimerization and mediate protein-protein binding via Van der Waals' forces, hydrophobic interactions, hydrogen bonding or charge-charge bonds may be included in CT-1 polypeptides of the invention. Thus, the invention provides methods of producing a multimerization polypeptide comprising multimerization domains, see, e.g., U.S. Patent Application Publication Number 2003/0138440 and International PCT Publication WO2003/062370, each of which are hereby incorporated by reference in their entirety.

Exemplary biocompatible polymers include, but are not limited to non-peptide polymers (e.g., at least 2 covalently linked non-peptide moieties) include, for example, polyethylene glycol (PEG), polypropylene glycol, copolymers of ethylene glycol and propylene glycol, polyoxyethylated polyols, polyvinyl alcohol, oligosaccharides, dextran, polyvinyl ethyl ether, biodegradable polymers, lipid polymers, chitins, hyaluronic acid and combinations thereof. Examples of suitable non-peptide polymers can be found, for example, in Duncan (2003,), inclusive of PEG, N-(2-hydroxypropyl) methacrylamide (HPMA), polyvinylpyrrolidone (PVP), and poly-ethyleneimine (PEI).

In particular embodiments, polymer molecules used in chemically modifying the CT-1 polypeptides of the invention may be selected from among water soluble polymers. The polymer selected may be water soluble so that the protein to which it is attached does not precipitate in an aqueous environment, such as a physiological environment. For therapeutic use of the end-product protein, the polymer will be pharmaceutically acceptable. The water soluble polymer may be selected from the group consisting of, for example, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols and polyvinyl alcohol.

In certain embodiments, typically following production (e.g., recombinant, synthetic) and optionally isolation of modified CT-1 polypeptides, or variants thereof, these polypeptides may be chemically modified in vitro by the covalent attachment of one or more non-peptide polymers (e.g., a biocompatible polymer). Also, modified CT-1 polypeptides may be produced synthetically from pre-modified building blocks, such as by synthesizing the CT-1 polypeptide in vitro from selected amino acids that are already covalently attached to a non-peptide polymer.

Alternatively or in combination with in vitro modification, in certain embodiments the recombinant CT-1 polypeptides may be expressed in a host cell that is designed to attach a biocompatible polymer comprising one or more non-peptide moieties to the polypeptide, such as a host cell that is capable of glycosylating the CT-1 polypeptide (e.g., N-linked glycosylation). Included are host cells that are naturally capable of glycosylating proteins (e.g., eukaryotic cells), and those that are engineered to contain suitable glycosylation machinery (see, e.g., Feldman et al., PNAS USA 102:3016-3021, 2005 for engineering E. coli to modify proteins by N-linked glycosylation). In these embodiments, the modified CT-1 polypeptide may be isolated from the host cell, and either further chemically modified (e.g., PEGylation), or used in its already modified form.

In certain embodiments, the CT-1 polypeptides of the invention may be PEGylated according to techniques in the art. Modification by PEG typically occurs in vitro. As one non-limiting example, PEG's most common form is linear or branched polyether with terminal hydroxyl groups synthesized by anionic ring opening polymerization —HO—(CH₂CH₂O)ₙ—CH₂CH₂—OH. Monomethoxy PEG (mPEG) may be particularly useful for peptide modification —CH₃—O(CH₂CH₂O)ₙ—CH₂CH₂—OH or CH₃—O(CH₂CH₂O)ₙ—H because it can be derivatized in a number of linkage moieties, such as methoxy-PEG amines, maleimides and carboxylic acid. Certain general factors may affect the performance of PEGylated polypeptides, including molecular weight and structure (e.g., generally, PEGS of >1000 Da have not demonstrated any toxicity in vivo, and PEGs of up to 40,000-50,000 Da or more have been used in clinical and approved pharmaceutical applications), number of PEG chains (e.g., two or more lower-weight chains can be added to increase the total molecular weight of the PEG complex), the specific location of PEG site of attachment to the polypeptide, the chemistry used to attach the PEG to the polypeptide, including the purity of raw materials, intermediates, and the final product.

The CT-1 polypeptides of the invention may be modified by attachment (particularly, site-specific attachment) to PEG molecules of a variety of molecular weights, including, but not limited to PEGs of about 1000, 2000, 3000, 4000, 5000, 6000, 7000, 8000, 9000, 10,000, 15,000, 20,000, 25,000, 30,000, 40,000, 50,000, 60,000, 70,000, 80,000, 90,000, 100,000 Da or more, including all integers in between. Certain preferred embodiments include PEG of a molecular weight of about 20,000 Da or 40,000 Da.

Typically, as a non-limiting example, the first step in coupling PEG (e.g., mPEG) to a polypeptide is to activate the PEG with a functional group at one or both termini. The choice of a functional group mainly depends on the available or selected reactive groups on the polypeptide (e.g., lysine, aspartic acid, cysteine, glutamic acid, serine, threonine, the N-terminal amine, the C-terminal carboxylic acid, or other specific site). Both weak and relatively strong linkages to the polypeptide can be used. For instance, strong linkages are generally consistent with the objective of increasing the circulating half-life and solution stability of the polypeptide. However, in slightly acidic conditions, succinimidyl carbonate linkages to histidine will form an imidazolecarbamate that will hydrolyze in a controlled release fashion in vivo—a process that can be fine tuned to occur in endogenous circulation over hours, days, or weeks.

PEGylation may occur at any or all of numerous nucleophilic sites in a protein, including, for example, the s-amino groups of lysines, the α-amino group at cysteines, the imidazolyl nitrogens of histidines, and the hydroxyl groups of serines, threonines, and tyrosines. Because of the numerous potential reaction sites, in certain instances a heterogeneous mixture of various different modified proteins may be produced.

In one embodiment, site-specific PEGylation may also be used to, for example, generate more homogenous mixtures. In one non-limiting example, PEG-protein conjugates can be formed by PEG aldehyde conjugation to the N-terminus of an unprotected polypeptide chain.

In another embodiment, cysteine thiols in polypeptides can be conjugated to PEG or other polymers that are activated with maleimides, vinyl sulfones, pyridyl disulfides, or other compounds specific for thiols. The conjugation of a polypeptide to such compounds can be rate- and site-controlled by monitoring and adjusting pH during the coupling.

In certain embodiments of the invention, non-peptide polymers are conjugated to a CT-1 polypeptide in a site-specific manner to provide a modified protein having CT-1 biological activity and improved pharmacokinetic properties. In the proteins of the invention, polymers such as PEG may be attached to wild-type cysteine residues (i.e., cysteine residues present in the wild-type CT-1 sequence), or to substituted cysteines (i.e., cysteine residues introduced into the wild-type sequence by addition or substitution), so as to target the biocompatible polymer comprising one or more non-peptide moieties to a desired location, such as the integrin binding site or its adjacent residues. In certain embodiments, certain of the wild-type CT-1 cysteine residues may be first substituted with another amino acid to prevent attachment of the non-peptide polymer to these wild-type cysteines, mainly to prevent the non-peptide polymer from disrupting an otherwise desirable biological activity. Carbohydrates can also be conjugated to hydradized PEG derivatives.

In certain embodiments, the CT-1 polypeptides of the invention may be glycosylated according to techniques in the art. Glycosylation may be performed in vitro or in vivo. Examples of glycosylation include S-linked, O-linked, and N-linked glycosylation (see, e.g., U.S. Application No. 2009/0053167).

O-linked glycosylation refers generally to the addition of O—N-acetyl-galactosamine (O-GalNAc) to serine or threonine residues by the enzyme UDP-N-acetyl-D-galactosamine:polypeptide N-acetylgalactosaminyltransferase, followed by other carbohydrates (such as galactose and sialic acid). O-linked glycosylation may also refer to the addition of O-fucose, O-glucose, and O—N-acetylglucosamine (O-GlcNAc). See, e.g., WO/2008/011633 for glycosylation of peptides via O-linked glycosylation sequences.

N-linked glycosylation refers generally to the attachment of oligosaccharides to asparagine residues, typically at the consensus sequence Asn-X-Ser/Thr, wherein X is any amino acid except proline. Generally, there are three major types of N-linked oligosaccharides: high-mannose oligosaccharides, complex oligosaccharides and hybrid oligosaccharides. High-mannose oligosaccharides typically refer to two N-acetylglucosamines that comprise a number of mannose residues, many of which are present in the precursor oligosaccharides before it is attached to the protein. Complex oligosaccharides typically contain almost any number of the other types of saccharides, including more than the original two N-acetylglucosamines. As one example, mature glycoproteins may contain a variety of oligomannose N-linked oligosaccharides containing between 5 and 9 mannose residues. Further removal of mannose residues leads to a “core” structure containing 3 mannose, and 2 N-acetylglucosamine residues, which may then be elongated with a variety of different monosaccharides including galactose, N-acetylglucosamine, N-acetylgalactosamine, fucose and sialic acid.

In certain embodiments, an isolated CT-1 polypeptide may be first produced by chemical synthesis or by expression in a recombinant microorganism, and then glycosylated in vitro. For instance, using peptide-N-glycosidase F, enzymatic in vitro glycosylation occurs with excess maltodextrin and ammonium salt in acetone at 50° C. (see, e.g., Lee et al., Enzyme and Microbial Technology 30:716-720, 2002).

In certain embodiments, a recombinant CT-1 polypeptide may be both expressed and glycosylated in a host cell, such as a eukaryotic cell, that is capable of naturally and selectively glycosylating the polypeptide. For N-linked glycosylation in eukaryotic cells, the precursor Glc₃Man₉GlcNAc₂ oligosaccharide (i.e., 3 glucose, 9 mannose, and 2 N-acetylglucosamine) molecule is assembled on a lipid carrier (e.g., dolichyl pyrophosphate) at the membrane of the endoplasmic reticulum, and is then transferred to select Asn residues within the sequence motif Asn-X-Ser/Thr of nascent polypeptide chains. This process is catalyzed by the oligosaccharyltransferase (OST).

In certain embodiments, a recombinant CT-1 polypeptide may be both expressed and glycosylated in a host cell, such as a bacterial cell that has been engineered to contain an operable glycosylation system. As one example, the PgIB gene of the N-linked glycosylation system of Campylobacter jejuni is sufficient to confer on E. coli the ability to transfer O-polysaccharides from a lipid carrier (e.g., undecaprenyl pyrophosphate) to an acceptor protein (see, e.g., Feldman et al., supra; and WO/2004/091499 in the intracellular formation of peptide conjugates).

Other methods of conjugating polypeptides with biocompatible polymers comprising one or more non-peptide moeities can be found in the art (see, e.g., Lele et al., Biomacromolecules 6:3380-3387, 2005 for the synthesis of uniform protein-polymer conjugates; U.S. Pat. No. 5,985,263 for histidine-linked protein-polymer conjugates and processes for their preparation; Broyer et al. J. Am. Chem. Soc. 130:1041-1047, 2008 for designed amino acid ATRP Initiators for the synthesis of biohybrid materials; U.S. Pat. No. 5,998,588 and Heredia et al., Org. Biomol. Chem. 7, 45-53, 2007 for the synthesis of protein-polymer conjugates; and Liu et al., Angew. Chem. Int. Ed. 46: 3099-3103, 2008 for in situ formation of protein-polymer conjugates via reversible addition fragmentation chain transfer polymerization).

In the polypeptides of the invention having site-specific modification, covalent attachment of the modification (e.g. PEG) to the CT-1 polypeptide preferably provides a stable conjugated complex. Commonly used reactive chemistries and conjugation groups are described in Table 1.

TABLE 1

| Functional Group On Therapeutic | Conjugation Reactive group |
|---|---|
| Carboxyl (directly to amine) | Carbodiimide |
| Amine | NHS ester |
| | Imidoester |
| | PFP ester |
| | Hydroxymethyl phosphine |
| Sulfhydryl | Maleimide |
| | Haloacetyl (Bromo- or Iodo-) |
| | Pyridyldisulfide |
| | Vinyl sulfone |
| Aldehyde (Carbonyls) | Hydrazide |
| i.e., oxidized carbohydrates | |
| Any Group (Nonselective) | Diazirine (Photo-reactive) |
| | Aryl Azide (Photo-reactive) |
| Hydroxyl (non-aqueous) | Isocyanate |

To generate CT-1 variants, one skilled in the art, for example, can change one or more of the codons of the encoding DNA sequence, e.g., according to Table 2.

TABLE 2
Amino Acid Codons

| Amino Acids | Codons | | | | | |
|---|---|---|---|---|---|---|
| Alanine | GCA | GCC | GCG | GCU | | |
| Cysteine | UGC | UGU | | | | |
| Aspartic acid | GAC | GAU | | | | |
| Glutamic acid | GAA | GAG | | | | |
| Phenylalanine | UUC | UUU | | | | |
| Glycine | GGA | GGC | GGG | GGU | | |
| Histidine | CAC | CAU | | | | |
| Isoleucine | AUA | AUC | AUU | | | |
| Lysine | AAA | AAG | | | | |
| Leucine | UUA | UUG | CUA | CUC | CUG | CUU |
| Methionine | AUG | | | | | |
| Asparagine | AAC | AAU | | | | |
| Proline | CCA | CCC | CCG | CCU | | |
| Glutamine | CAA | CAG | | | | |
| Arginine | AGA | AGG | CGA | CGC | CGG | CGU |
| Serine | AGC | AGU | UCA | UCC | UCG | UCU |
| Threonine | ACA | ACC | ACG | ACU | | |
| Valine | GUA | GUC | GUG | GUU | | |
| Tryptophan | UGG | | | | | |
| Tyrosine | UAC | UAU | | | | |

Guidance in determining which amino acid residues can be substituted, inserted, or deleted without abolishing biological or immunological activity can be found using computer programs well known in the art, such as DNASTAR™ software. If desired, amino acid substitutions can be made to change and/or remove functional groups from a polypeptide. Alternatively, amino acid changes in the protein variants disclosed herein can be conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids. See TABLE 3.

TABLE 3
Conservative Amino Acid Substitutions

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative (or non-conservative) substitutions.

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

In one embodiment, amino acids in polypeptides of the present invention that play a role in protein function can be identified by site-directed mutagenesis or alanine-scanning mutagenesis, for example. Ligand-receptor binding sites can also be determined, for example, by structural analysis such as crystallization, nuclear magnetic resonance or photoaffinity labeling.

Certain changes do not significantly affect the folding or activity of the protein. The number of amino acid substitutions a skilled artisan would make depends on many factors, including those described above. Generally speaking, the number of substitutions for any given polypeptide will not be more than 50, 40, 30, 25, 20, 15, 10, 5 or 3.

Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

In certain embodiments of the invention, there are provided fusion polypeptides, and polynucleotides encoding fusion polypeptides. Fusion polypeptide and fusion proteins refer to a polypeptide of the invention that has been covalently linked, either directly or via an amino acid linker, to one or more heterologous polypeptide sequences (fusion partners). The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

The CT-1 polypeptides of the invention may be produced by any standard method, including but not limited to recombinant DNA technology and conventional synthetic methods including, but not limited to, those set forth in J. M. Stewart and J. D. Young, Solid Phase Peptide Synthesis, 2nd ed., Pierce Chemical Co., Rockford, Ill. (1984) and J. Meienhofer, Hormonal Proteins and Peptides, Vol. 2, Academic Press, New York, (1973) for solid phase synthesis and E. Schroder and K. Lubke, The Peptides, Vol. 1, Academic Press, New York, (1965) for solution synthesis. The disclosures of the foregoing treatises are incorporated by reference herein.

In preferred embodiments, CT-1 polypeptides of the invention are produced by the inventive methods disclosed herein.

### D. Polynucleotides

The invention also provides isolated polynucleotides that encode CT-1 polypeptides of the invention, e.g., SEQ ID NO: 3-25. The CT-1 polynucleotides are suitable for clinical scale production of variant CT-1 polypeptides and for use in methods of enhancing repair and regeneration in injured and diseased tissue, including cardiac and hepatic tissue, in humans.

Nucleic acids can be synthesized using protocols known in the art as described in Caruthers et al., 1992, Methods in Enzymology 211, 3-19; Thompson et al., International PCT Publication No. WO 99/54459; Wincott et al., 1995, Nucleic Acids Res. 23, 2677-2684; Wincott et al., 1997, Methods Mol. Bio., 74, 59-68; Brennan et al., 1998, Biotechnol Bioeng., 61, 33-45; and Brennan, U.S. Pat. No. 6,001,311).

As will be understood by those skilled in the art, the polynucleotide sequences of this invention can include genomic sequences, extra-genomic and plasmid-encoded sequences and smaller engineered gene segments that express, or may be adapted to express, proteins, polypeptides, peptides, and the like. Such segments may be naturally isolated, recombinant, or modified synthetically by the hand of man.

As will be recognized by the skilled artisan, polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide of the invention or a portion thereof) or may comprise a variant, or a biological functional equivalent of such a sequence. Polynucleotide variants may contain one or more substitutions, additions, deletions and/or insertions, as described elsewhere herein, preferably such that the variant encodes a polypeptide that lacks canonical lipidation sites, but retains, and in some embodiments, has increased biological activity, such as pathway signaling activity.

Also included are polynucleotides that hybridize to polynucleotides that encode a polypeptide of the invention. To hybridize under “stringent conditions” describes hybridization protocols in which nucleotide sequences at least 60% identical to each other remain hybridized. High stringency hybridization conditions are conditions that enable a probe, primer or oligonucleotide to hybridize only to its target sequence. Stringent conditions are sequence-dependent and will differ. Moderately stringent conditions are conditions that use washing solutions and hybridization conditions that are less stringent (Sambrook, 1989) than those for high stringency, such that a polynucleotide will hybridize to the entire, fragments, derivatives or analogs of nucleic acids of the present invention. Moderate stringency conditions are described in (Ausubel et al., 1987; Kriegler, 1990). Low stringent conditions are conditions that use washing solutions and hybridization conditions that are less stringent than those for moderate stringency (Sambrook, 1989), such that a polynucleotide will hybridize to the entire, fragments, derivatives or analogs of nucleic acids of the present invention.

Conditions of low stringency, such as those for cross-species hybridizations are described in (Ausubel et al., 1987; Kriegler, 1990; Shilo and Weinberg, 1981).

In additional embodiments, the invention provides isolated polynucleotides comprising various lengths of contiguous stretches of sequence identical to or complementary to a polynucleotide encoding a polypeptide as described herein. For example, polynucleotides provided by this invention encode at least about 50, 100, 150, 175, or about 200 or more contiguous amino acid residues of a polypeptide of the invention, as well as all intermediate lengths. It will be readily understood that “intermediate lengths”, in this context, means any length between the quoted values, such as 56, 57, 58, 59, etc., 101, 102, 103, etc.; 151, 152, 153, etc.; 199, 200, 201, etc.

It will be appreciated by those of ordinary skill in the art that, as a result of the degeneracy of the genetic code, there are many nucleotide sequences that encode a polypeptide as described herein, including polynucleotides that are optimized for human and/or primate codon selection. Further, alleles of the genes comprising the polynucleotide sequences provided herein may also be used.

Polynucleotides compositions of the invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references).

Polynucleotides compositions of the invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references).

### E. Expression and Purification Systems

In various embodiments, the present invention contemplates methods of expressing and purifying CT-1 polypeptides of the invention. In general, techniques for expression and purification of polypeptides have been described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. In one embodiment, CT-1 polypeptides of the invention are prepared by a procedure including one or more of the steps of: (a) preparing a construct comprising a polynucleotide sequence that encodes a CT-1 polypeptide and that is operably linked to a regulatory element; (b) introducing the construct into a host cell; (c) culturing the host cell to express the CT-1 polypeptide; and (d) isolating CT-1 polypeptide from the host cell.

A variety of systems for expressing polynucleotide sequences are known and may be utilized to contain and express polynucleotide sequences (“expression systems”). Such expression systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems (including mammalian host cells).

As used herein, the terms “control elements” or “regulatory sequences” refer to those sequences present in an expression vector that are non-translated regions of the vector, e.g., enhancers, promoters, 5′ and 3′ untranslated regions, and interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.), pET plasmid (Novagen) and the like may be used.

In the yeast Saccharomyces cerevisiae, a number of vectors containing constitutive or inducible promoters such as alpha factor, alcohol oxidase, and PGH may be used. Also included are Pichia pastoris expression systems (see, e.g., Li et al., Nature Biotechnology. 24, 210-215, 2006; and Hamilton et al., Science, 301:1244, 2003).

In cases where plant expression vectors are used, viral promoters such as the 35 S and 19 S promoters of CaMV may be used alone or in combination with the omega leader sequence from TMV (Takamatsu, EMBO J. 6:307-311 (1987)). These constructs can be introduced into plant cells by direct DNA transformation or pathogen-mediated transfection.

An insect system may also be used to express a polypeptide of interest. Exemplary baculovirus expression systems, include, but are not limited to those that utilize SF9, SF21, and Tni cells (see, e.g., Murphy and Piwnica-Worms, Curr Protoc Protein Sci. Chapter 5:Unit5.4, 2001).

In mammalian host cells, a number of viral-based expression systems are generally available. In addition, transcription enhancers, such as the Rous sarcoma virus (RSV) enhancer, may be used to increase expression in mammalian host cells. Examples of useful mammalian host cell lines include COS-7 cells, 293 or 293 T cells, BHK cells, VERO-76 cells, HELA cells, and CHO cells, including DHFR-CHO cells. Mammalian expression systems can utilize attached cell lines, for example, in T-flasks, roller bottles, or cell factories, or suspension cultures, for example, in 1 L and 5 L spinners, 5 L, 14 L, 40 L, 100 L and 200 L stir tank bioreactors, or 20/50 L and 100/200 L WAVE bioreactors, among others known in the art.

Also included is cell-free expression of proteins. These and related embodiments typically utilize purified RNA polymerase, ribosomes, tRNA and ribonucleotides; these reagents may be produced by extraction from cells or from a cell-based expression system.

In particular embodiments, polypeptides of the invention are expressed and purified from bacteria. Exemplary bacterial expression vectors include, BLUESCRIPT (Stratagene); pIN vectors (Van Heeke & Schuster, J. Biol. Chem. 264:5503 5509 (1989)); and pGEX Vectors (Promega, Madison, Wis.) which may be used to express foreign polypeptides as fusion proteins with glutathione S-transferase (GST). Certain embodiments may employ E. coli-based expression systems.

In specific embodiments, protein expression may be controlled by a T7 RNA polymerase (e.g., pET vector series). These and related embodiments may utilize the expression host strain BL21 (DE3), a λDE3 lysogen of BL21 that supports T7-mediated expression and is deficient in Ion and ompT proteases for improved target protein stability. Also included are expression host strains carrying plasmids encoding tRNAs rarely used in E. coli, such as Rosetta™ (DE3) and Rosetta 2 (DE3) strains. Cell lysis and sample handling may also be improved using reagents such as Benzonase® nuclease and BugBuster® Protein Extraction Reagent. For cell culture, auto-inducing media can improve the efficiency of many expression system, including high-throughput expression systems. Media of this type (e.g., Overnight Express™ Autoinduction System) gradually elicit protein expression through metabolic shift without the addition of artificial inducing agents such as IPTG. Certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

The protein produced by a recombinant cell can be purified and characterized according to a variety of techniques. Exemplary systems for performing protein purification and analyzing protein purity include fast protein liquid chromatography (FPLC) (e.g., AKTA and Bio-Rad FPLC systems), high-pressure liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HyperD® ion exchange chromatography, and hydrophobic interaction columns (HIC), among others known in the art. Also included are analytical methods such as SDS-PAGE (e.g., coomassie, silver stain), immunoblot, Bradford, and ELISA, which may be utilized during any step of the production or purification process, typically to measure the purity of the protein composition.

In certain embodiments, clinical grade proteins can be isolated from E. coli inclusion bodies. In particular embodiments, the present invention contemplates methods (as described in the examples and elsewhere herein) for producing a recombinant CT-1 polypeptide that is suitable for therapeutic uses.

In one embodiment, a method for producing a recombinant CT-1 polypeptide includes one or more of the following steps: i) expression of a CT-1 polynucleotide in a host; ii) culturing the host cell to express the CT-1 polypeptide, e.g., SEQ ID NOs: 2-25, as inclusion bodies; iii) one or more steps of washing the inclusion bodies; iv) solubilizing the polypeptide; v) refolding the polypeptide; vi) purifying the polypeptide; and vii) dializing the polypeptide in a desired buffer.

In certain embodiments, CT-1 polynucleotide sequences are codon optimized for expression in a bacterial host. In related embodiments, the host is E. coli. CT-1 polynucleotides are cloned into bacterial expression vectors, e.g., pET-27b(+).

In particular embodiments, BL21 (DE3) E. coli cells are transformed with bacterial expression plasmids that encode a CT-1 polypeptide of the invention. Once cells have been transformed, fresh cultures may be prepared by diluting the culture into an autoinduction medium supplemented with the appropriate antibiotic, e.g., 25-fold dilution of transformed cells into 0.5 L Overnight Express (EMD) autoinduction medium prepared in 2×YT supplemented with 100 μg/mL kanamycin. The cultures can then be grown in incubators at a suitable temperature and for a duration sufficient to express the protein to a desired level, e.g., 37° C. for 16 hours. Cells can then be harvested by centrifugation and washed with PBS.

Thus in one embodiment, a method of preparing a CT-1 polypeptide comprises one or more steps of the following: cloning a CT-1 polynucleotide into a bacterial expression vector, transforming the bacterial expression vector into E. coli, expressing the CT-1 polypeptide at a time and temperature sufficient to produce CT-1 polypeptide in inclusion bodies, lysing the bacterial cells to obtain inclusion bodies, washing the inclusion bodies, solubilizing the inclusion bodies, refolding solubilized CT-1 polypeptides, purifying CT-1 polypeptides away from host cell proteins by ion exchange chromatography, and exchanging the pure polypeptides into neutral buffer (PBS) and filtering the pure polypeptides.

In a particular embodiment, a method of purifying a CT-1 polypeptide comprises one or more of the following steps: expressing a CT-1 polypeptide in E. coli; lysing the E. coli to obtain inclusion bodies; washing the inclusion bodies one, two, three, four, five, six or more times in one or more wash buffers; solubilizing the inclusion bodies in solubilization buffer, refolding the solubilized CT-1 polypeptide against refolding buffer; concentrating the soluble refolded polypeptide; filtering the polypeptide; performing an ion exchange chromatography, purification; and dialyzing the polypeptide into a desired buffer.

In one particular embodiment, a method of purifying a CT-1 polypeptide comprises one or more of the following steps: expressing a CT-1 polypeptide as discussed elsewhere herein; resuspending E. coli pellets in lysis buffer, e.g., 50 mM Tris-HCl, pH 8.0, 100 mM NaCl, 1 mM EDTA, 10 mM DTT, supplemented with complete protease inhibitor cocktail (Roche) and 0.1% w/v sodium deoxycholate; lysing the cells by sonication; washing the inclusion bodies one, two, three, four, five, six or more times in one or more wash buffers, e.g., wash three times in Triton wash buffer (50 mM Tris-HCl, 100 mM NaCl, 0.5% Triton X-100, 1 mM EDTA and 1 mM DTT, pH 8.0) and three times by Tris wash buffer (50 mM Tris-HCl, 1 mM EDTA and 1 mM DTT, pH 8.0); solubilizing the inclusion bodies in solubilization buffer, e.g., 100 mM sodium acetate, 2 M urea, 2 mM DTT, pH 3.5 at room temperature; concentrating insoluble protein, e.g., centrifuging at 22,000×g for 30 minutes; dializing the supernatant that contains the soluble CT-1 polypeptide against refolding buffer, e.g., 20 mM HEPES, 2 mM DTT, pH 7.5, at 4° C. for 16 hours; concentrating the soluble refolded polypeptide, e.g., centrifuging at 22,000×g for 30 minutes; filtering the polypeptide and performing ion exchange chromatography, e.g., filter with a 0.22 μm filter and loading onto a HiTrap CM FF ion exchange column (5 mL, GE Life Sciences) pre-equilibrated with Solvent A (10 mM sodium phosphate, pH 7.5); washing the column and eluting stepwise by solvent A in 0.1 M NaCl then by solvent A in 1 M NaCl; pooling the eluates and performing dialysis against a suitable buffer, e.g., PBS at 4° C. for 16 hours.

### F. Compositions and Formulations

In various embodiments, the invention contemplates, in part, novel compositions and formulations of polypeptides having at least one biological activity of CT-1, and polynucleotides encoding the same. In particular embodiments, the invention provides aqueous formulations of soluble polypeptides having CT-1 activity to promote tissue growth, protection, regeneration, maintenance and repair.

In one embodiment, the invention contemplates, in part, compositions comprising polypeptides having at least one biological activity of CT-1, or biologically active fragments thereof, e.g., SEQ ID NOs: 2-b 25, wherein the composition is suitable for therapeutic use. In particular embodiments of the invention, the composition comprises purified isolated CT-1 polypeptide and the composition is substantially free from endotoxin, has little or no aggregate formation, and the purified isolated polypeptide of the composition is soluble in a therapeutically acceptable formulation.

In one embodiment, the invention contemplates compositions comprising polypeptides or biologically active fragments thereof having at least one biological activity of CT-1 wherein the purified isolated polypeptide and composition are substantially free from endotoxin, wherein the purified isolated polypeptide has little or no aggregate formation, wherein the purified isolated polypeptide is soluble in a therapeutically acceptable formulation and wherein the composition is substantially free of mammalian proinflammatory agents.

Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipooligosaccharides (LOS) found in the outer membrane of various Gram-negative bacteria, and which represent a central pathogenic feature in the ability of these bacteria to cause disease. Small amounts of endotoxin in humans can produce fever, a lowering of the blood pressure, and activation of inflammation and coagulation, among other adverse physiological effects. Therefore, it is often desirable to remove most or all traces of endotoxin from drugs and drug product containers, because even small amounts may cause adverse effects in humans. Producing formulations that are endotoxin-free can require special equipment, expert artisans, and can be significantly more expensive than making formulations that are not endotoxin-free.

Endotoxins can be detected using routine techniques known in the art. For example, the Limulus Ameobocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). As used herein, the term “endotoxin free” refers to compositions that contain at most trace amounts (i.e., amounts having no adverse physiological effects to a subject) of endotoxin, and preferably undetectable amounts of endotoxin. In one embodiment, the term “endotoxin free” refers to a composition that is at least 95%, at least 96%, at least 97%, at least 98%, at least 99%, or 100% endotoxin free. In one embodiment, the term “endotoxin free” refers to endotoxin levels or an endotoxin profile that may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 endotoxin units (EU)/ml or EU/mg. Typically, 1 ng lipopolysaccharide (LPS) corresponds to about 1-10 EU.

To be substantially endotoxin free, endotoxin levels or endotoxin profile may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/ml.

In certain embodiments, the invention contemplates, in part, a CT-1 polypeptide or fragment thereof having at least one biological activity of CT-1, and comprising an endotoxin profile of less than about 50 EU/mg, less than about 30 EU/mg, less than about 25 EU/mg, less than about 20 EU/mg, less than about 15 EU/mg, less than about 10 EU/mg, less than about 8 EU/mg, less than about 7 EU/mg, less than about 6 EU/mg, less than about 5 EU/mg, less than about 4 EU/mg, less than about 3 EU/mg less than about 2 EU/mg, less than about 1.5 EU/mg, less than about 1.4 EU/mg, less than about 1.3 EU/mg, less than about 1.2 EU/mg, less than about 1.1 EU/mg, less than about 1.0 EU/mg, less than about 0.9 EU/mg, less than about 0.8 EU/mg, less than about 0.7 EU/mg, less than about 0.6 EU/mg, less than about 0.5 EU/mg, less than about 0.4 EU/mg, less than about 0.3 EU/mg, less than about 0.2 EU/mg, less than about 0.1 EU/mg or less endotoxin units per mg of purified CT-1 polypeptide. The endotoxin levels or profile may be assessed at room temperature (20° C.-25° C.) or at body temperature (37° C.).

Exemplary mammalian proinflammatory agents, include, but are not limited to pro-inflammatory cytokines such as IL1-alpha, IL1-beta, IL6, and TNF-alpha. Other pro-inflammatory mediators include LIF, IFN-gamma, OSM, CNTF, TGF-beta, GM-CSF, IL11, IL12, IL17, IL18, IL8 and a variety of other chemokines that chemoattract inflammatory cells. These cytokines either act as endogenous pyrogens (IL1, IL6, TNF-alpha), upregulate the synthesis of secondary mediators and pro-inflammatory cytokines by both macrophages and mesenchymal cells.

The inflammatory response can be assayed using any well known animal model system. In one non-limiting example, a composition of the invention is injected into a rodent, e.g., mouse, hamster, rat, guinea pig, and the secretion of at least one, two, three, or more pro-inflammatory cytokines is measured and compared to sham injected controls. The inflammatory response may be measured as a function of time, e.g., at 30 min., 1 hour, 2 hours, 3 hours, 4 hours, or more after the animals are injected.

The invention additionally provides a CT-1 polypeptide having the amino acid sequence set forth in any one of SEQ ID NOs: 2-25 that is prepared by the production method of the invention wherein the CT-1 polypeptide possesses improved stability as compared to the naturally occurring, wild-type, or variant CT-1 prepared by other means.

Stability can generally be defined as the propensity of the molecule to remain in its folded and active state, and can be assessed using a variety of methods known in the art, including, but not limited to, measuring the amount of protein that remains active at a given time and temperature. Compositions comprising CT-1 polypeptides produced by the methods of the invention (whether CT-1 of wild type sequence or the novel CT-1 variants described herein) having improved stability, have a higher percentage of active protein when measured at a given time point and temperature as compared to compositions comprising a CT-1 polypeptide not produced by the methods of the invention. Naturally occurring molecules are usually of limited stability as their metabolism, and often their fast metabolism, is a key characteristic of their intrinsic mechanism of action in the body.

Usually, a stable protein in its folded and native structure cannot be degraded by proteases or other mechanisms. It is due to two key off pathways from the stable state by which proteins are usually eliminated in the body. These two are unfolding and aggregation. They are usually linked. Unfolding is the pathway of reverting the folded active molecule into a less folded state. Aggregation is the result of misfolding such that the molecule irreversibly turns into a non-active state. Both unfolding and aggregation significantly increase the protein's susceptibility to proteolytic or other digestion. The present invention provides a modified folding and unfolding pathway of CT-1 such that the resulting entity is more stable than a CT-1 polypeptide that is not produced by the methods of the invention.

In particular embodiments, the invention provides a CT-1 composition having increased stability against insoluble protein aggregate formation compared to a CT-1 polypeptide that is not produced by the inventive methods disclosed herein. “Protein aggregate” or “protein aggregation” is used herein to refer to protein, i.e., CT-1 polypeptide, that is no longer in solution. While protein aggregate can refer to agglomeration or oligomerization of two or more individual protein molecules, it is not limited to such a definition. Protein aggregates, as used in the art, can be soluble or insoluble; however, for the purposes of particular embodiments of the invention, protein aggregates are usually considered to be insoluble, unless otherwise specifically noted. Insoluble aggregates whose formation should be prevented in the process according to the invention are essentially understood as protein aggregates having a size of at least 1 μm but can also be in the range above 10 μm. The particles can be determined by suitable particle counting methods using commercial particle counting instruments such as, for example, the particle counting instrument AccuSizer 700 from PSS (Particle Sizing Systems, USA) or a Pacific Scientific HIAC Royco liquid particle counting system, model 9703, equipped with a LD400 laser counter. According to the USP (US-Pharmacopoeia) a maximum of 6000 particles in the range above 10 μm and a maximum of 600 particles in the range above 25 μm are allowed per injected dose of a pharmaceutical preparation. This can be achieved according to the invention to provide for therapeutic compositions of CT-1 polypeptides.

In a particular embodiments, the CT-1 composition of the invention has increased stability against aggregate formation induced by one or more freeze/thaw cycles, agitation stress, or one or more outside physical or chemical stresses including non-limiting examples of heat stress, chemical stress (e.g., pH, low/high salt, and the like), fluid stress (e.g., compression stresses, such as those caused by fluid movement through constricted openings), and the like, comprising a CT-1 polypeptide relative to a composition comprising a CT-1 polypeptide that is not produced by the inventive methods disclosed herein. As used herein “agitation stress” is taken to mean any physical movement applied to the CT-1 composition either passively or actively. Non-limiting examples of agitation stresses, include bumping, dropping, shaking, swirling, vortexing, decanting, injecting, withdrawing (as into a syringe from a containing or vessel), and the like. The CT-1 polypeptide of the invention is particularly stabilized with respect to the forces of shipping and transportation.

One measure of stability that may be employed in the context of the present invention is to compare aggregation of a polypeptide of the invention over time with that of CT-1 not prepared by the methods of the invention. For example, CT-1 polypeptide produced by the method of the invention, either wild type sequence or a novel variant of the invention, and commercially available CT-1 (ie., not prepared by the methods of the invention), can be stored at a range of temperatures (for example from 5° C. to 45° C.) and then analyzed for breakdown products and aggregated material using routine methods known in the art. A stable protein better remains in folded state and is less prone to breakdown and aggregation than a CT-1 polypeptide not produced by the methods of the invention.

CT-1 polypeptides produced by the method of the invention (whether CT-1 of wild-type sequence or the novel CT-1 variants described herein) having improved stability, retain a higher percentage of residual activity when measured at a given time point (e.g., at 2 hours, at 4 hours, at 6 hours, at 7 hours, at 8 hours, at 9 hours, at 10 hours, at 11 hours, at 12 hours, at 15 hours, at 18 hours, at 21 hours, at 24 hours) and a given temperature compared to wild-type CT-1 protein or a CT-1 polypeptide not produced by the methods of the invention. The percentage of residual (i.e., folded, active) protein may be measured by routine biochemical techniques such as HPLC, SDS PAGE or by activity assays such as binding assays or eliciting a response from cells.

CT-1 polypeptide produced by the method of the invention and having improved stability generally provides for a higher expression and higher yield in downstream processing which results in improved cost of goods (COG). Further, polypeptides produced by the method of the invention and having improved stability have an improved shelf life relative to a CT-1 polypeptide not produced by the methods of the invention. Longer shelf life is beneficial as it also influences the cost of goods.

CT-1 polypeptide produced by the method of the invention and having improved stability may have increased efficacy in the body, resulting from a longer half life. Further, CT-1 polypeptides with improved stability may be more amenable to routes of administration such as subcutaneous administration, because of reduced aggregation, which not only increases efficacy but also reduces the risk of neutralizing or binding antibodies being elicited.

In particular embodiments, the present invention contemplates compositions comprising polypeptides having at least one biological activity of CT-1, wherein the polypeptides are stable for at least 6 hours, at least 7 hours, at least 8 hours, at least 9 hours, at least 10 hours, at least 11 hours, at least 12 hours, at least 15 hours, at least 18 hours, at least 21 hours, at least 24 hours, at least 48 hours, or more, at about 37° C. compared to a non-modified polypeptide (e.g., a naturally occurring polypeptide), or compared to CT-1 (e.g., wild-type or variant) polypeptide that is not formulated according to the methods of the present invention.

The term “solubility” refers to the property of an agent provided herein to dissolve in a liquid solvent and form a homogeneous solution. Solubility is typically expressed as a concentration, either by mass of solute per unit volume of solvent (g of solute per kg of solvent, g per dL (100 mL), mg/mL, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH. In certain embodiments, solubility is measured in water or a physiological buffer such as PBS. In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent such as a CT-1 polypeptide of the invention has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 mg/mL at room temperature (20° C.−25° C.) or at 37° C.

Protein characteristics including purity, solubility and degree of aggregation can be assessed using protein-based analytical assays and methods. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroisim (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts.

Examples of methods for assessing protein characteristics such as size include analytical ultracentrifugation and size exclusion HPLC (SEC-HPLC, or alternatively, HPLC-SEC), and exemplary methods for measuring charge include ion-exchange chromatography and isolectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

Certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15: 1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade proteins such as protein fragments and antibodies can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005).

In certain embodiments, compositions of polypeptides and biologically active fragments having at least one biological activity of CT-1 have a purity of at least about 90%, with respect to the polypeptide and as measured according to routine techniques in the art. In certain embodiments, such as diagnostic compositions or certain therapeutic compositions, the polypeptide compositions of the invention have a purity of at least about 95%. In specific embodiments, such as therapeutic or pharmaceutical compositions, the polypeptide compositions of the invention have a purity of at least about 97% or 98% or 99%. In other embodiments, such as when being used as reference or research reagents, polypeptides of the invention can be of lesser purity, and may have a purity of at least about 70%, 75%, 80%, or 85%. Purity can be measured overall or in relation to selected components, such as other proteins, e.g., purity on a protein basis.

Protein solubility assays are also included. Such assays can be utilized, for example, to determine optimal growth and purification conditions for recombinant production, to optimize the choice of buffer(s), and to optimize the choice of CT-1 polypeptides or biologically active fragments. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. CT-1 polypeptides or biologically active fragments with increased solubility (or reduced aggregation) compared to CT-1 polypeptides not produced by the methods of the invention can be identified or selected for according to routine techniques in the art, including simple in vivo assays for protein solubility (see, e.g., Maxwell et al., Protein Sci. 8:1908-11, 1999).

In particular embodiments, a human therapeutic composition is provided, comprising a modified polypeptide of the invention or fragment thereof as described elsewhere herein and a pharmokinetic (PK) modulator. As used herein, the term “pharmacokinetic modulator” generally refers to a polypeptide modification that increases the pharmacokinetic parameters of the polypeptide, including, without limitation, half-life, solubility, stability, activity compared to a CT-1 polypeptide that lacks the PK modulator. In one embodiment, the PK modulator comprises a biocompatible polymer conjugated to the polypeptide, including for example, polyethylene glycol (PEG).

The compositions of the invention may comprise one or more polypeptides, polynucleotides, vectors comprising same, etc., as described herein, and one or more pharmaceutically-acceptable salts or carriers and/or physiologically-acceptable solutions for administration to a cell or an animal, either alone, or in combination with one or more other modalities of therapy. It will also be understood that, if desired, the compositions of the invention may be administered in combination with other agents as well, such as, e.g., other proteins, polypeptides, small molecules or various pharmaceutically-active agents. There is virtually no limit to other components that may also be included in the compositions, provided that the additional agents do not adversely affect the therapeutic potential of the CT-1 composition, such as the ability of the composition to promote tissue growth, regeneration, maintenance, protection and repair.

The CT-1 compositions of the invention may be made up in a solid form (including granules, powders or suppositories) or in a liquid form (e.g., solutions, suspensions, or emulsions). The compositions of the invention may be applied in a variety of solutions. Suitable solutions for use in accordance with the invention are sterile, dissolve sufficient amounts of the peptide, and are not harmful for the proposed application. In this regard, the compositions of the present invention are very stable but are hydrolyzed by strong acids and bases. The compositions of the present invention are soluble in organic solvents and in aqueous solutions at about pH 5-9, about pH 6-8, about pH 6.5-8, about pH 7-8, about pH 7.4-8, or any intervening pH. In certain embodiments, the compositions of the invention are formulated at a pH of about 5.5, about 6, about 6.5, about 7, about 7.4, or about pH 8 or any intervening pH.

In particular embodiments, the compositions of the present invention have low ionic strength, including but not limited to about 10 mM, about 20 mM, about 30 mM, about 40 mM, about 50 mM, about 60 mM, about 70 mM, about 80, mM, about 90 mM, or about 100 mM or any intervening ionic strength.

The CT-1 compositions may be subjected to conventional pharmaceutical operations such as sterilization and/or may contain conventional adjuvants, such as preservatives, stabilizers, wetting agents, emulsifiers, and buffers.

For administration, the CT-1 active agents are ordinarily combined with one or more adjuvants appropriate for the indicated route of administration. The CT-1 compositions may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

Formulations suitable for topical administration include liquid or semi-liquid preparations suitable for penetration through the skin (e.g., liniments, lotions, ointments, creams, or pastes) and drops suitable for administration to the eye, ear, or nose.

Pharmaceutically-acceptable salts include the acid addition salts (formed with the free amino groups of the protein) and those formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

In various embodiments, the invention provides compositions comprising CT-1 polypeptides at a concentration of about 0.1 mg/mL to about 50 mg/mL, about 0.5 mg/mL to about 50 mg/mL, about 1 mg/mL to about 50 mg/mL, about 1 mg/mL to about 25 mg/mL, about 1 mg/mL to about 15 mg/mL, 1 mg/mL to about 10 mg/mL, 1 mg/mL to about 5 mg/mL, about 2 mg/mL to about 8 mg/mL, about 2 mg/mL to about 5 mg/mL, about 3 mg/mL to about 5 mg/mL, about 4 mg/mL to about 5 mg/mL, about 4 mg/mL, about 5 mg/mL to about 15 mg/mL, about 5 mg/mL to about 10 mg/mL or any intervening concentration of the polypeptide. In some embodiments, the invention comprises compositions comprising polypeptides having at least one biological activity of CT-1 at a concentration of more than about 1 mg/mL, 2 mg/mL, 3 mg/mL, 4 mg/mL, or 5 mg/mL.

In certain embodiments, the invention provides compositions comprising CT-1 polypeptides at a concentration of about 0.1 mg/mL, about 0.5 mg/mL, about 1 mg/mL, about 5 mg/mL, about 10 mg/mL, about 15 mg/mL, about 25 mg/mL, or about 50 mg/mL or any intervening concentration of the polypeptide.

In addition, the invention contemplates compositions comprising CT-1 polypeptides prepared by the methods of the invention, wherein the aggregation profile contains about 1% to about 10%, about 2% to about 9%, about 3% to about 8%, about 4% to about 7%, or about 5% to about 6% of the polypeptides as aggregates. In certain embodiments, the present invention contemplates compositions comprising CT-1 polypeptides wherein the aggregation profile contains less than about 1%, less than about 2%, less than about 3%, less than about 4%, less than about 5%, less than about 6%, less than about 7%, less than about 8%, less than about 9%, or less than about 10% of the polypeptides as aggregates.

In certain embodiments, compositions of polypeptides and biologically active fragments having at least one biological activity of CT-1 have a purity of at least about 90%, with respect to the polypeptide and as measured according to routine techniques in the art. In certain embodiments, such as diagnostic compositions or certain therapeutic compositions, the polypeptide compositions of the invention have a purity of at least about 95%. In specific embodiments, such as therapeutic or pharmaceutical compositions, the polypeptide compositions of the invention have a purity of at least about 97% or 98% or 99%. In other embodiments, such as when being used as reference or research reagents, polypeptides of the invention can be of lesser purity, and may have a purity of at least about 70%, 75%, 80%, or 85%. Purity can be measured overall or in relation to selected components, such as other proteins, e.g., purity on a protein basis.

In one embodiment, the invention contemplates compositions comprising CT-1 polypeptides wherein the half-life of the polypeptides are increased at least 1.2 fold, at least 1.5 fold, at least 2 fold, at least 2.5 fold, at least 3 fold, at least 3.5 fold, at least 4 fold, at least 4.5 fold, or at least 5 fold compared to a non-modified polypeptide (e.g., a naturally occurring CT-1 polypeptide).

In particular embodiments, the invention contemplates compositions comprising CT-1 polypeptides, wherein the polypeptides are stable for at least 6 hours, at least 7 hours, at least 8 hours, at least 9 hours, at least 10 hours, at least 11 hours, at least 12 hours, at least 15 hours, at least 18 hours, at least 21 hours, at least 24 hours, at least 48 hours, or more at about 37° C. compared to a non-modified polypeptide (e.g., a naturally occurring CT-1 polypeptide).

The invention further contemplates, in particular embodiments, compositions comprising CT-1 polypeptides that are at least 75%, at least 80%, at least 85%, at least 90%, at least 95%, at least 96%, at least 97%, at least 98%, at least 99%, or 100% percent pure by weight.

### G. Methods of Delivery

The compositions of CT-1 variant polypeptides and polynucleotides encoding CT-1 polypeptides can be administered (as proteins/polypeptides, or in the context of expression vectors for gene therapy) directly to the subject or delivered ex vivo, to cells derived from the subject (e.g., as in ex vivo gene therapy). Direct in vivo delivery of the compositions may be accomplished by any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intraarterial, intramuscular, intracardiac, intrasternal, intratendinous, intraspinal, intracranial, intrathoracic, infusion techniques or intraperitoneally. Methods of administering compositions of the invention will generally be accomplished by parenteral injection, e.g., subcutaneously, intraperitoneally, intravenously myocardial, intratumoral, peritumoral, or to the interstitial space of a tissue.

In certain embodiments, the compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The compositions of the invention may also be administered by direct injection into a tissue, such as a muscle. In some embodiments of the invention, a composition of the invention is administered by directly injecting the composition into tissue to prevent a loss of tissue in the injected tissue or to promote regeneration or repair of the injected tissue.

Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, direct microinjection of the DNA into nuclei, and viral-mediated, such as adenovirus (and adeno-associated virus) or alphavirus, all well known in the art.

In certain embodiments, it will be preferred to deliver one or more modified CT-1 compositions using a viral vector or other in vivo polynucleotide delivery technique. In a preferred embodiment, the viral vector is a non-integrating vector or a transposon-based vector. This may be achieved using any of a variety of well-known approaches, such as vectors including adenovirus, retrovirus, lentivirus, adeno-associated virus vectors (AAV), or the use of other viral vectors as expression constructs (including without limitation vaccinia virus, polioviruses and herpes viruses).

Non-viral methods may also be employed for administering the polynucleotides of the invention. In one embodiment, a polynucleotide may be administered directly to a cell via microinjection or a tissue via injection, such as by using techniques described in Dubensky et al., (1984) or Benvenisty & Reshef (1986). It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). In another embodiment, polynucleotides are administered to cells via electroporation.

### H. Methods of Treatment

Therapeutic applications of the CT-1 polypeptides and polynucleotides of the invention typically pertain to situations where there is a need to replace lost or damaged tissue, for example, after chemotherapy or radiation therapy, after muscle injury, or in the treatment or management of diseases and disorders. For example, the methods can be used with skeletal muscle stem cells in the treatment, management or prevention of degenerative muscle disorders, muscular dystrophy, neuromuscular degenerative diseases, HIV infection and the like; with neural stem cells in the treatment, management or prevention of neurodegenerative disorders, such as Parkinson's disease and Alzheimer's disease, and with cardiac muscle cells in the treatment, management or prevention of degenerative or ischemic cardiac disease, acute myocardial infarction, artherosclerosis, hypertension, restenosis, angina pectoris, rheumatic heart disease, congenital cardiovascular defects, arterial inflammation and other disease of the arteries, arterioles and capillaries, in the regeneration of valves, conductive tissue or vessel smooth muscle, and in the prevention of further disease in subjects undergoing coronary artery bypass graft. Other diseases or disorders that may be treated or prevented using the methods of the present invention may include, but are not limited to degenerative liver diseases, including cirrhosis and hepatitis and diabetes.

In certain embodiments, a use of a composition as described herein for the manufacture of a medicament for promoting tissue growth, protection, maintenance, repair, protection or regeneration in a subject in need thereof is provided. In particular embodiments, a composition as described herein is provided for use in the manufacture of a medicament for promoting tissue growth, protection, maintenance, repair, or regeneration of tissue in a subject in need thereof is provided. The term “protection” with respect to tissue, as used herein, means protection of tissue from damage or injury, including for example, and without limitation, tissue damage caused by hypoxia and/or ischemic.

The composition may be administered in an effective amount, such as a therapeutically effective amount. For in vivo treatment of human and non-human subjects, the subject is usually administered a composition comprising an effective amount of one or more modified CT-1 polypeptides of the present invention. An “effective amount” refers to an amount effective, at dosages and for periods of time necessary, to achieve the desired therapeutic or prophylactic result.

A “therapeutically effective amount” of a CT-1 polypeptide of the invention, or a composition comprising the same, may vary according to factors such as the disease state, age, sex, and weight of the individual, and the ability of a CT-1 polypeptide to elicit a desired response in the individual. A therapeutically effective amount is also one in which any toxic or detrimental effects of a CT-1 polypeptide are outweighed by the therapeutically beneficial effects. The term “therapeutically effective amount” refers to an amount of a CT-1 polypeptide or composition comprising the same that is effective to “treat” a disease or disorder in a mammal (e.g., a patient).

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount is less than the therapeutically effective amount.

In particular embodiments, compositions comprising one or more modified CT-1 polypeptides and/or polynucleotides are administered in vivo to a subject in need thereof. As used herein, the term “subject” includes, but is not limited to, a mammal, including, e.g., a human, non-human primate (e.g., baboon, orangutan, monkey), mouse, pig, cow, goat, dog, cat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate. In preferred embodiments, the subject is human. Subjects in need of treatment for a disease or condition include subjects exhibiting symptoms of such disease or condition, such as those having a disease or condition, as well as those at risk of having a disease or condition.

The dosage regimen and treatment regime will vary depending on the disease being treated, based on a variety of factors, including the type of injury, the age, weight, sex, medical condition of the individual, the severity of the condition, the route of administration, and the particular compound employed. Thus, the dosage regimen and treatment regimes can vary, but can be determined routinely by a physician using standard methods. Dosage levels of the order of between 0.1 ng/kg and 10 mg/kg body weight of the active agents per body weight are useful for all methods of use disclosed herein.

Therapy may be administered for 1 to 6 times per day at dosages determined based on the disease or condition being treated, age, weight, route of administration, and other factors. In all of these embodiments, the compositions of the invention can be administered either prior to, simultaneously with, or subsequent to a planned medical procedure, onset of disease or injury. In some instances, the CT-1 compositions may be administered to a patient for up to 30 days prior to an event, such as a planned medical procedure, and for up to 60 days post-surgery, or occurrence of other injury or onset of disease. In a particular embodiment of the invention, a CT-1 composition of the invention is administered to a patient up to one week before a planned medical procedure. In another particular embodiment, a CT-1 composition of the invention is administered to a patient within one week of the occurrence of a medical procedure, occurrence of injury or onset of disease. Treatment may continue with subsequent administration of a CT-1 composition of the invention. In a particular embodiment of the invention, a subject undergoes repeated cycles of treatment according to the method of this invention.

In another particular embodiment of the invention, the CT-1 composition is administered topically. Suitable topical doses and active ingredient concentration in the formulation are as described for subcutaneous administration.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Methods

### Molecular Biology

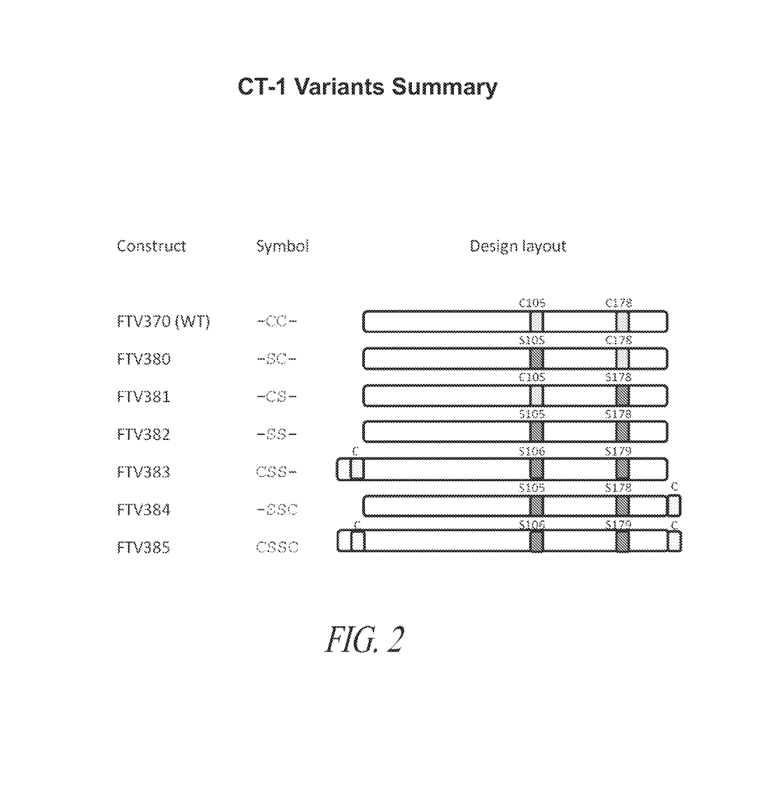

The wild-type hCT-1 DNA sequence was optimized for bacterial expression and synthesized. The gene was inserted in the pET-27b(+) vector between Nde I and BamH I sites. The C105S and C178S mutants (amino acid position with respect to the naturally occurring CT-1 polypeptide) were prepared using QuikChange site-directed mutagenesis method. The C105S mutant was made from the WT using forward primer 5′-GCTGCTGGATGCAGTTAGCCGTCGTCAGGCAGA-3′ and reverse primer 5′-TCTGCCTGACGACGGCTAACTGCATCCAGCAGC-3′. The C178S mutant and C105S/C178S double mutant was made from the WT and C105S mutant, respectively, using forward primer 5′-AGTTCTGGGTCTGCGTGTTAGCGGTCTGTATCGTGAATG-3′ and reverse primer 5′-CATTCACGATACAGACCGCTAACACGCAGACCCAGAACT-3′. N-terminal cysteine insertion between the N-terminal methionine and adjacent serine was made by PCR amplification of the C105S/C178S double mutant using forward primer 5′-GAGATATACATATGTGCAGCCGTCGTGAAGGTAG-3′ and reverse primer 5′-GCGGCCGCGGATCCTTATGC-3′. Likewise, C-terminal cysteine insertion adjacent and 5′ to the stop codon was made by PCR amplification of the C105S/C178S double mutant using forward primer 5′-GATATACATATGAGCCGTCG-3′ and reverse primer 5′-GCGGCCGCGGATCCTTAGCATGCGCTACCACCAGGCAG-3′. The PCR products were then inserted back into pET-27b(+) vector between the Nde I and BamH I sites. Thus, eight bacterial expression vectors were generated, a schematic of which can be seen in FIG. 2. Plasmid vectors were prepared for cellular transformation using Qiagen plasmid purification kits and protocols.

### Protein Production

BL21 (DE3) E. coli cells were transformed with plasmids (as listed above) encoding either human CT-1 wild-type or CT-1 polypeptides of the invention. A fresh starter culture prepared from the transformed cells was diluted by 25-fold into 0.5 L Overnight Express (EMD) autoinduction medium prepared in 2×YT supplemented with 100 μg/mL kanamycin. The culture was grown in a shaking incubator at 37° C. for 16 hours. Cells were harvested by centrifugation and washed with PBS.

### Protein Purification

E. coli cell pellets from the expression process were resuspended in lysis buffer (50 mM Tris-HCl, pH 8.0, 100 mM NaCl, 1 mM EDTA, 10 mM DTT) supplemented with complete protease inhibitor cocktail (Roche) and 0.1% w/v sodium deoxycholate. Cells were lysed by sonication. The inclusion bodies contained in the residual pellet were washed three times with Triton wash buffer (50 mM Tris-HCl, 100 mM NaCl, 0.5% Triton X-100, 1 mM EDTA and 1 mM DTT, pH 8.0) followed by three times by Tris wash buffer (50 mM Tris-HCl, 1 mM EDTA and 1 mM DTT, pH 8.0). Washed inclusion bodies were solubilized in Solubilization buffer (100 mM sodium acetate, 2 M urea, 2 mM DTT, pH 3.5) at room temperature and centrifuged at 22,000×g for 30 minutes. The supernatant was dialyzed against refolding buffer (20 mM HEPES, 2 mM DTT, pH 7.5) at 4° C. for 16 hours. The refolded protein was centrifuged at 22,000×g for 30 minutes, filtered through a 0.22 μm filter and loaded onto a HiTrap CM FF ion exchange column (5 mL, GE Life Sciences) pre-equilibrated with solvent A (10 mM sodium phosphate, pH 7.5). The column was washed with 5 column volumes of solvent A, and the bound proteins were eluted stepwise by solvent A in 0.1 M NaCl then by solvent A in 1 M NaCl. The 0.1 M NaCl eluate was pooled and dialyzed against PBS at 4° C. for 16 hours. All proteins described in the present application were purified in this way.

### Protein Modifications

Proteins were modified by linear or branched PEGylation or dimerization as indicated. For modified CT-1 polypeptides, PEGylation with linear PEG molecules, MM(PEG)n reagents (Pierce/Thermo scientific) were used where the abbreviation is for a set of compounds having polyethylene glycol (PEG) spacers with methyl (—CH3) and sulfhydryl-reactive maleimide groups at opposite ends. The unbranched, hydrophilic, discrete-length molecules have the form Methyl-PEGn-Maleimide, where the subscript “n” denotes the number of ethylene glycol units. For branched PEG, TMM(PEG)12 (Pierce/Thermo scientific) was used. “TMM(PEG)12” denotes a branched trimethyl (TM) and maleimide (M) derivative of polyethylene glycol (PEG) for efficient and specific modification of sulfhydryl groups. Each methyl-terminated PEG (mPEG) branch contains 12 ethylene glycol units. The three branches are attached to a 4-unit PEG stem that contains a sulfhydryl-reactive maleimide group at the distal end. Large PEG molecules of 30 KDa single chain and 40 KDa branched chain were obtained from NOF America Corp. For crosslinking modified CT-1 polypeptides to form functional dimers, BM(PEG)ₙ (Pierce/Thermo scientific) reagents were used. These are homobifunctional, sulfhydryl-reactive crosslinkers that contain the maleimide group at either end of a PEG2 or PEG3 spacer. In all cases the maleimide group was spontaneously reactive with the thiol-containing proteins at neutral pH. PEG and linker products were incubated with the proteins at a 10× molar excess. Proteins were subsequently analyzed for efficiency of modification and specific activity. Proteins were purified away from unreactive PEG and/or protein as necessary.

### CT-1 Polypeptide Analytical Assessment

Coomassie-stained SDS-PAGE densitometry analysis was performed using BioRad gel-doc imaging station and software. High Performance Liquid Chromatography Size exclusion chromatography (HPLC-SEC) was performed on Agilent systems HPLC. Peak area assessments were used to evaluate homogeneity and purity of the therapeutic product.

### CT-1 Polypeptide Stability Assay

For commercial human CT-1 protein: ProSpec 500 μg/mL stock protein in acetate pH4 was diluted to 2.5 μg/mL for a total volume of 500 μL at the beginning of the experiment. Over the course of 7 days, samples were taken out of the tubes, one incubating at 37° C. and another at 4° C. 37.5 ng of protein (calculated at the beginning of the experiment), was loaded onto the gel for Western. The antibody used to detect hCT-1 was R & D Systems MAB260, followed by LI-COR secondary antibody 800 CW goat anti-mouse IgG, 926-32210. For CT-1 polypeptide produced using the methods described herein, 50 μL aliquots of hCT-1 protein at 1 mg/mL were snap frozen in liquid nitrogen and stored at −80° C. At selected time points over the course of 14 days individual aliquots were thawed and incubated at 37° C. or 4° C. At the end of 14 days, 1 μg of each aliquot (protein concentration calculated at the beginning of the experiment) was loaded onto the gel for Coomassie analysis. Also, over the course of 14 days, one 50 μL aliquot of hCT-1 protein was thawed and re-frozen at −20° C. every other day, for a total of 8 freeze-thaw cycles.

### Activity Assessments and Western Blots

C2C12 myoblasts were cultured in DMEM with 10% FBS and 1× non-essential amino acids. Eighteen hours prior to protein treatment, 1.5×10⁵ cells were seeded in each well of a 12-well plate. Immediately prior to treatment, serial dilutions of human CT-1 variants were prepared in cell culture medium. Medium was aspirated from cells and was replaced with medium containing hCT-1 protein or PBS as a negative control. After 20 minutes incubation at 37° C., cells were washed once with PBS, and lysed in 100 μL cell lysis buffer [1 mL 10× cell lysis buffer (Cell Signaling Technology, 9803S)+1 tablet Complete Mini EDTA-free protease inhibitor cocktail (Roche, 04693159001)+50 μL HALT phosphatase inhibitor cocktail (Thermo, 78420)+distilled water to 10 mL]. Cell lysates were scraped on ice and transferred into Eppendorf tubes, then centrifuged at 13,200 rpm for 10 minutes at 4° C. NuPAGE LDS Sample Buffer and Sample Reducing Agent (Invitrogen) were added to lysates. Samples were run on NuPAGE gels (Invitrogen) and transferred to nitrocellulose membranes using the Invitrogen iBlot system. Blots were blocked for 1 hour at room temperature in Odyssey blocking buffer (LI-COR, 927-40000). Primary antibodies were diluted in Odyssey blocking buffer as follows: 1:1000 anti-phospho-STAT3 (Tyr705) (Cell Signaling Technology, 9138), 1:2000 anti-STAT3 (Cell Signaling Technology, 9132), 1:4000 anti-actin (Santa Cruz Biotechnology, sc-1616). After 1 hour incubation at room temperature in primary antibody, blots were washed several times in PBS+0.1% Tween-20 for 45 minutes. The appropriate LI-COR IRDye secondary antibodies were diluted 1:10,000 in Odyssey blocking buffer (800 CW goat anti-mouse IgG, 926-32210; 680 goat anti-rabbit IgG, 926-32221; 680 donkey anti-goat IgG, 926-32224). Blots were incubated with secondary antibody for 45 minutes at room temperature, washed several times in PBS+0.1% Tween-20 for 30 minutes, then quickly washed in PBS. Blots were imaged on an Odyssey scanner (LI-COR), and band intensities were quantified using the Odyssey software. Phospho-STAT3 signal was normalized to either total STAT3 or actin signal for each sample. EC₅₀ calculations were performed using GraphPad Prism software. Westerns of CT-1 pre and post PEGylation were carried out using the same protocol but with the anti-CT-1 primary antibody MAB260 (R&D Systems).

### Example 2

### Design of Novel Modified CT-1 Polypeptides

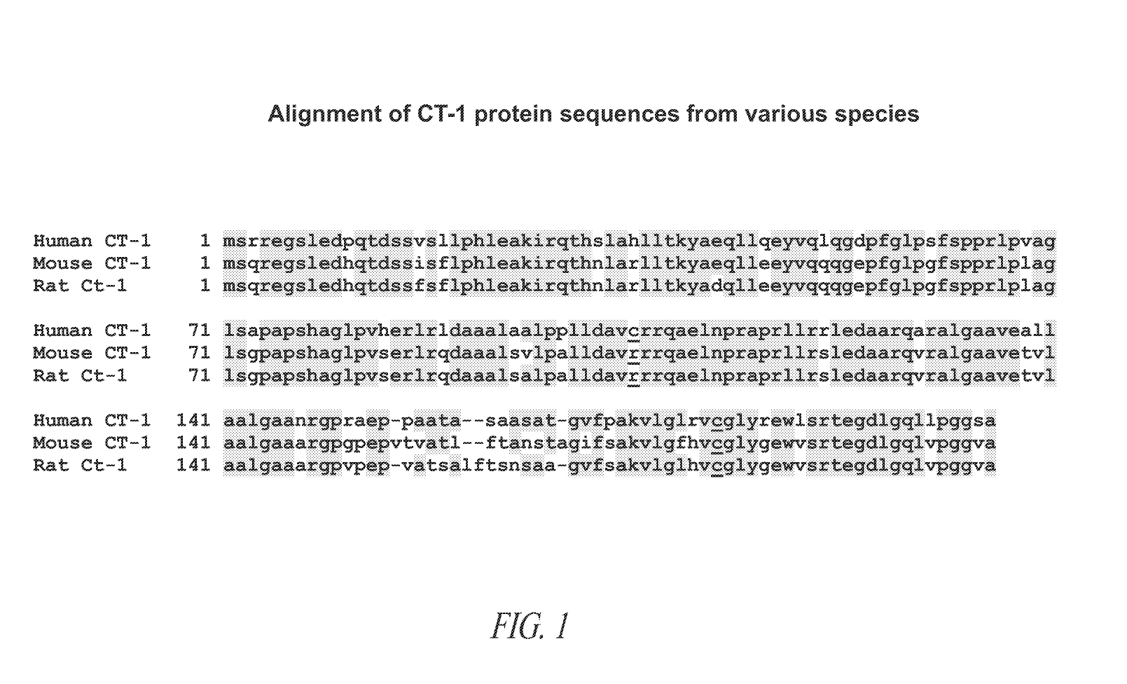

CT-1 is a small (21.5 KDa) secreted cytokine of the IL6 family with potential therapeutic applications in several areas including ischemic disease and regenerative medicine. The low molecular weight of this protein results in a short systemic half-life and bioavailability. Modifications to increase molecular weight such as PEGylation and multimerization would decrease the proteins clearance rate and absorption from a subcutaneous or intramuscular compartments thus extending the systemic half-life and required treatment interval. With such a small signaling protein there is the possibility that random or multiple sites of modification may result in a significant reduction of biological activity and/or stability. In preliminary modification experiments human CT-1 lost both biological activity and stability when primary amines within the protein were modified with reactive groups such as dimethyls linked to reactive N-hydroxysuccinimide (NHS) ester groups. Therefore site-specific modifications, targeting residues or areas permissible to modification were required. Several different chemistries are available for conjugating proteins to each other or to polymers such as PEG, as discussed elsewhere herein and as known in the art. Maleimide chemistry is commonly used to conjugate modifications to sulfydryls such as those within the thiol groups of cysteine residues. Analysis of the human CT-1 amino acid sequence showed that there were two cysteine residues, one at position 105 and a second at position 178 suggestive of an intramolecular disulfide bond. However, analysis of amino acid sequence alignments across several species (FIG. 1) indicated that the cysteine at position 178 in the human sequence was conserved whereas that at position 105 in the human sequence was not conserved between species. Further, structural analysis of the CT-1 protein suggested that the two cysteines were too distant from each other to effectively form a covalent disulfide bond. Removal of one or other of the cysteines by deletion or mutagenesis to another amino acid resulted in biological activity and stability. This resulted in a protein with a single remaining thiol group for a site-specific modification. Further, removal or replacement of both natural cysteines allowed for the addition of cysteines at other sites within the polypeptide. FIG. 2 shows a schematic of the exemplary modified CT-1 polypeptide designs along with the identifier construct name, symbol, and SEQ ID NO.

### Example 3

### Production of Wild-Type and Novel CT-1 Variants

Method of Producing Therapeutic CT-1

A scalable method of production for human CT-1 for therapeutic use has not been described. Such a method should be applicable to GLP/cGMP manufacturing and result in pure, stable, carrier-free protein that has relevant biological activity. The final formulation of the therapeutic CT-1 should ideally be applicable to direct human administration without the requirement for excessive dilution or re-formulation. The present and subsequent examples describe a suitable method for production of therapeutically useful polypeptides having at least one biological activity of CT-1.

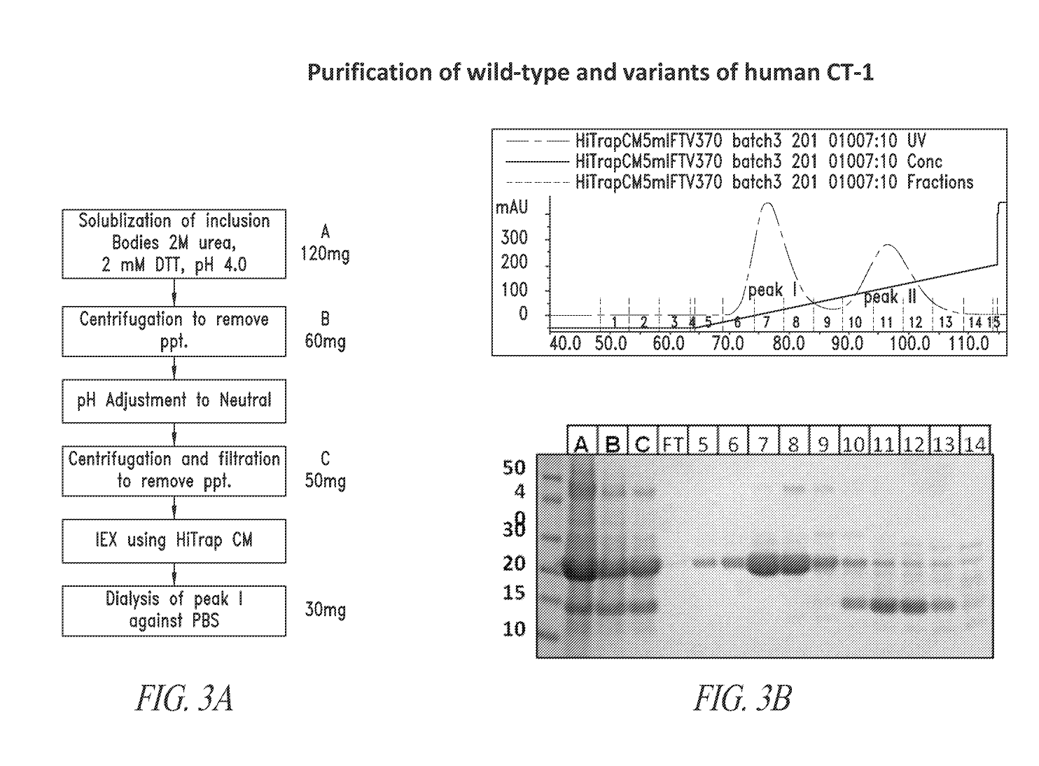

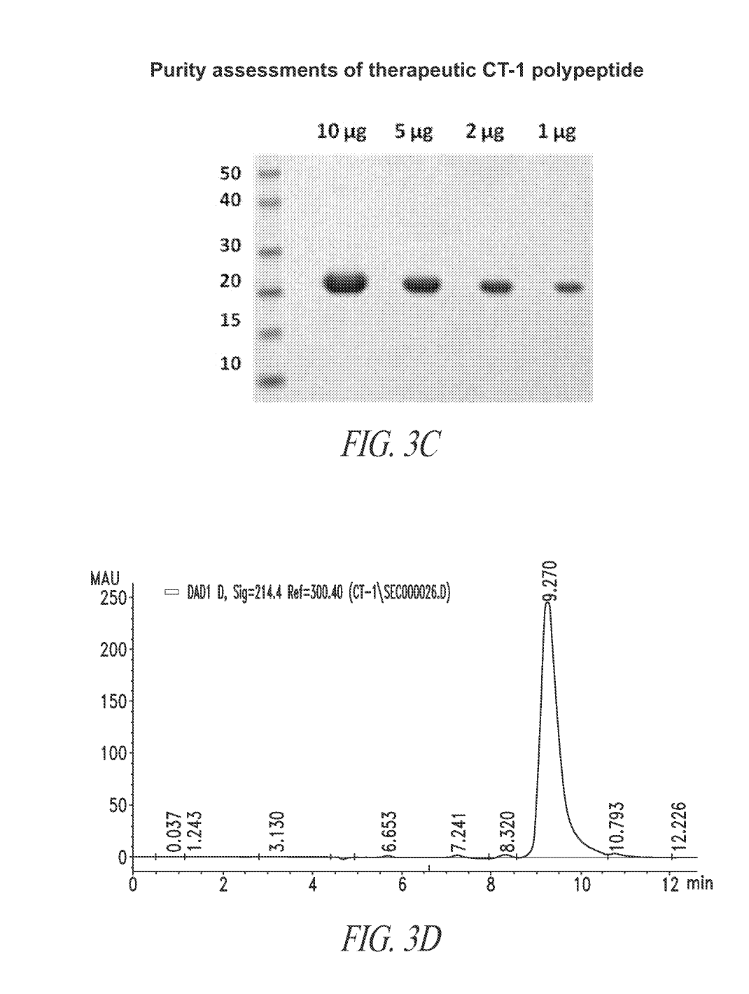

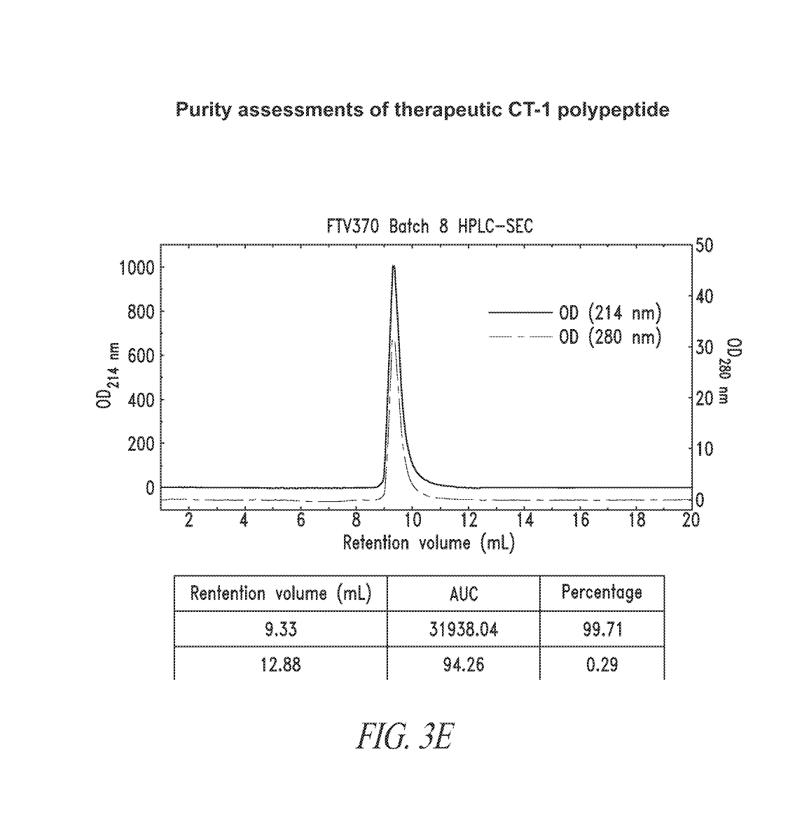

Analysis of the wild-type CT-1 protein suggested there were no mammalian-specific post translational modifications in human CT-1 polypeptide. Therefore, naturally occurring CT-1 and all of the initial CT-1 variants listed in FIG. 2 and SEQ ID NOs: 2-8 were subcloned into expression vectors, transformed into E. coli and expressed as described in Example 1. All polypeptides of CT-1, including wild-type and variants, were expressed to very high levels (400-800 mg/liter shake flask) in bacteria as inclusion bodies. The inclusion bodies were washed, solubilized and refolded as described in Example 1 and outlined in FIG. 3A. Refolded CT-1 proteins were purified away from host cell proteins by ion exchange chromatography and the pure proteins were buffer exchanged into neutral buffer and filtered. All polypeptides, including wild-type human CT-1 and novel variants described herein, were produced in this way. All polypeptides refolded and purified with step-yields between 10-25% of starting material.

### Purity Assessment of Therapeutic CT-1

The CT-1 protein purified and formulated as described in Examples 1 and 3 was then assessed for purity. SDS-PAGE analysis showed the CT-1 protein produced by the processes of the invention to be substantially pure of host cell protein (98.5%) (FIG. 3C). HPLC-SEC analysis demonstrated a pure and homogeneous sample of approximately 20 kDa on comparison with reference standard retention time (FIG. 3D). Samples were further assessed as homogeneous monomeric (99.7%) by peak area analysis (FIG. 3E).

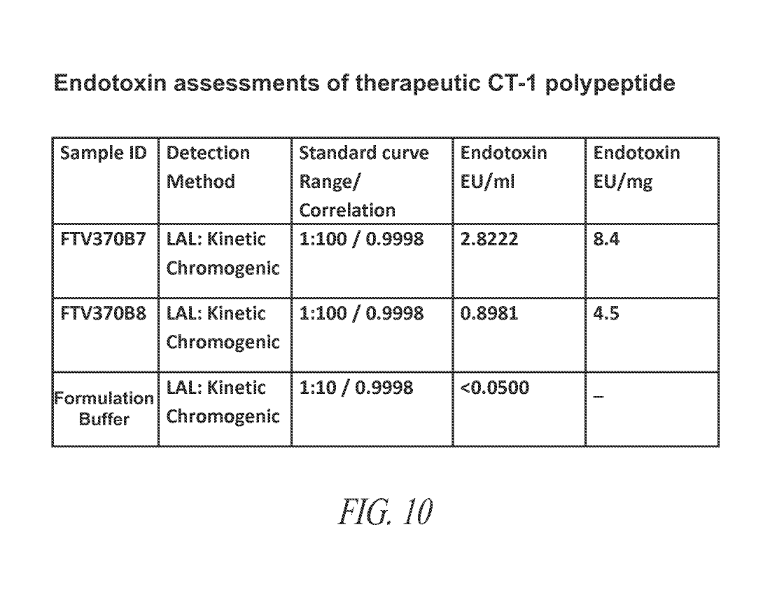

Next, endotoxin levels within batches of the pure proteins were assessed. Endotoxin can be a major problematic contaminant of therapeutic proteins, especially those produced in bacterial systems. High contaminating endotoxin can induce an inflammatory response on therapeutic administration resulting in serious medical complications. The standard assay for endotoxin in aqueous samples and that preferred by regulatory agencies is the Limulus Amebocyte Lysate assay (LAL). Kinetic chromagenic LAL assays were performed on two batches of CT-1 (FTV370). The data can be seen summarized in FIG. 10: the endotoxin levels are very low for the CT-1 protein produced and purified from bacteria using the protocols described herein (range from 4.5-8.4 EU/mg protein; 0.898 EU/ml for wild-type protein).

Proteins were stable at 4° C. and after freeze thaw: with no aggregation, digestion or multimerization as assessed by HPLC-SEC. Biological activities of the novel CT-1 variants in comparison to wild-type were assessed as described in Example 7.

### Example 4

### Assessment of Composition of Therapeutic CT-1

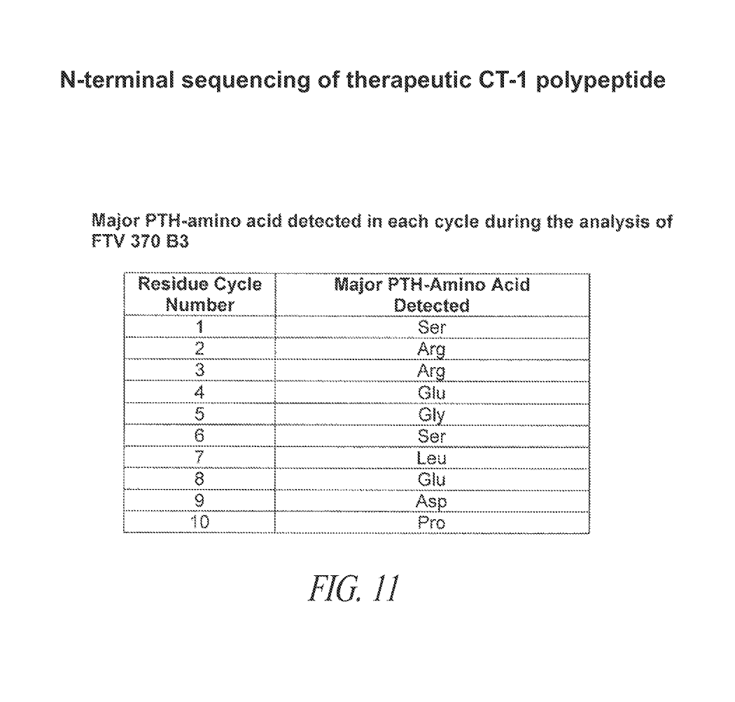

The final composition of wild-type CT-1 produced and purified according to the production methods of the invention, as described above, was then analyzed. Amino-terminal sequencing by Edman degradation was performed: pulsed liquid-phase N-terminal sequencing was carried out using an Applied Biosystems (ABI) 492 automatic protein sequencer. Ten cycles of Edman degradation were carried out with the standard ABI program. The results were reported as the major phenylthiohydantoin (PTH)-amino acid detected for each cycle. The raw chromatogram data is summarized to give the N-terminal sequence listed in FIG. 11.

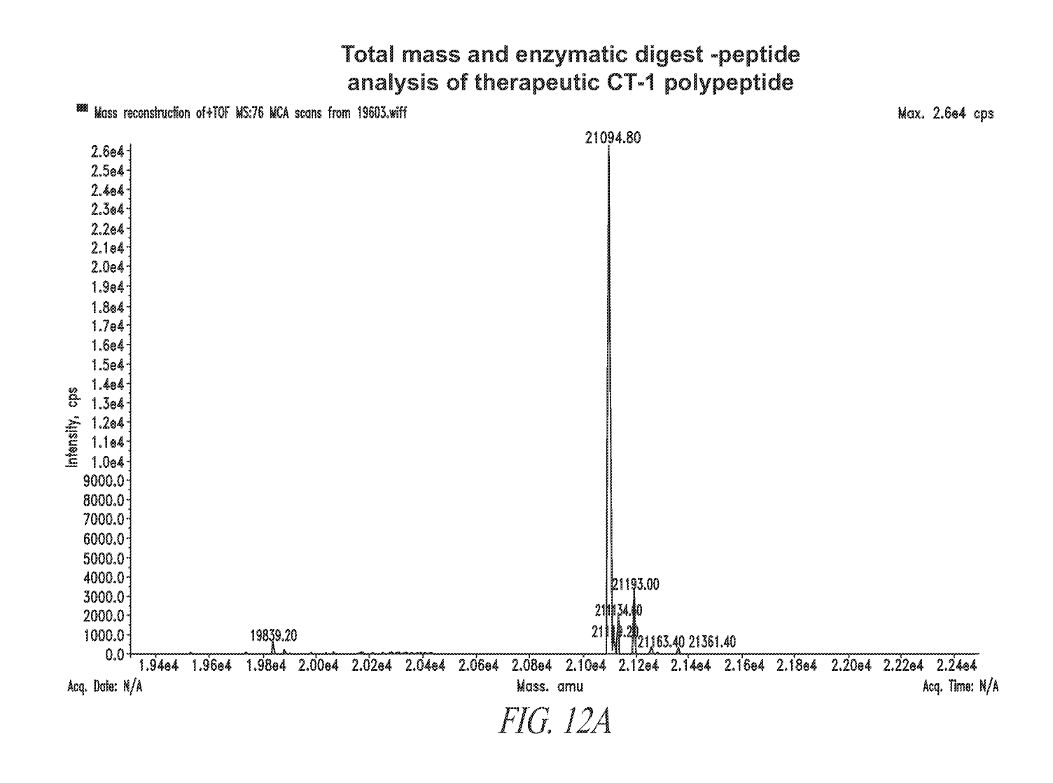

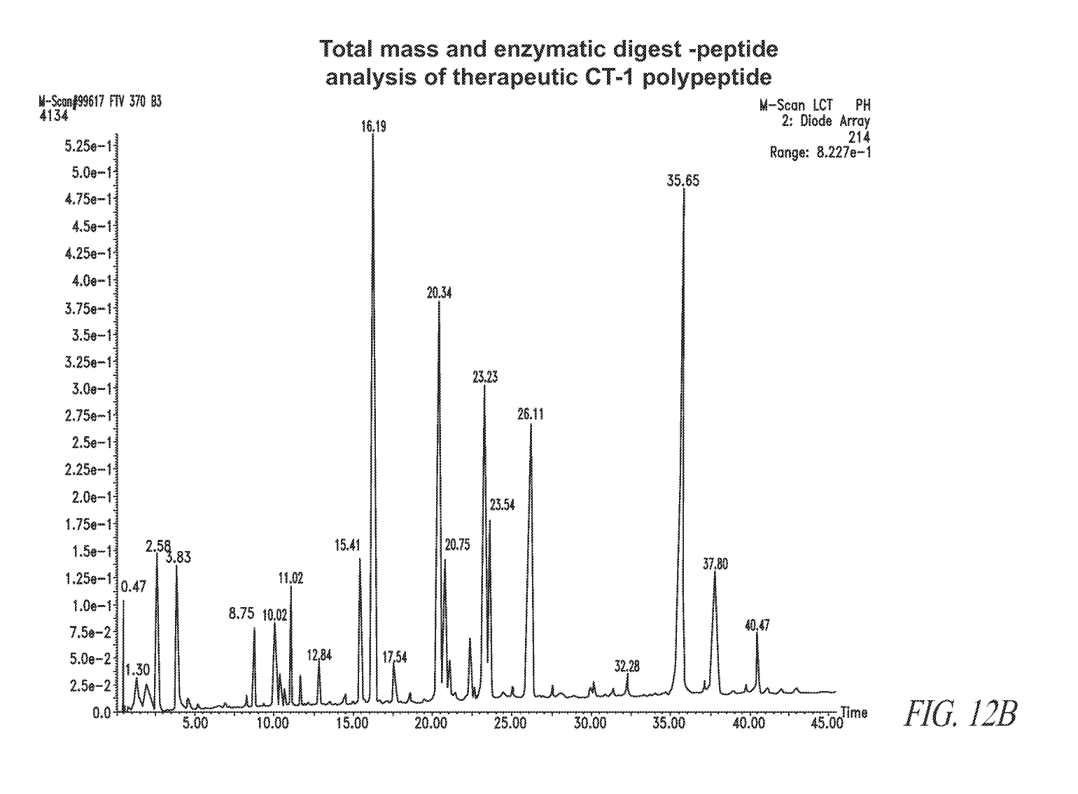

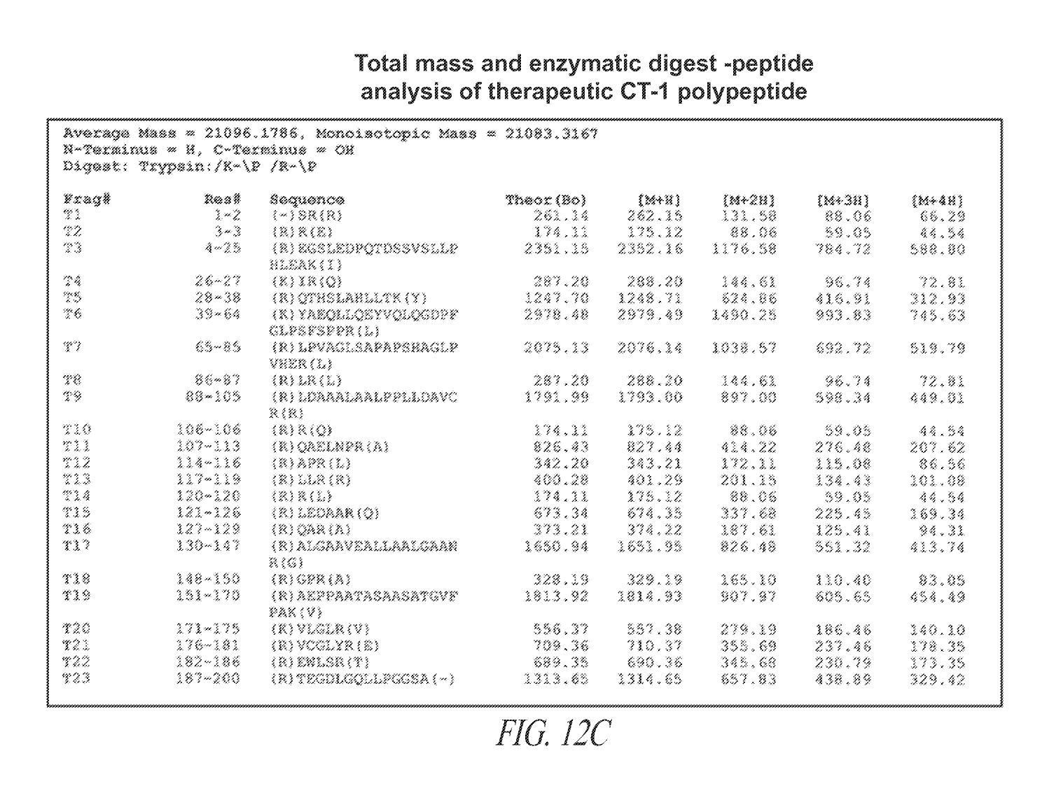

Our results indicate that the first amino acid in the purified form of CT-1 is serine indicating that the N-terminal methionine is cleaved off. This is not always the expectation of recombinant proteins produced in prokaryotic systems. Electrospray ionization spectrometry was used to define the total intact mass of the purified CT-1 protein (Sciex Q-Star/Pulsar). After desalting with a C4 Zip tip and elution with 50% acetonitrile containing 0.1% formic acid, sample aliquots of 1 μl were introduced into the instrument source via nanospray. As can be seen in FIG. 12A, the sample gave a strong positive ion electrospray-MS spectrum with major series of possible multiply charged ions. When deconvoluted the major component was defined with mass of 21094.8 KDa—ostensibly equivalent to the predicted mass of the polypeptide without the N-terminal methionine. Further, Trypsin digest of the pure protein and peptide mapping was completed using ESI-MS spectra of the major UV214 peaks seen in FIG. 12B. Predicted sequences for the peptides are seen tabulated in FIG. 12C and represent a 96.5% coverage of the protein. Collectively, our data show the pure CT-1 protein produced using the methods described herein has the N-terminal methionine removed and does not contain any further post-translational modifications.

### Example 5

### Enhanced Stability of CT-1 Polypeptides Produced by the Methods of the Invention

Therapeutic protein production requires methods of production and formulation to ensure stability of the final product. Aggregation of recombinant proteins at high concentrations can occur if the protein is heterogeneous, inadequately buffered or formulated incorrectly. For therapeutic proteins, aggregation not only affects shelf-life of the product but can also induce antigenic reactions when administered and therefore is a safety risk. Stability assays in which protein integrity is assessed over time at various temperatures such as 4° C.-8° C. or 22° C.-28° C. or 37° C. or on repeated freeze-thaw are standard within the industry. Proteins can be formulated with carrier proteins which aid in soluble aqueous formulation of the protein of interest without effecting biological activity. However, carrier proteins are not a preferred formulation method for therapeutic proteins.

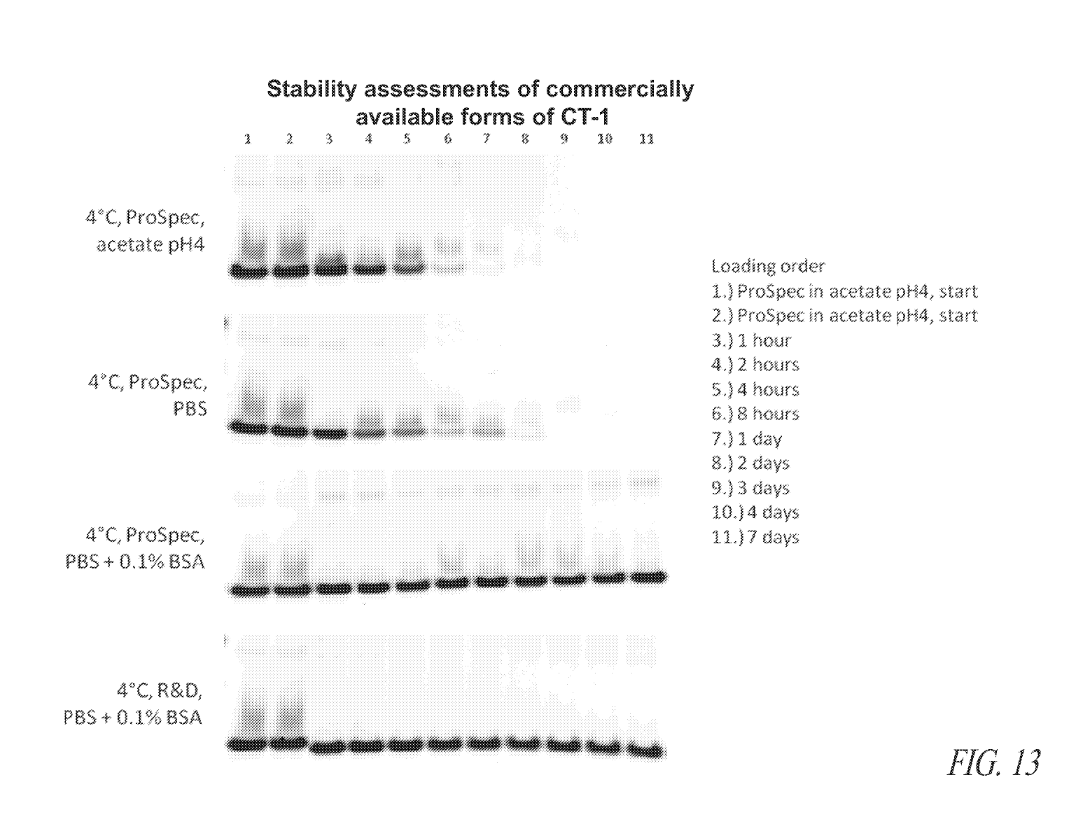

Stability of CT-1 protein from several sources was assessed by sensitive western blot analysis. As can be seen from FIG. 13, commercially available wild-type CT-1 protein from Prospec has a suggested formulation of acetate buffer pH 4.0. With this formulation, the protein was incubated at 37° C. and equal volume samples were drawn, cleared of aggregation by centrifugation and assessed by western blot. Results demonstrate that the protein is ostensibly gone from solution after 8 hours. When the same protein preparation is formulated in a more therapeutically relevant buffer such as phosphate buffered saline (PBS) pH7.4, the degradation of the protein is accelerated and soluble protein levels are dramatically reduced by 2 hours at 37° C.

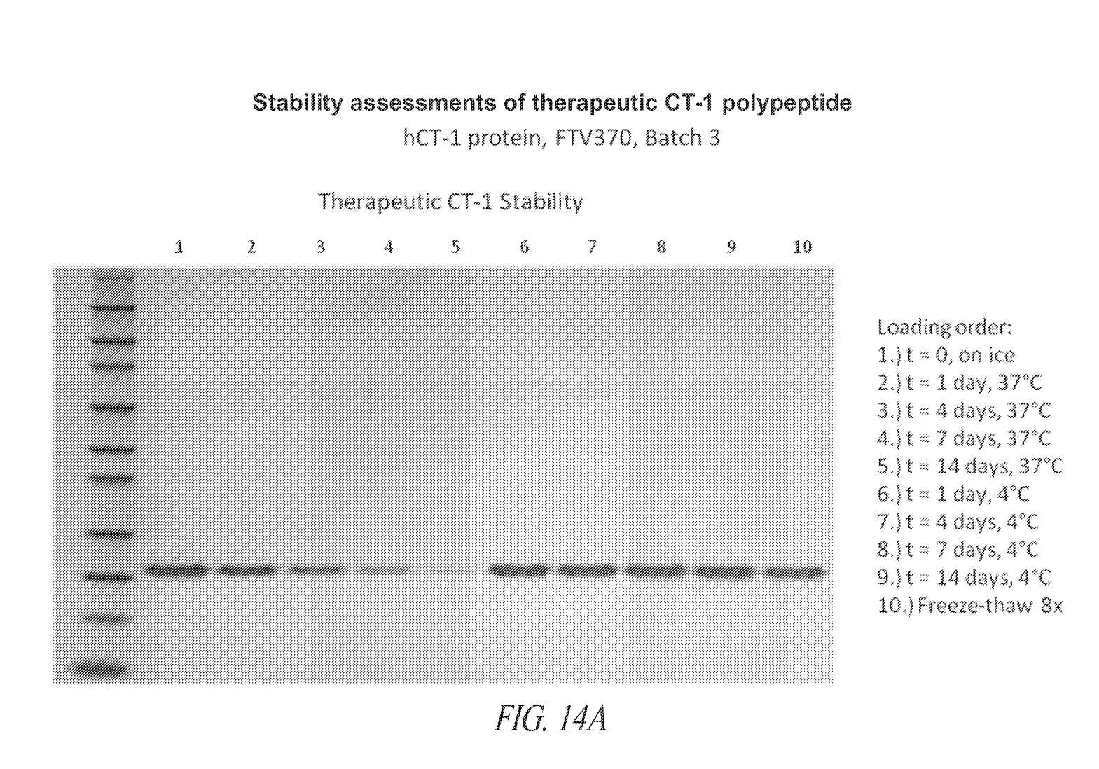

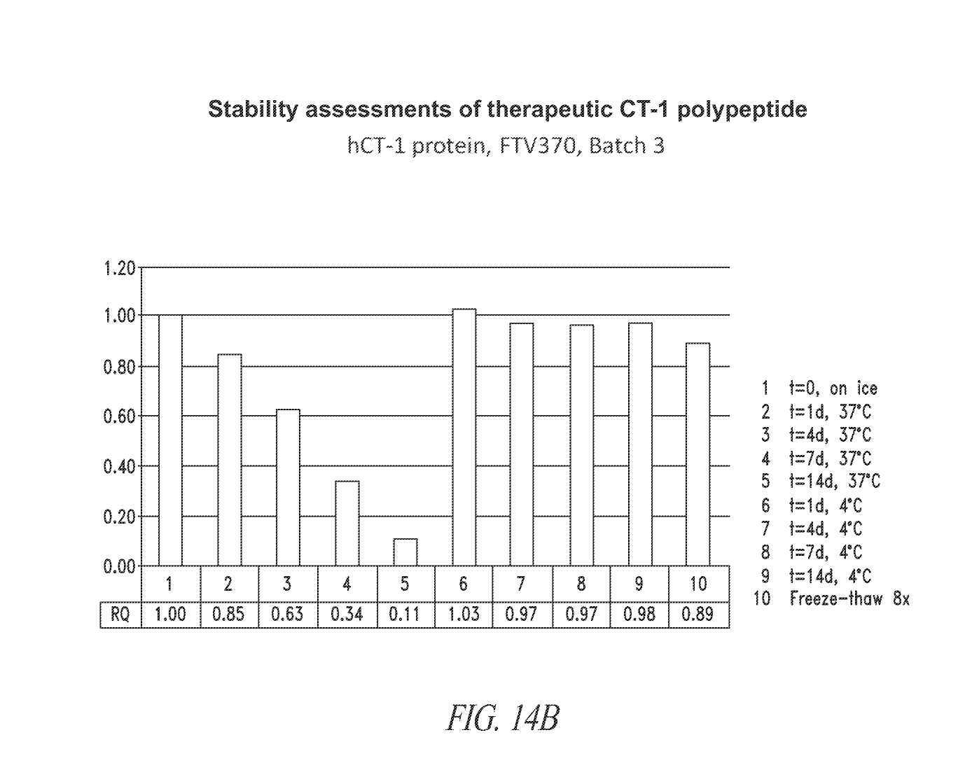

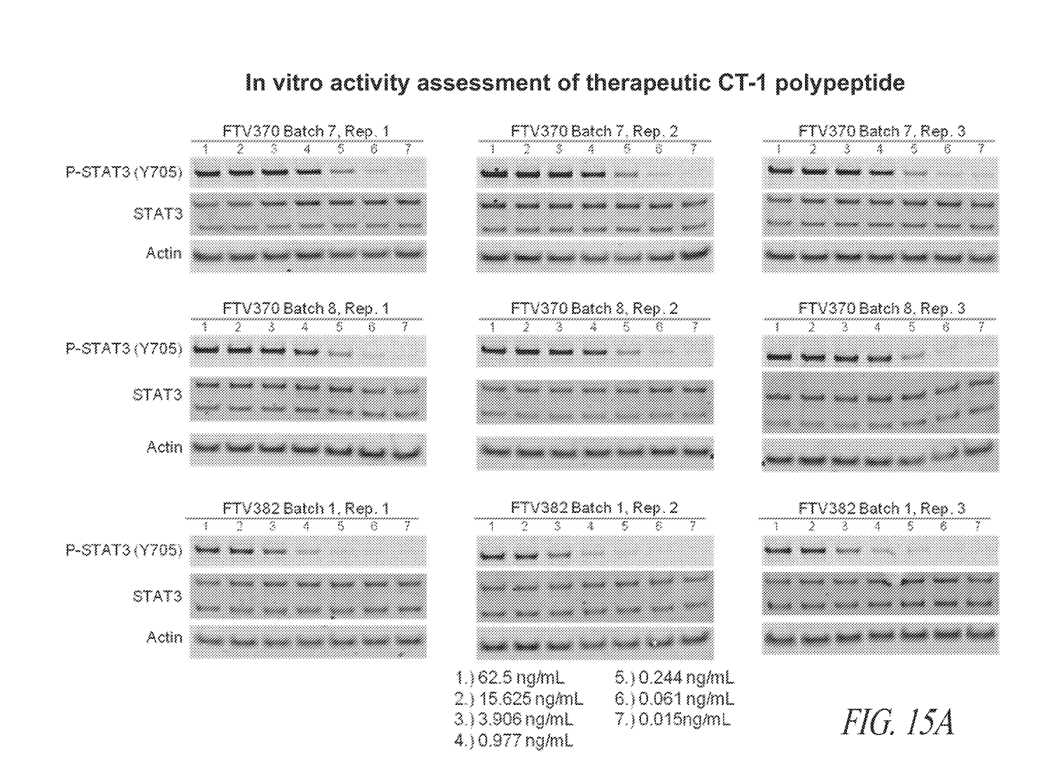

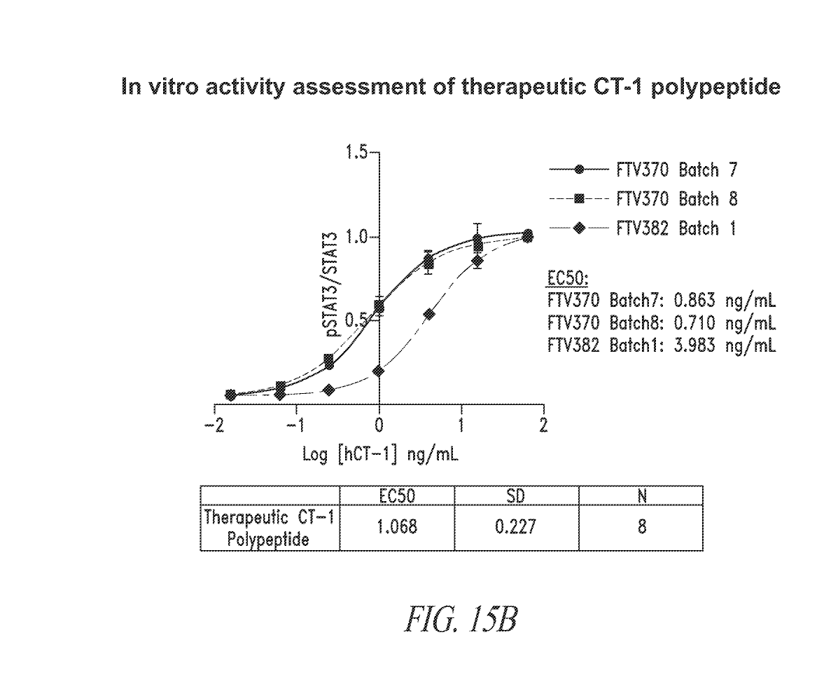

The results of formulating with a carrier protein are shown in FIG. 13 where bovine serum albumin (BSA) enhances stability at 37° C. Protein produced and formulated via the methods of this invention is significantly more stable than the commercial forms without the use of carrier protein. As can be seen in the coomassie-stained SDS-PAGE analysis of FIG. 14A and densitometry quantification of the same in FIG. 14B, while protein is lost from solution over time at 37° C., degradation occurs at a much slower rate in proteins produced and formulated according to the methods of the invention than for the commercial forms. The majority of the protein produced in the methods of the invention remains in solution for over 4 days at 37° C. and detectable levels are seen at 14 days. Further, our results demonstrate that the CT-1 protein produced in the methods of the invention is essentially stable for up to 14 days at 4° C., and remains stable and in solution with little or no aggregation after repeated freeze-thaw cycles.

### Example 6

### Site-Specific Pegylation of Novel CT-1 Variants

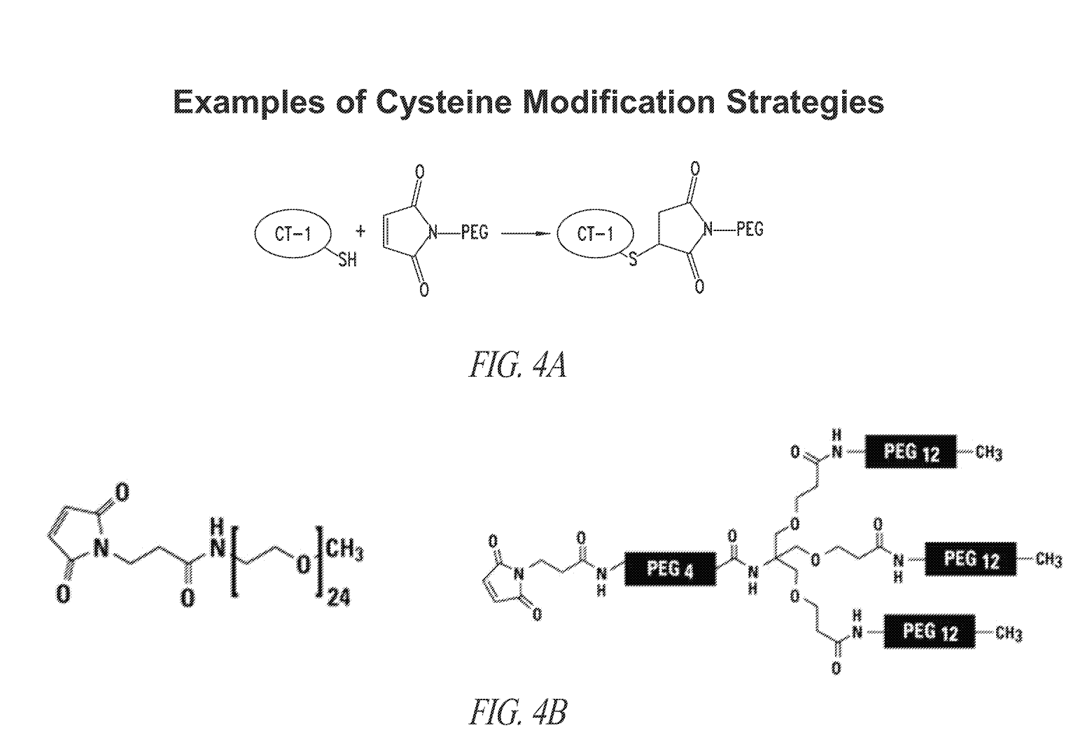

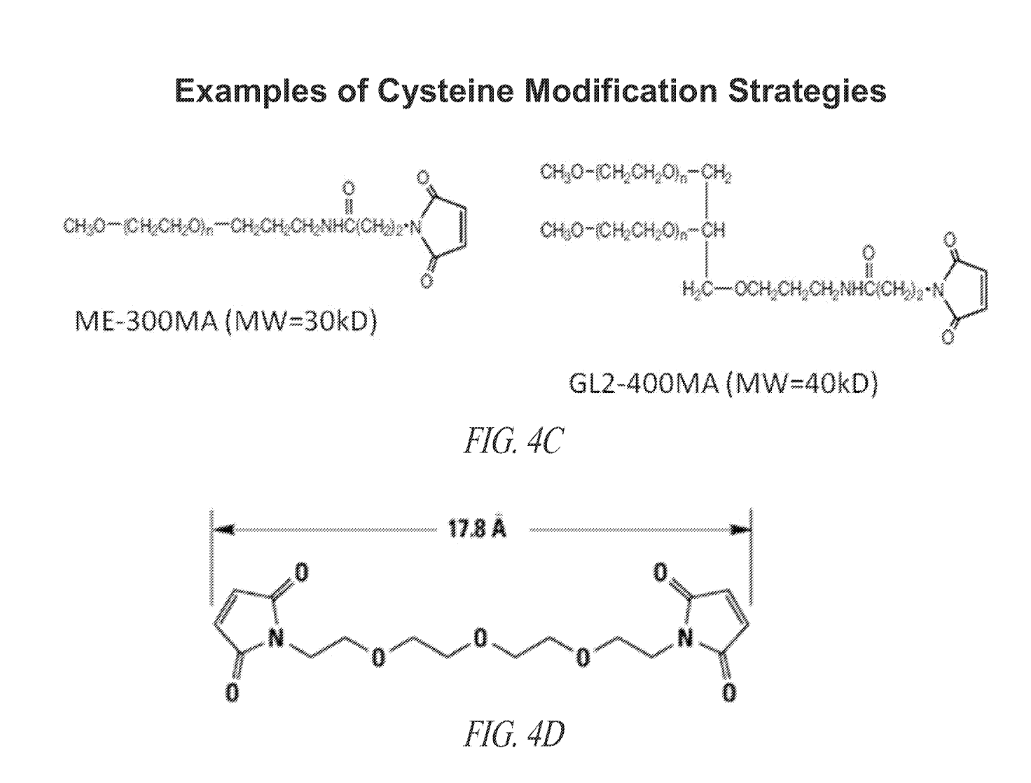

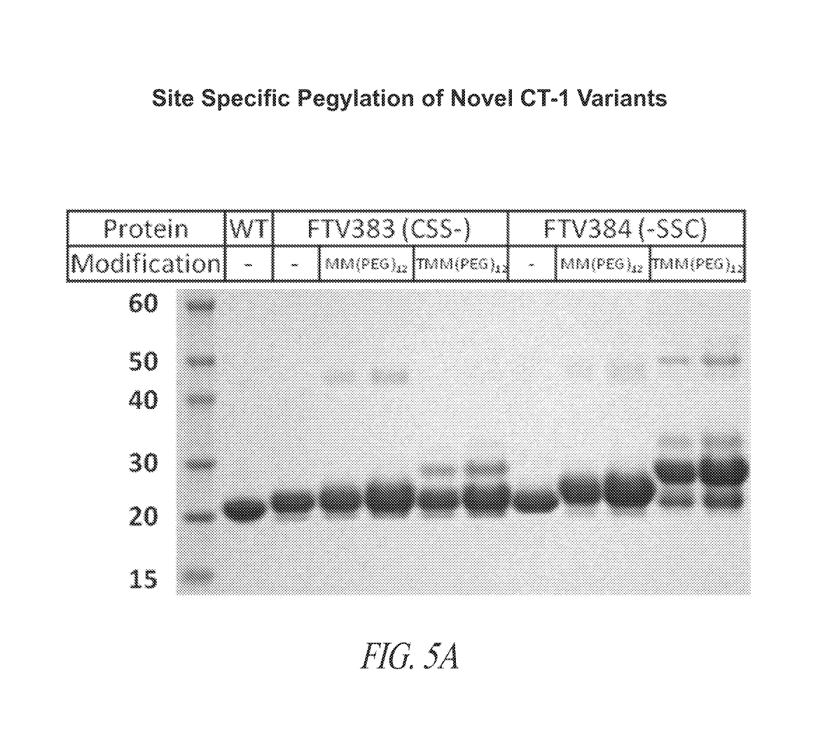

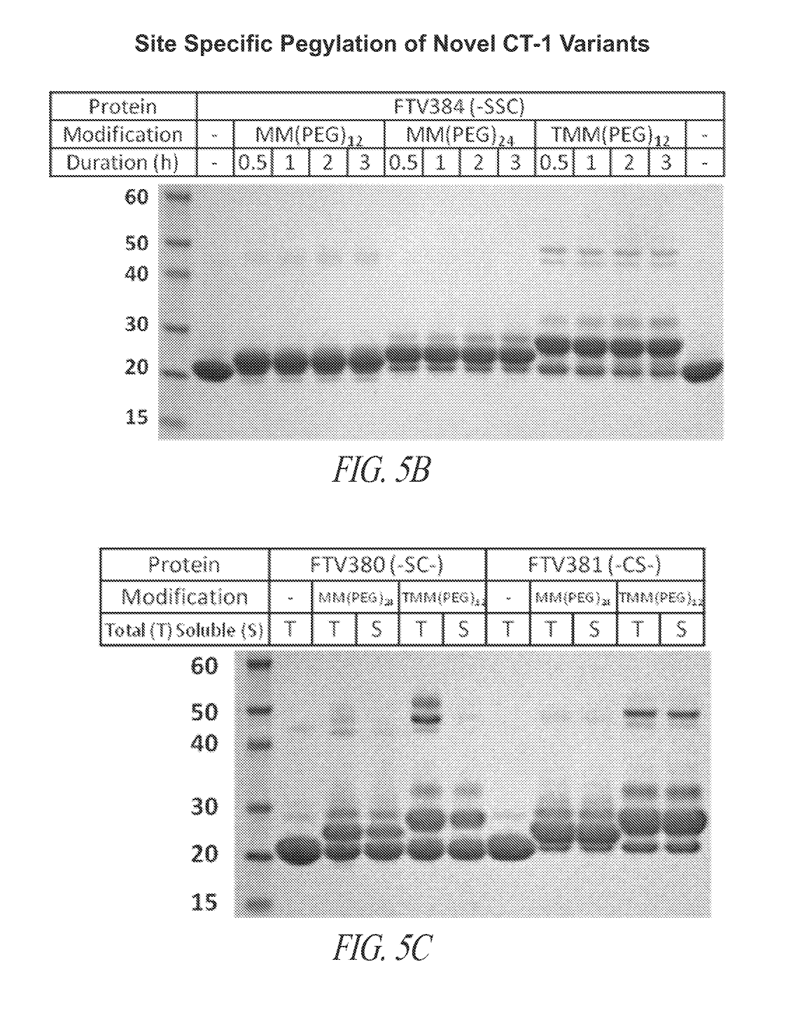

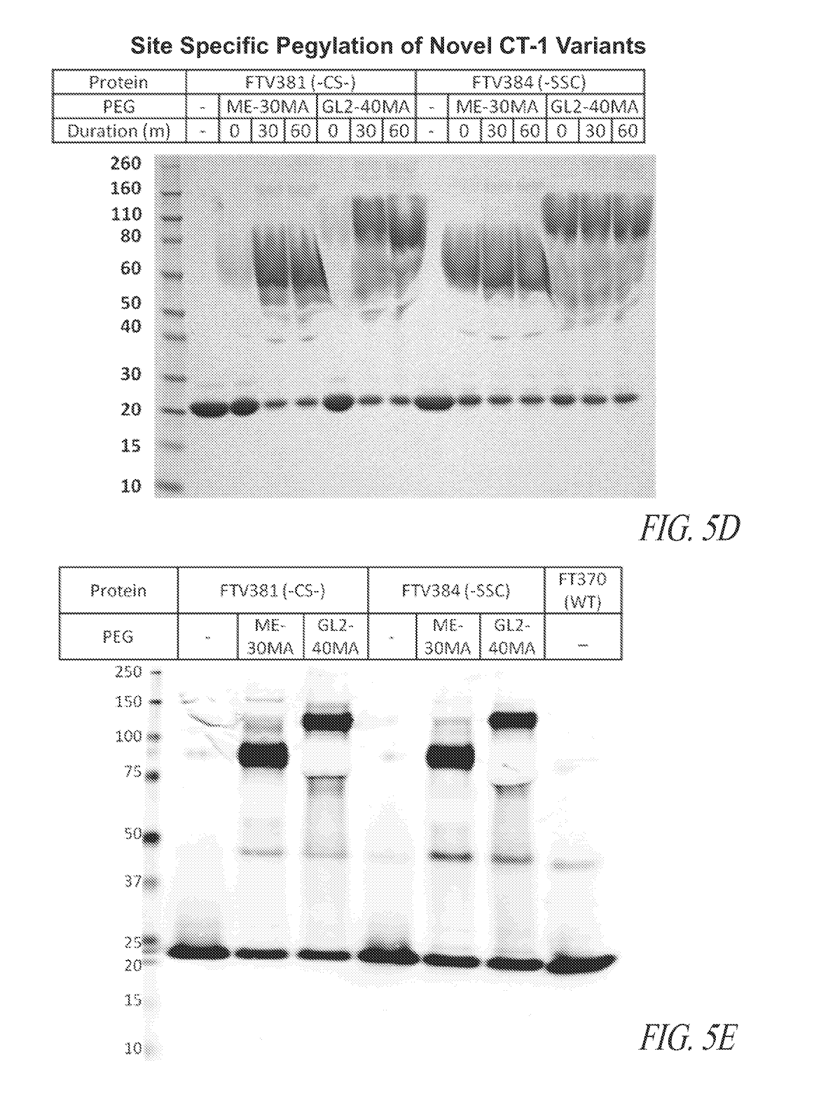

All CT-1 protein variants produced using the methods described in Example 1 and Example 3 were stable and soluble. CT-1 variants with a single cysteine residue were PEGylated in a site specific manner using PEG molecules of various lengths and with either single or branched chains linked to maleimide active groups. At neutral pH the maleimide only reacted with the single thiol side chain provided by the cysteine residues. Structural examples of the types of PEG molecule used are depicted in FIGS. 4B and 4C. Coomassie-stained SDS-PAGE depicting the molecular weight changes associated with site specific modification of the CT-1 novel variant proteins with PEG molecules are seen in FIG. 5. MM(PEG)₁₂ denotes a linear chain PEG with 12 units, MM(PEG)₂₄ denotes a linear chain PEG with 24 units and TMM(PEG)₁₂ denotes a branched chain PEG with 12 units/chain. Protein FTV383 which has both naturally occurring cysteine residues replaced with serines and an additional cysteine residue at its amino terminus was PEGylated with single chain or branched chain molecules via thiol-maleimide conjugation albeit with low efficiency (FIG. 5A). In contrast FTV384 which has both naturally occurring cysteine residues replaced with serines and an additional cysteine residue at its carboxyl terminus was PEGylated with single chain or branched chain molecules with high efficiency with almost no residual unmodified protein after a 30 minute reaction (FIGS. 5A and B). FTV380 which has the naturally occurring cysteine at position 105 replaced with serine was PEGylated at the remaining cysteine (178) albeit with low efficiency and resulted in an unstable product (FIG. 5C). In contrast, FTV381 which retains the cysteine at position 105 and has cysteine 178 replaced was PEGylated with high efficiency and resulted in a stable product (FIG. 5C). Therefore certain sites were more permissible to PEGylation than others with respect to access and/or stability of modified product.

In a further demonstration, the two CT-1 variants exhibiting the most efficient and stable PEGylation with small molecular weight modifications (FTV381 and FTV384) were conjugated with much larger PEG molecules. 30 KDa single branch or 40 KDa multi branched PEG molecules were conjugated to CT-1 variants (FIG. 4C). As with the smaller PEG molecules site specific modification was via the maleimide-thiol conjugation and the reaction proceeded with 10× molar excess of PEG over protein. As can be seen in the coomassie stained SDS-PAGE in FIG. 5D both FTV381 and FTV384 was efficiently PEGylated with large molecular weight PEG molecules and resulted in stable products. On a further assessment of the nature of the PEGylation, an anti-CT-1 western blot was performed to ensure a single PEGylated product resulted from the reaction. The resulting blot in FIG. 5E clearly shows a single large molecular weight modified CT-1 variant product.

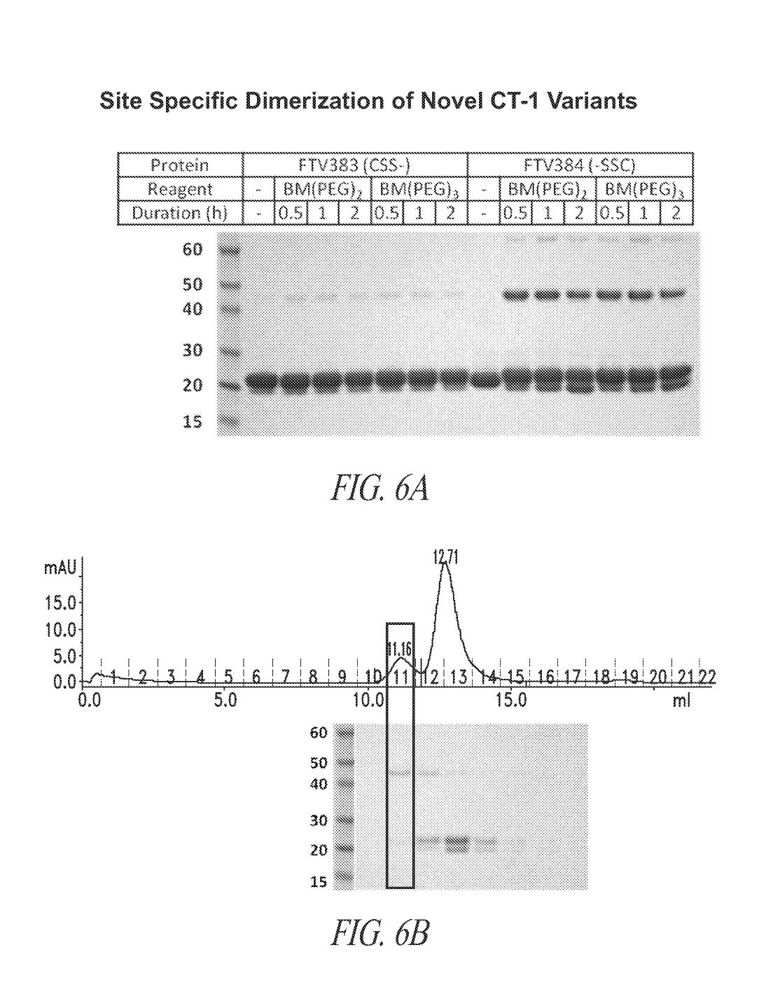

In a further demonstration of the modification potential of the novel CT-1 compositions, proteins were homodimerized by chemical conjugation. The dimerization reaction utilized a flexible PEG linker with two maleimide groups such as that depicted in FIG. 5C. As can be seen in FIG. 6, while the construct with a single amino terminal cysteine (FTV383) was dimerized to a degree the process was inefficient, with a low yield of dimer product. In contrast the same reaction using a CT-1 variant with a single carboxy-terminal cysteine was efficiently dimerized using either a 2 or 3-unit PEG linker.

Thus, the novel CT-1 compositions that were engineered to comprise only one cysteine and therefore a single site of potential modification with a sulfydryl-reactive species were able to be modified to form stable products. Some sites of modification were more permissible than others: the cysteine residue at position 105 in CT-1 was effectively modified whereas conjugation of PEG to the cysteine at position 178, while feasible, resulted in a somewhat unstable product. The constructs generated to include single cysteine residues at the amino or carboxy termini demonstrated that the carboxy terminus was more permissible to modification that the amino terminus. Dimerization of novel CT-1 variants by chemical conjugation was demonstrated.

### Example 7

### Activity Assessments of Novel CT-1 Variants and Identification of Variants with Improved Activities Over Wild-Type

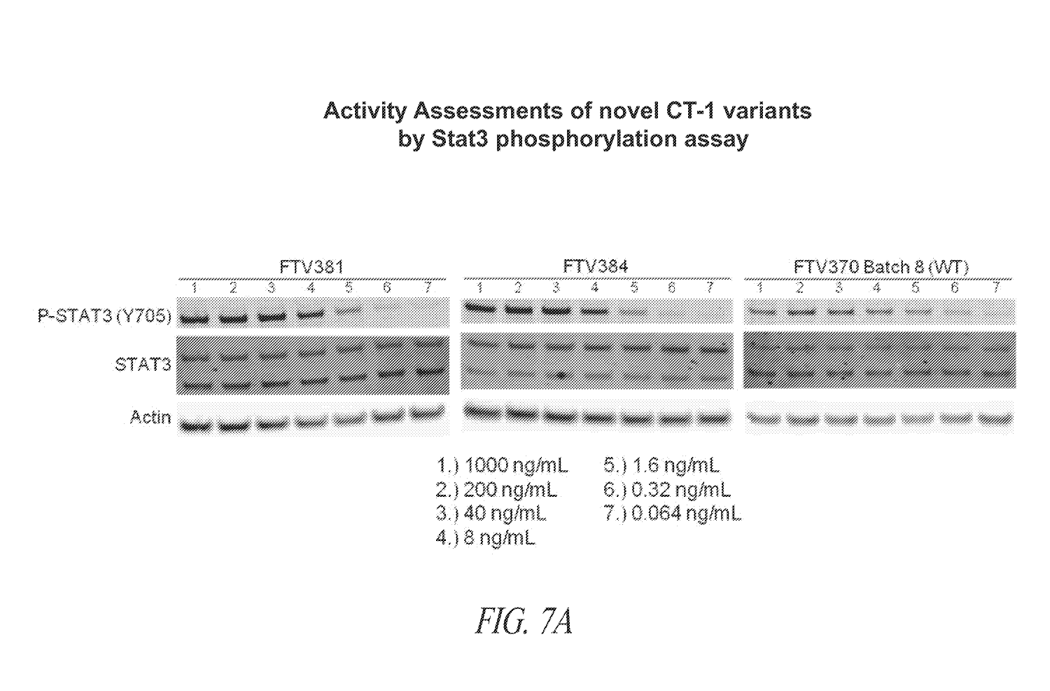

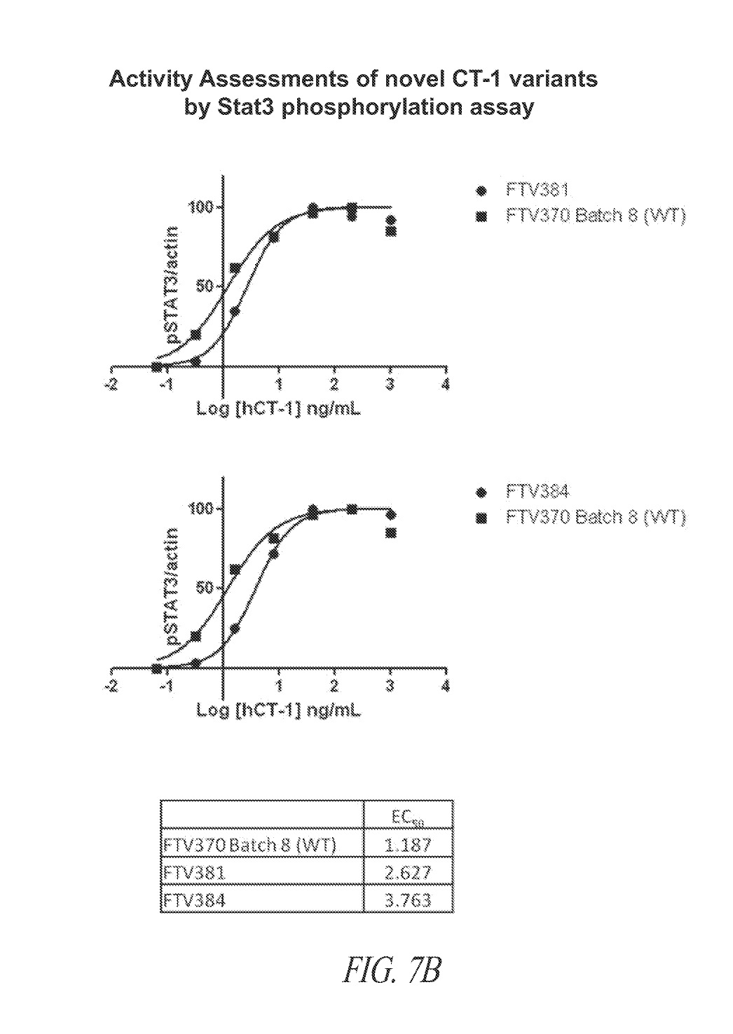

CT-1 polypeptides expressed and purified via the methods described in Examples 1 and 3, including the novel CT-1 variant proteins, were next assessed for CT-1 biological activity. CT-1 is a potent activator of gp130-mediated signaling pathways such as JAK/Stat and ERK. We developed a sensitive assay for the measurement of Stat3 phosphorylation using the method described in Example 1. FIG. 7 shows a typical activity assay in which wild-type CT-1 activity is compared to two of the novel variants. Stat3 phosphorylation on treatment of C212 cells with titrations of the CT-1 proteins was assessed by western blot. Densitiometry quantification normalized to total Stat and/or actin was quantified as titration curves and mean EC50 values compared between variants. For example, and as described in FIG. 7, wild-type CT-1 has an EC50 value of approximately 1 ng/ml and the variants FTV381 and FTV384 have EC50 s between 2-5 and 4 ng/ml.

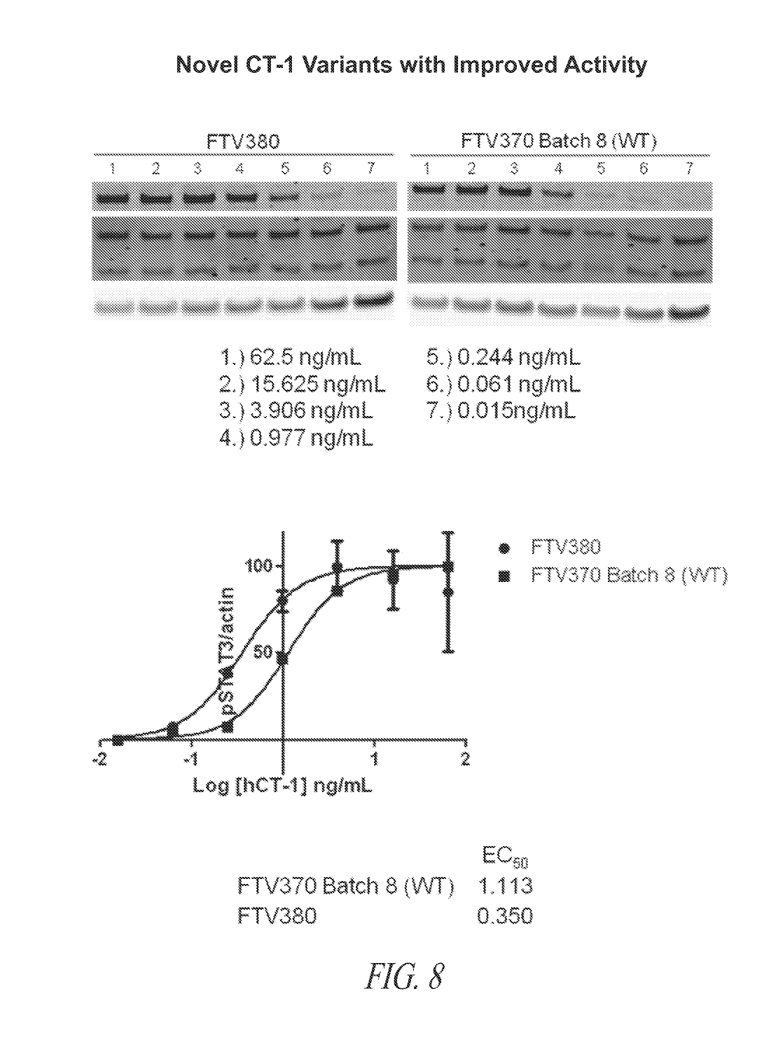

In an unexpected finding, a novel CT-1 variant with the naturally occurring cysteine residue at the position corresponding to position 105 in SEQ ID No: 2 exchanged for serine (FTV380) demonstrated reproducibly higher biological activity than the wild type protein. Exemplary data for this finding is shown in FIG. 8 where FTV380 has a demonstrated EC50 of 0.35 ng/ml compared to the wild-types EC50 of approximately 1 ng/ml. Therefore other amino acid changes at this position or within this area of the protein were not only permissible for activity but increased biological activity relative to the wild-type CT-1.

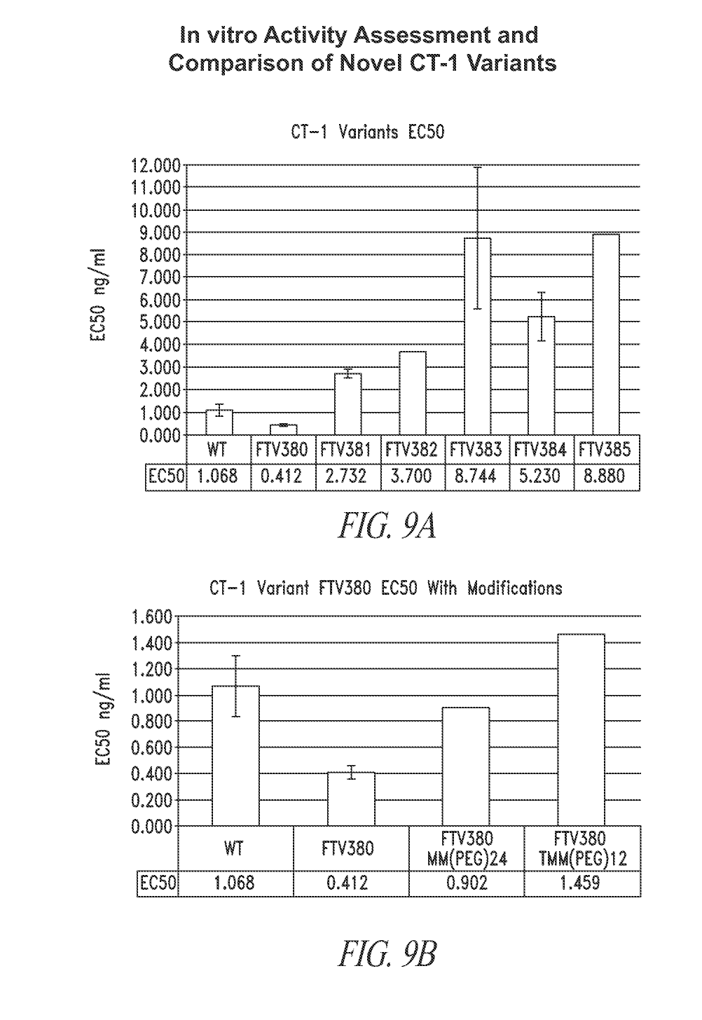

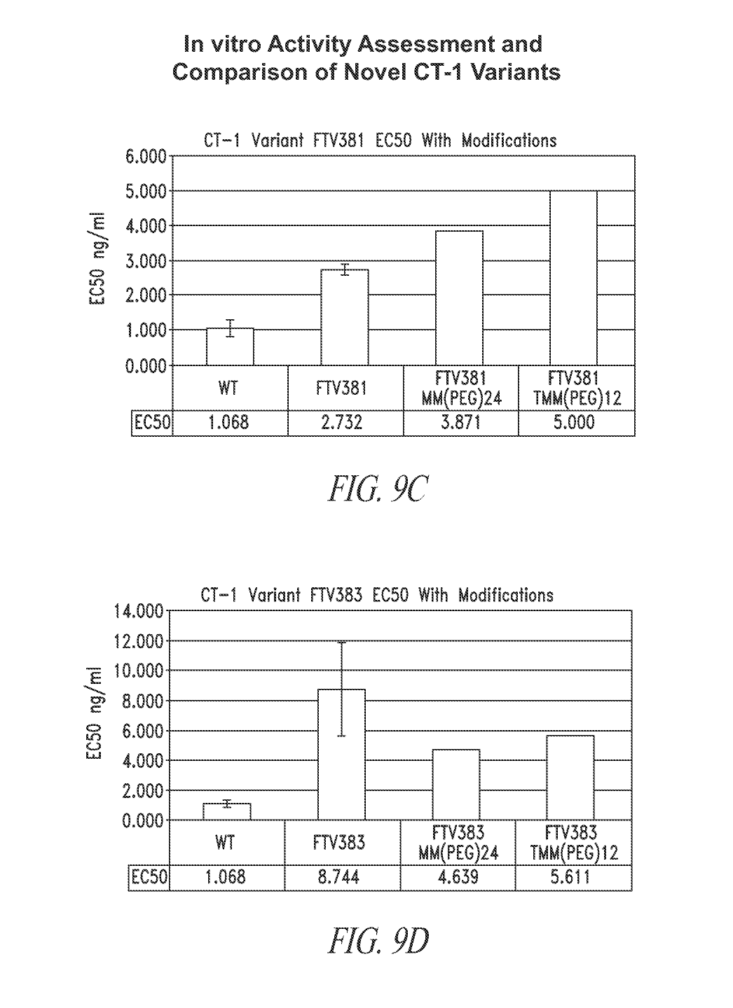

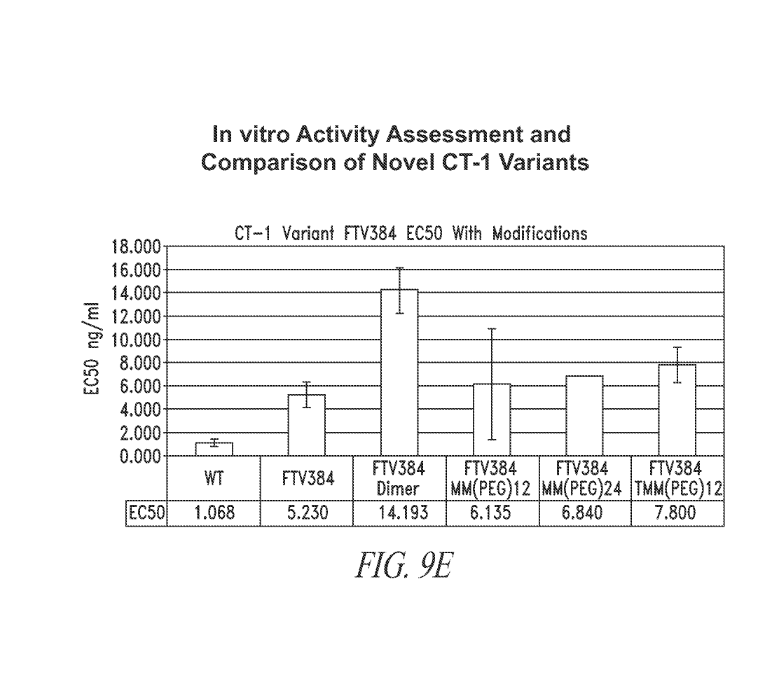

A summary of relative biological activities for the various novel CT-1 variants on comparison with wild-type CT-1 is shown as mean EC50 values in FIG. 9A. While activities varied, all variants retained CT-1 biological activity. Notably FTV381 and FTV384 retained relatively high biologically activities with EC50 values of approximately 2.5 ng/ml and 5 ng/ml respectively.

Relative EC50 values for the various proteins in both modified and unmodified form are shown in FIGS. 9B, 9C, 9D and 9E. As described in Example 6, the relative efficiency of modification and stability of modified product was site-specific with some sites more permissible than others. Biological activity of modified proteins was substantially maintained suggesting the modifications were permissible with respect to CT-1 signaling activity. Specifically, novel variants FTV381 and FTV384 which were demonstrated to be efficiently modified to obtain stable PEGylated protein substantially retained their biological activity post modification.

Post-modification CT-1 signaling activity of the novel CT-1 variant FTV384 was retained as a purified dimer (FIG. 9E).

The assessment of modification efficiency and biological activity suggests that novel compositions with site specific modifications had CT-1 biological activity.

### Example 8

### Pharmacokinetics Analysis of Novel CT-1 Variants

The pharmacokinetic analysis of wild-type CT-1 shows that it has a very short systemic half-life irrespective of administration route: intravenous, subcutaneous and intramuscular administrations results in dose-dependent systemic levels of protein with half-life in the 2-4 hour range. The modification of CT-1 to increase its molecular weight from 21.5 KDa to 45, 50, 60, 70, 80 . . . KDa will substantially increase systemic half-life by decreasing clearance and in the case of compartmentalized administration by decreasing absorption into systemic circulation. The novel CT-1 variants described within this application comprising site-specific modifications to increase molecular weight while retaining biological activity may have greater therapeutic utility due to improved bioavailability and administration interval.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A composition comprising an isolated protein of at least 160 amino acids identical to the amino acid sequence set forth in SEQ ID NO: 2 and a protein modification of SEQ ID NO: 2 that includes one or more of the following:
a) a Cys or a Lys at a non-naturally occurring amino acid position in SEQ ID NO: 2;
b) at least one deletion of Cys 105 or Cys 178;
c) at least one substitution of Cys 105 or Cys 178 with another amino acid; and
d) addition of a pharmokinetic (PK) modulator.

2. The composition of claim 1, wherein the isolated protein comprises at least 160 contiguous amino acids identical to the amino acid sequence set forth in SEQ ID NO: 2.

3. The composition of claim 1, wherein the isolated protein comprises
a) at least 160 contiguous amino acids identical to the amino acid sequence set forth in SEQ ID NO: 2; and
b) at least one disruption of 1 to 7 amino acids in the contiguous amino acid sequence set forth in SEQ ID NO: 2.

4. The composition of claim 1, wherein the Cys at the non-naturally occurring amino acid position in SEQ ID NO: 2 is within about 10 amino acids of the N- or C-terminus of the isolated protein.

5. The composition of claim 1, wherein the PK modulator is a polyethylene glycol (PEG) and Cys 105 and Cys 178 are substituted with glycine (G), serine (S), alanine (A), threonine (T), leucine (L), isoleucine (I), or arginine (R).

6. The composition of claim 1, wherein said PK modulator is a PEG and either Cys 105 or Cys 178 are substituted with G, S, A, T, L, I, or R.

7. The composition of claim 1, wherein the composition comprises:
a) a PK modulator comprising a PEG of at least 10 kD;
b) about 1 PEG moiety per about 1 molecule of isolated protein; and
c) the isolated protein is at least 95% pure by weight.

8. The composition of claim 1, wherein the composition comprises:
a) a PK modulator comprising a PEG of at least 10 kD;
b) about 2 PEG moieties per about 1 molecule of isolated protein; and
c) the isolated protein is at least 95% pure by weight.

9. The composition of claim 1, wherein the composition comprises:
a) a PK modulator comprising a PEG of at least 10 kD;
b) about 0.5 PEG moieties per about 1 molecule of isolated protein; and
c) the isolated protein is at least 95% pure by weight.

10. The composition of claim 3, wherein the PK modulator is a PEG and either Cys 105 or Cys 178 are substituted with G, S, A, T, L, I, or R, and the isolated protein has activity in a cell-based or biochemical assay.

11.-21. (canceled)

22. An isolated biologically active polypeptide comprising an amino acid sequence as set forth in SEQ ID NO: 23, wherein
Xaa at position 1 is absent or methionine;
Xaa at position 2 is absent or any amino acid;
Xaa at position 203 is absent or any amino acid; and
wherein the polypeptide comprises one or more amino acid substitutions, deletions, or modifications at one or both of amino acid positions 106 or 179.

23. The polypeptide of claim 22, wherein
Xaa at position 2 is selected from the group consisting of: cysteine, lysine, histidine, asparagine, aspartate, glutamate, alanine, glycine, threonine, serine, and valine.

24. The polypeptide of claim 22, wherein
Xaa at position 203 is cysteine.

25. The polypeptide of claim 22, wherein the polypeptide comprises a chemical modification.

26. The polypeptide of claim 25, wherein the chemical modification comprises pegylation.

27.-40. (canceled)

41. A polynucleotide encoding the polypeptide of claim 22.

42. A vector comprising the polynucleotide of claim 41.

43.-44. (canceled)

45. A host cell comprising the vector of claim 42.

46. (canceled)

47. A polypeptide produced by the host cell of claim 45.

48. A composition comprising the polypeptide of claim 22.

49.-101. (canceled)
